(19) United States
(12) Patent Application Publication
(10) Pub. No.: US 2023/0039816 A1
(43) Pub. Date: Feb. 9, 2023

(54) METHOD FOR INDUCING HEPATOCYTE PLASTICITY

(71) Applicant: Public University Corporation Yokohama City University, Kanagawa (JP)

(72) Inventors: Hideki Taniguchi, Yokohama-shi, Kanagawa (JP); Yunzhong Nie, Yokohama-shi, Kanagawa (JP); YunWen Zheng, Yokohama-shi, Kanagawa (JP)

(73) Assignee: Public University Corporation Yokohama City University, Kanagawa (JP)

(21) Appl. No.: 17/762,908

(22) PCT Filed: Sep. 24, 2020
(86) PCT No.: PCT/JP2020/036043

§ 371 Date: Oct. 7, 2022

(30) Foreign Application Priority Data
Sep. 27, 2019 (JP) 2019-177843

(51) Int. Cl. C12N 5/071

(52) U.S. Cl. CPC C12N 5/067; C12N 5/0672; C12N 5/0679; C12N 2501/115; C12N 2501/12; C12N 2501/2306; C12N 2501/999; C12N 2506/45

(57) ABSTRACT

The disclosure provides a method for producing hepatocytes or hepatic progenitor cells; a method for suppressing hepatocyte aging using a drug causing histone hyperacetylation; a hepatocyte anti-aging agent; a method for increasing hepatocyte plasticity; and a drug for increasing hepatocyte plasticity.

### TECHNICAL FIELD

The present invention relates to a method for inducing hepatocyte plasticity.

### BACKGROUND ART

Hepatocytes constitute about 70% of liver tissue. Hepatocyte plasticity plays a critical role in maintaining liver regenerative capacity (Nat Cell Biol. 18(3):238-45(2016) (Non Patent Literature 1); Hepatology. 64(6):2244-6(2016) (Non Patent Literature 2)). In other words, it has been found that when liver diseases occur in mice, mature hepatocytes convert into proliferative hepatic progenitor cells to repair the lost hepatocytes and cholangiocytes (Cell Stem Cell. 15(3):340-9(2014) (Non Patent Literature 3); Cell Stem Cell. 15(5):605-18(2014) (Non Patent Literature 4); Cell. 157, 1324-38(2014) (Non Patent Literature 5); Genes Dev. 27(7):719-24(2013) (Non Patent Literature 6)). Such hepatic progenitor cells are also detected in a cirrhotic human liver (Cell Stem Cell. 23(1):114-22(2018) (Non Patent Literature 7)). Moreover, liver regenerative capacity is associated with hepatocyte aging, and liver grafts from elderly donors have been shown to have significantly poorer survival rates (J Hepatol. 70(4):745-58(2019) (Non Patent Literature 8); J Hepatol. 57(2):288-96(2012) (Non Patent Literature 9)).

T. Ochiya et al. show that getting small molecule compounds (YAC, Y-27632, A83-01, CHIR99021) to act on mouse and rat hepatocytes in vitro can induce hepatic progenitor cells having a proliferative ability from mature hepatocytes and allow the cells to be passaged 20 times or more (Cell Stem Cell. 20(1):41-55(2017) (Non Patent Literature 10)). Many research groups subsequently have attempted to apply this culture technique to human hepatocytes. However, the differences in the properties of human hepatocytes and rodent hepatocytes are so great that the induced human hepatic progenitor cells cannot be passaged three times or more (Cell Stem Cell. 23(6):806-819, 2018 (Non Patent Literature 11); Cell Research. 29, 8-22, 2019(Non Patent Literature 12); J Hepatol. 70(1):97-107, 2019(Non Patent Literature 13)). In addition, not only the proliferative status but also pluripotency for differentiation into human hepatocytes and cholangiocytes was not maintained, and induction of hepatic progenitor cells based on the plasticity regulation on human hepatocytes has not been realized.

### CITATION LIST

### Non Patent Literature

Non Patent Literature 1: Nat Cell Biol. 18(3): 238-45 (2016)
Non Patent Literature 2: Hepatology. 64(6): 2244-6 (2016)
Non Patent Literature 3: Cell Stem Cell. 15(3): 340-9 (2014)
Non Patent Literature 4: Cell Stem Cell. 15(5): 605-18 (2014)
Non Patent Literature 5: Cell. 157, 1324-38 (2014)
Non Patent Literature 6: Genes Dev. 27(7): 719-24 (2013)
Non Patent Literature 7: Cell Stem Cell. 23(1): 114-22 (2018)
Non Patent Literature 8: J Hepatol. 70(4): 745-58 (2019)
Non Patent Literature 9: J Hepatol. 57(2): 288-96 (2012)
Non Patent Literature 10: Cell Stem Cell. 20(1): 41-55 (2017)
Non Patent Literature 11: Cell Stem Cell. 23(6): 806-819, 2018
Non Patent Literature 12: Cell Research. 29, 8-22, 2019
Non Patent Literature 13: J Hepatol. 70(1): 97-107, 2019

### SUMMARY OF INVENTION

### Technical Problem

The prior art has the following drawbacks:

1. The method for inducing hepatic progenitor cells from rodent hepatocytes cannot be applied to human cells.
2. The hepatic progenitor cells induced by conventional methods have neither high proliferative ability nor pluripotency.
3. There is no technology to regulate the plasticity of human hepatocytes and aging.

It is an object of the present invention to overcome these drawbacks.

### Solution to Problem

The present inventors have found a novel method for inducing iPS cell-derived hepatocytes and have succeeded in producing hepatocytes capable of maintaining hepatic function for a long time with gradual accumulation of aging-related markers and characteristics.

In addition, the present inventors have newly found that in contrast to rodent hepatocytes, human hepatocytes have their plasticity regulated by the FGF2-MAPK-EZH2 axis. That is, it has been found that human hepatocytes can be converted into hepatic progenitor cells having a proliferative ability and pluripotency (bipotential property of being able to differentiate into hepatocytes and cholangiocytes) by increasing the transcriptional activity of EZH2 through addition of FGF2, whereas this plasticity is impaired in old hepatocytes.

In addition, the present inventors have demonstrated that the decline in histone acetylation is a factor that inhibits these old cells from acquiring plasticity. The present inventors have also found that selective inhibition of histone deacetylases (HDAC) can improve the plasticity of old hepatocytes derived from human iPS cells and primary human hepatocytes derived from the elderly (78 years old). Hepatic progenitor cells artificially induced from these old cells can be passaged 20 times or more in an in vitro culture system and can be applied to the mass production of hepatocytes.

The summary of the present invention is as follows.

(1) A method for producing hepatocytes, comprising differentiating endoderm cells into hepatocytes in the presence of a member of the FGF family, HGF, a member of the IL6 family, and dexamethasone.
(2) The method according to (1), wherein the member of the FGF family is at least one selected from the group consisting of FGF2, FGF1, FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF22, and FGF23.
(3) The method according to (1) or (2), wherein the member of the IL6 family is at least one selected from the group consisting of oncostatin, IL-6, IL-11, IL-27, IL-35, IL-39, LIF, CT-1, CNTF, and CLCF1.
(4) The method according to any one of (1) to (3), wherein the endoderm cells are cells differentiated from pluripotent stem cells.
(5) The method according to any one of (1) to (4), wherein the endoderm cells are derived from human.
(6) Hepatocytes produced by the method according to any one of (1) to (5), wherein the hepatocytes are capable of surviving for 12 days or more while maintaining an albumin secretion ability in ex vivo monolayer culture.
(7) The hepatocytes according to (6), wherein the hepatocytes are capable of surviving for 12 days or more while maintaining functionality as hepatocytes in addition to the albumin secretion ability.
(8) The hepatocytes according to (7), wherein the functionality as hepatocytes, in addition to the albumin secretion ability, is at least one selected from the group consisting of drug metabolism, uptake and release of indocyanine green, glycogen storage, uptake of a low-density lipoprotein, and gene expression.
(9) The hepatocytes according to any one of (6) to (8), wherein the hepatocytes display aging-related characteristics.
(10) The hepatocytes according to (9), wherein the aging-related characteristics is at least one selected from the group consisting of an increased cell volume, expression of aging-related genes, an increased inflammatory response, DNA damage, an increased level of an intracellular reactive oxygen species, an increased level of cellular senescence-associated β-galactosidase, an epigenetic change, a loss of telomere length, and a decline in mitochondrial function.
(11) A method for producing hepatic progenitor cells, the method comprising culturing hepatocytes in the presence of a member of the FGF family.
(12) The method according to (11), wherein the method comprises culturing hepatocytes in the presence of a member of the FGF family and a drug causing histone hyperacetylation.
(13) The method according to (11) or (12), wherein the member of the FGF family is at least one selected from the group consisting of FGF2, FGF1, FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF22, and FGF23.
(14) The method according to (12) or (13), wherein the drug causing histone hyperacetylation is a histone deacetylase inhibitor.
(15) The method according to any one of (12) to (14), wherein the histone deacetylase inhibitor is at least one selected from the group consisting of Trichostatin A, Tacedinaline (CI-994), M344, ITSA-1, Sodium valproate, Sodium 4-phenylbutuyrate, Sodium Butyrate (NaB), valproic acid (VPA), Abexinostat (PCI-24781), Belinostat (PXD101), Citarinostat (ACY-241), Dacinostat (LAQ824), Depudecin, Domatinostat (4SC-202), Droxinostat, Entinostat (MS-275, SNDX-275), Fimepinostat (CUDC-907), Givinostat (ITF2357), Mocetinostat (MGCD0103), Nexturastat A, Panobinostat (LBH-589, NVP-LBH589), Pracinostat (SB939), Quisinostat (JNJ-26481585) 2HC1, Resminostat, Ricolinostat (ACY-1215), Tucidinostat (Chidamide), Vorinostat (SAHA), ACY-738, Apicidin, AR-42, BG45, BML-210, BRD73954, CAY10603, CUDC-101, Curcumin, Depudecin, H1388, HC Toxin, HPOB, LMK-235, MC1568, Oxamflatin, (−)-Parthenolide, PCI-34051, RG2833 (RGFP109), RGFP966, Romidepsin (FK228, Depsipeptide), Santacruzamate A (CAY10683), Scriptaid, SKLB-23bb, Splitomicin, Suberoyl bis-hydroxamic acid, Tasquinimod, TH34, Tinostamustine (EDO-S101), TMP195, TMP269, Tubacin, and Tubastatin A.
(16) The method according to any one of (11) to (15), wherein the hepatocytes are cells differentiated from pluripotent stem cells, cells obtained by passaging cells differentiated from pluripotent stem cells, primary culture cells isolated from a biological tissue, cells obtained by passaging primary culture cells isolated from a biological tissue, or a combination thereof.
(17) The method according to any one of (11) to (16), wherein the hepatocytes are derived from human.
(18) Alpha-fetoprotein (AFP)-negative hepatic progenitor cells produced using the method according to any one of (11) to (17) that have a proliferative ability and a bidirectional differentiation ability into hepatocytes and bile duct epithelial cells.
(19) The hepatic progenitor cells according to (18), wherein the hepatic progenitor cells are capable of differentiating into hepatocytes and/or cholangiocytes.
(20) A method for producing hepatocytes, comprising inducing differentiation of the hepatic progenitor cells according to (18) or (19) into hepatocytes.
(21) A method for producing cholangiocytes, comprising inducing differentiation of the hepatic progenitor cells according to (18) or (19) into cholangiocytes.
(22) A method for suppressing hepatocyte aging using a drug causing histone hyperacetylation.
(23) The method according to (22), wherein the method uses the drug causing histone hyperacetylation in combination with a member of the FGF family.
(24) A hepatocyte anti-aging agent comprising a drug causing histone hyperacetylation as an active ingredient.
(25) The agent according to (24), wherein the drug causing histone hyperacetylation is a histone deacetylase inhibitor.
(26) The agent according to (25), wherein the histone deacetylase inhibitor is at least one selected from the group consisting of Trichostatin A, Tacedinaline (CI-994), M344, ITSA-1, Sodium valproate, Sodium 4-phenylbutuyrate, Sodium Butyrate (NaB), valproic acid (VPA), Abexinostat (PCI-24781), Belinostat (PXD101), Citarinostat (ACY-241), Dacinostat (LAQ824), Depudecin, Domatinostat (4SC-202), Droxinostat, Entinostat (MS-275, SNDX-275), Fimepinostat (CUDC-907), Givinostat (ITF2357), Mocetinostat (MGCD0103), Nexturastat A, Panobinostat (LBH-589, NVP-LBH589), Pracinostat (SB939), Quisinostat (JNJ-26481585) 2HC1, Resminostat, Ricolinostat (ACY-1215), Tucidinostat (Chidamide), Vorinostat (SAHA), ACY-738, Apicidin, AR-42, BG45, BML-210, BRD73954, CAY10603, CUDC-101, Curcumin, Depudecin, H1388, HC Toxin, HPOB, LMK-235, MC1568, Oxamflatin, (−)-Parthenolide, PCI-34051, RG2833 (RGFP109), RGFP966, Romidepsin (FK228, Depsipeptide), Santacruzamate A (CAY10683), Scriptaid, SKLB-23bb, Splitomicin, Suberoyl bis-hydroxamic acid, Tasquinimod, TH34, Tinostamustine (EDO-S101), TMP195, TMP269, Tubacin, and Tubastatin A.
(27) The agent according to any one of (24) to (26), wherein the drug causing histone hyperacetylation is used in combination with a member of the FGF family.
(28) A method for increasing hepatocyte plasticity using a drug causing histone hyperacetylation.
(29) The method according to (28), wherein the method uses the drug causing histone hyperacetylation in combination with a member of the FGF family.
(30) An agent for increasing hepatocyte plasticity, comprising a drug causing histone hyperacetylation as an active ingredient.
(31) The agent according to (30), wherein the drug causing histone hyperacetylation is a histone deacetylase inhibitor.
(32) The agent according to (31), wherein the histone deacetylase inhibitor is at least one selected from the group consisting of Trichostatin A, Tacedinaline (CI-994), M344, ITSA-1, Sodium valproate, Sodium 4-phenylbutuyrate, Sodium Butyrate (NaB), valproic acid (VPA), Abexinostat (PCI-24781), Belinostat (PXD101), Citarinostat (ACY-241), Dacinostat (LAQ824), Depudecin, Domatinostat (4SC-202), Droxinostat, Entinostat (MS-275, SNDX-275), Fimepinostat (CUDC-907), Givinostat (ITF2357), Mocetinostat (MGCD0103), Nexturastat A, Panobinostat (LBH-589, NVP-LBH589), Pracinostat (SB939), Quisinostat (JNJ-26481585) 2HC1, Resminostat, Ricolinostat (ACY-1215), Tucidinostat (Chidamide), Vorinostat (SAHA), ACY-738, Apicidin, AR-42, BG45, BML-210, BRD73954, CAY10603, CUDC-101, Curcumin, Depudecin, H1388, HC Toxin, HPOB, LMK-235, MC1568, Oxamflatin, (−)-Parthenolide, PCI-34051, RG2833 (RGFP109), RGFP966, Romidepsin (FK228, Depsipeptide), Santacruzamate A (CAY10683), Scriptaid, SKLB-23bb, Splitomicin, Suberoyl bis-hydroxamic acid, Tasquinimod, TH34, Tinostamustine (EDO-S101), TMP195, TMP269, Tubacin, and Tubastatin A.
(33) The agent according to any one of (30) to (32), wherein the drug causing histone hyperacetylation is used in combination with a member of the FGF family.
(34) A method for producing a chimeric animal, comprising transplanting into a non-human animal at least one cell selected from the group consisting of the hepatocytes according to (6), the hepatic progenitor cells according to (18), and cells generated by induced differentiation from the hepatic progenitor cells according to (18).
(35) The method for producing the chimeric animal according to (34), comprising transplanting into a non-human animal with liver failure at least one cell selected from the group consisting of the hepatocytes according to (6), the hepatic progenitor cells according to (18), and the cells generated by induced differentiation from the hepatic progenitor cells according to (18).
(36) The method for producing the chimeric animal according to (34) or (35), wherein the transplanted hepatic progenitor cells proliferate and/or differentiate.
(37) The method for producing the chimeric animal according to any one of (34) to (36), wherein liver regeneration is accelerated.
(38) A composition for transplantation, comprising at least one cell selected from the group consisting of the hepatocytes according to (6), the hepatic progenitor cells according to (18), and the cells generated by induced differentiation from the hepatic progenitor cells according to (18).
(39) A kit for a culture medium in induced differentiation of the hepatic progenitor cells according to (18) or (19) into cholangiocytes, the kit including: an agent containing FGF10, retinoic acid, and forskolin for use in the culture medium to be used to induce the differentiation; and instructions of the use of the agent in the culture medium to be used to induce the differentiation.
(40) A medium comprising FGF10, retinoic acid, and forskolin for inducing differentiation of the hepatic progenitor cells according to (18) or (19) into cholangiocytes.
(41) A liver regeneration accelerator, comprising a drug causing histone hyperacetylation as an active ingredient.
(42) A method for accelerating liver regeneration, comprising administering to a subject a pharmaceutically effective amount of a drug causing histone hyperacetylation.

### Advantageous Effects of Invention

The present invention has attained hepatocytes serving as a model of senescence.

The present invention has made it possible to induce plasticity in hepatocytes. The hepatocytes induced to have plasticity (hepatic progenitor cells) can differentiate into hepatocytes or cholangiocytes.

The present invention has made it possible to induce plasticity in old hepatocytes as well.

The present specification includes part or all of the contents as disclosed in the specification and/or drawings of Japanese Patent Application No. 2019-177843, which is the basis of the priority of the present application.

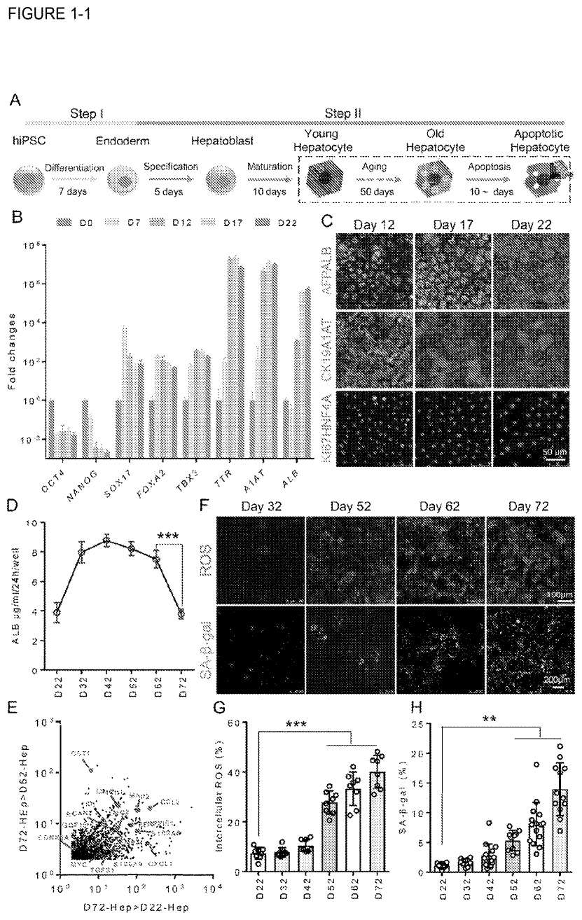

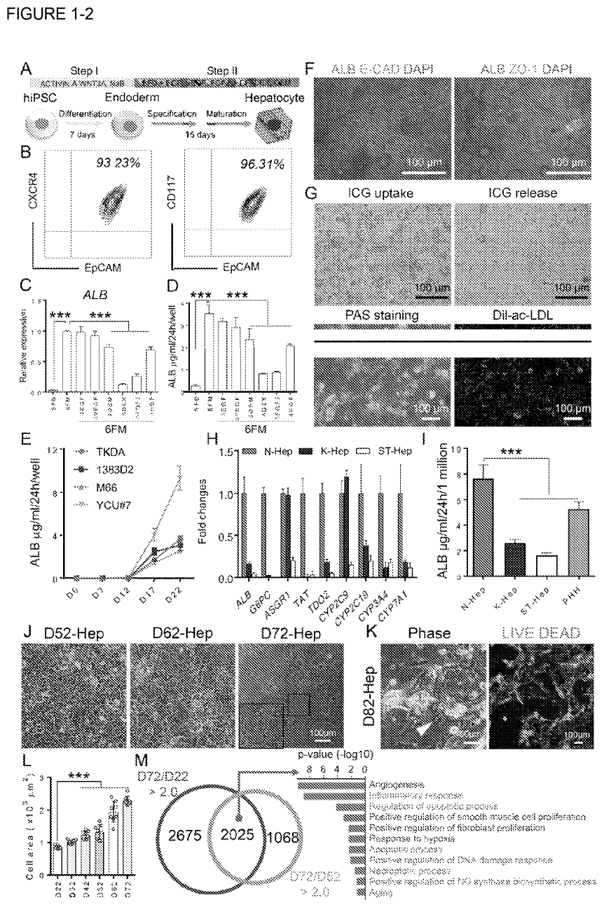

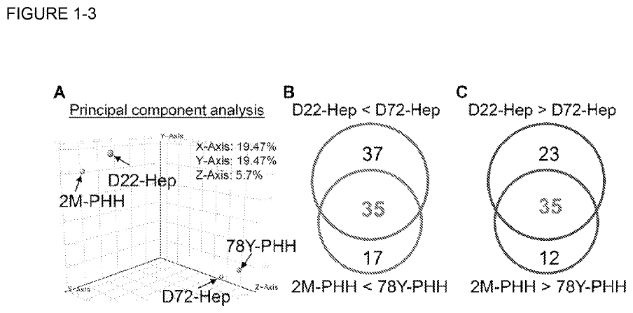

### BRIEF DESCRIPTION OF THE DRAWINGS

[FIG. 1-1] Recapitulation of the Aging Process by Means of human induced pluripotent stem cell-derived hepatocytes (hiPSC-Hep)

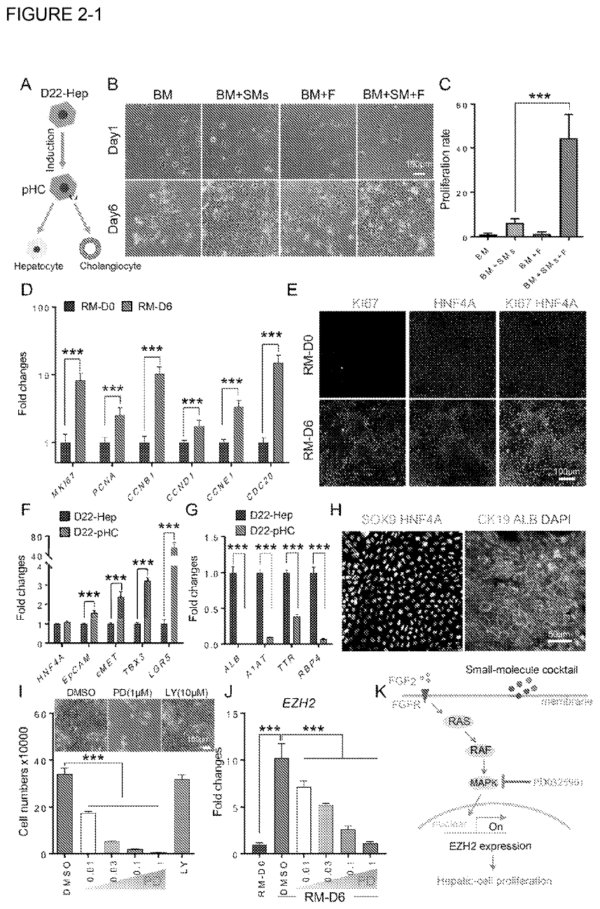

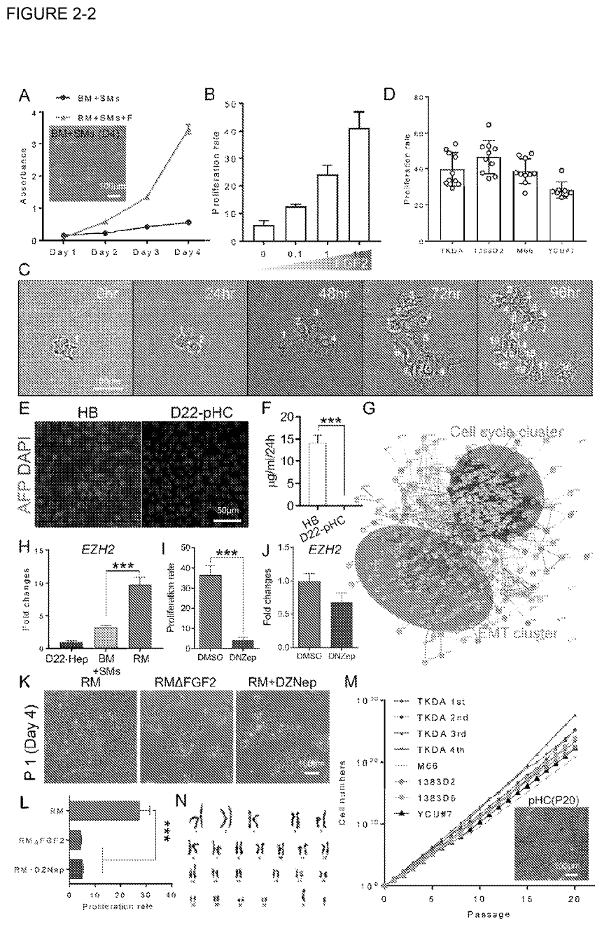

(A) Schematic representation of hepatic specification, maturation, and aging via the new strategy based on hiPSC-Hep.
(B) Q-PCR analysis of expression of lineage-related genes during the progression from hiPSC to hepatocyte: OCT4 and NANOG (pluripotent cell), SOX17 and FOXA2 (endoderm), TBX3 and TTR (hepatoblast), and A1AT and ALB (hepatocyte).
(C) Immunofluorescence staining of ALB, AFP, CK19, A1AT, HNF4A, and KI67.
(D) An enzyme-linked immunosorbent assay (ELISA)-based analysis of ALB secretion from N-Heps (N-Heps are hepatocytes derived from hiPSCs that were prepared by the inventors by the method described in Example) from day 22 (D22) to day 72 (D72).
(E) Transcriptome analysis shows upregulation of some genes in D72-Hep (hepatocyte after 72 days in the whole process, which were differentiated from hiPSCs and passaged, corresponding to “Old Hepatocyte” in FIG. 1-1A) compared with D22-Hep (hepatocyte after 22 days in the whole process, which were differentiated from hiPSCs and passaged, corresponding to “Yong Hepatocyte” in FIG. 1-1A) and D52-Hep (hepatocyte after 52 days in the whole process, which were differentiated from hiPSCs and passaged); big points representing gene names: aging-related genes.
(F) Images of intracellular ROS (upper images, red) and SA-β-gal staining (lower images, green) in N-Hep from D32 to D72.
(G) Quantification of the percentage of intracellular-ROS-positive cells among N-Hep from D22 to D72.
(H) Quantification of SA-β-gal-positive cells among N-Hep from D22 to D72. See also FIGS. 1-2 and 1-3.
[FIG. 1-2] Development of a two-step protocol to generate hiPSC-Hep, and characteristics of hiPSC-Hep's function and alterations during aging (related to FIG. 1-1)
(A) Schematic diagram of the 2-step hepatocyte differentiation from hiPSC.
(B) A representative flow cytometry profile showing the expression of CXCR4, CD117, and EpCAM in hiPSC-derived definitive endoderm.
(C) Q-PCR analysis of ALB expression in lineages differentiated with designated mediums in step II; “Δ” means “without”; SFD: serum-free defined medium; 6FM: SFD with six selected factors, OSM, Oncostatin M; DEX: dexamethasone.
(D) ELISA-based assays of ALB secretion in lineages cultured with designated mediums in step II.
(E) ELISA-based dynamic assays of ALB secretion during hepatocyte differentiation from multiple donor derived hiPSC clones.
(F) Immunostaining of N-Hep with ALB (red), E-cadherin (E-CAD) (green), and zona occludens (ZO-1) (green).
(G) Analyses of indocyanine green (ICG) uptake and release (green), detection of glycogen storage by periodic acid-Schiff staining (purple), and examination of low-density lipoprotein intake with Dil-labeled acetyl LDL (DiI-ac-LDL) (red) in N-Hep.
(H) Q-PCR analysis of expression of hepatic-related genes in N-Hep, K-Hep, and ST-Hep (the respective hiPSC-Heps obtained by the conventional protocols shown below, with the designations K-Hep, and ST-Hep using the initials of the researchers; Kajiwara et al, 2012 and Si-Tayeb et al., 2010).
(I) ELISA-based assays of ALB secretion from N-Hep, K-Hep, ST-Hep, and PHH.
(J) Cellular morphology of D52-Hep, D62-Hep, and D72-Hep; square box: multinucleated cells.
(K) Cellular morphology (left) and LIVE/DEAD staining (right) of D82-Hep; white arrowhead: multinucleated cells.
(L) Quantification of average cellular sizes in N-Hep from D22 to D72.
(M) Gene ontology analysis for enriched pathways and molecular function in D72-Hep.
[FIG. 1-3] Comparison of in vitro and in vivo hepatocyte aging processes
(A) Principal component analysis provided a three-dimensional graphical representation of the gene expression clustering among D22-Hep, D72-Hep, young PHH (2 months old, 2M-PHH) and elderly PHH (78 years old, 78Y-PHH).
(B) D72-Hep and 78Y-PHH are compared with D22-Hep and 2M-PHH, respectively, by KEGG pathway analysis of genes upregulated with aging.
(C) D72-Hep and 78Y-PHH are compared with D22-Hep and 2M-PHH, respectively, by KEGG pathway analysis of genes downregulated with aging.

### [FIG. 2-1] FGF2 Enhances Proliferation Induced in D22-Hep

(A) The schematic overview of characterization of plasticity in D22-Hep.
(B) Phase contrast images (on D1 and D6) of D22-Hep cultured in the BM, BM+SMs, BM+FGF2 (F), or BM+SMs+F (the latter is the reprogramming medium; RM). BM is SFD medium containing 10 ng/mL EGF and 20 ng/mL HGF (described in WO2016093222); BM+SMs is BM medium containing 10 μM Y-27632, 0.5 μM A83-01 and 3 μM CHIR99021.
(C) Quantification of cell numbers after 6 days of plasticity induction; the values were normalized to the cell number on Dl.
(D) Q-PCR measurement of expression of cell cycle-related genes in cells before cultivation (D0) and after 6 days of cultivation in RM. These time points are presented in the diagram as RM-D0 and RM-D6, respectively.
(E) Immunofluorescence staining of KI67 and HNF4A on cells of groups RM-D0 and RM-D6.
(F) Q-PCR measurement of expression of hepatic-progenitor/stem cell—related genes HNF4A, EpCAM, C-MET, TBX3, and LGR5 in D22-Hep and D22-pHC (hepatic progenitors that the inventors induced by the method described in Example).
(G) Q-PCR analysis of expression of hepatic functional genes ALB, A1AT, TTR, and RBP4 in D22-Hep and D22-pHC.

### (H) Immunostaining of D22-pHC for HNF4A, SOX9, CK19, and ALB.

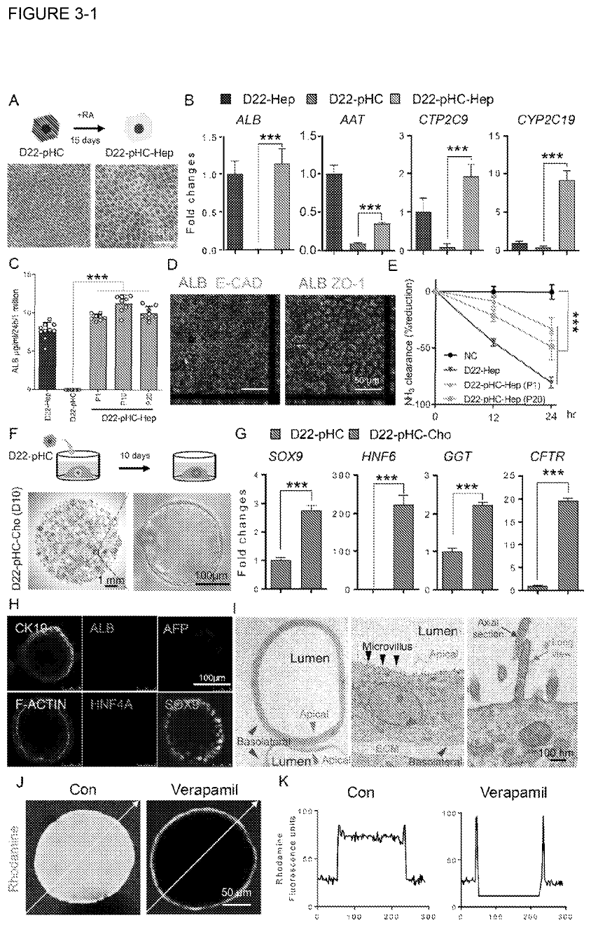

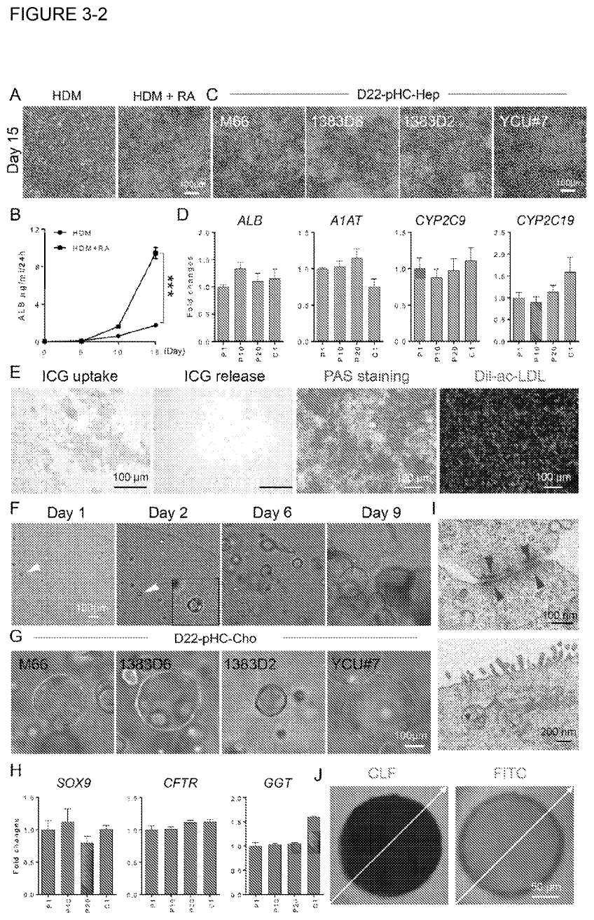

(I) Analysis of cellular morphology after 6 days of treatment with RM plus DMSO, PD0325901 (PD, 1 μM), or LY2940002 (LY, 10 μM) and quantification of cell numbers after 6 days of incubation in RM plus DMSO, PD0325901 (0.01, 0.03, 0.1, or 1 μM), or LY290002 (10 μM); the values were normalized to the cell number on Dl.
(J) The transcription of EZH2 was measured by Q-PCR under the indicated conditions.
(K) The scheme illustrating how the FGF2—MAPK-EZH2 axis promotes hepatocyte proliferation. See also FIG. 2-2.
[FIG. 2-2] The FGF2-activated MAPK-EZH2 axis promotes the induction of hepatocyte plasticity (related to FIG. 2-1).
(A) Cell Counting Kit-8-based analyses of the proliferative capacity of cells sub-cultured and reprogramed in BM+SMs and BM+SMs+F, and phase contrast images on day 4 of cells sub-cultured in BM+SMs.
(B) Cell numbers counted after 6 days of treatment of BM+SMs with FGF2 (0, 0.1, 1, or 10 ng/ml); the values were normalized to cell numbers on Day 1.
(C) Time-lapse imaging analyses of the induced stem cell proliferation at the single cell level.
(D) Induction of hepatocyte proliferation with RM from D22-Heps derived from different hiPSC clones.
(E) Immunofluorescence analysis of AFP on hiPSC-hepatoblast (HB) and proliferative hepatic cells (D22-pHC).
(F) ELISA-based assays of AFP secretion from HB and D22-pHC.
(G) STRING interaction network of enriched genes in D22-pHC compared with D22-Hep.
(H) Q-PCR analysis of EZH2 expression in D22-Hep, cells reprogrammed in BM+SMs, and cells reprogrammed in RM.
(I) Quantification of cell numbers in RM with DMSO or 3-deazaneplanocin A hydrochloride (DZNep) (0.1 μM) on day 6; the values were normalized to cell numbers on Day 1.
(J) Q-PCR analysis of EZH2 expression on day 6 in cells cultured in RM with DMSO or DZNep (0.1 μM).
(K) Phase contrast images on day 4 of reseeding of D22-pHC in RM, RMAFGF2 (RM deprived of FGF2), and RM+DNZep (0.1 μM).
(L) Cell numbers on day 4 of D22-pHC cultured in RM, RMΔFGF2, and RM+DNZep (0.1 μM); the values were normalized with cell numbers on Day 1.
(M) Prolonged growth-period curves of D22-pHC (from P0 to P20). These results were obtained from five different hiPSC (TKDA)-derived pHCs, and phase contrast image of pHC at passage 20.
(N) Representative karyotype images of D22-pHC (TKDA) at passage 18.
[FIG. 3-1] D22-pHC with the Bipotential Property Differentiate into Hepatocyte and Cholangiocyte
(A) Top: The schedule of hepatic differentiation of D22-pHC. Bottom: Phase contrast images of D22-pHC (left) and D22-pHC-derived hepatocyte (D22-pHC-Hep, right).
(B) Q-PCR analysis of expression of ALB, A1AT, CYP2C9, and CYP2C19 in D22-Hep, D22-pHC, and D22-pHC-Hep.
(C) ELISA-based analyses of ALB secretion from D22-Hep, D22-pHC, D22-pHC-Hep (P1) (D22-pHC-derived hepatocyte at passage 1), D22-pHC-Hep (P10) (D22-pHC-derived hepatocyte at passage 10), and D22-pHC-Hep (P20) (pHC-derived hepatocyte at passage 20).
(D) Immunofluorescence assays of ALB, E-CAD, and ZO-1 in D22-pHC-Hep.
(E) Ammonia clearance in D22-pHC-Hep (P1), D22-pHC-Hep (P20), and D22-Hep; the no-cell (NC) group served as a negative control.
(F) Top: The schedule of cholangiocytic differentiation of D22-pHC. Bottom: A macroscopic image of D22-pHC-derived cholangiocyte (D22-pHC-Cho) (left), and a macroscopic image of a single cholangiocyte cyst (right).
(G) Q-PCR measurement of expression of SOX9, HNF6, GGT, and CFTR in D22-pHCs and D22-pHC-Cho.
(H) Immunofluorescence staining of CK19, ALB, AFP, F-ACTIN, HNF4A, and SOX9 in D22-pHC-Cho.
(I) Transmission electron microscopy images of cholangiocyte cysts: the integrated structure of D22-pHC-Cho cysts with a lumen, an apical (red arrowhead) and basolateral (blue arrowhead) plasma membrane, and microvilli (black arrowhead); a partial longitudinal section (red arrow) and axial cross-section (blue arrows) of primary cilia.
(J) Representative images of Rhodamine 123 accumulation in the lumen of a D22-pHC-Cho cyst in the absence and presence of verapamil.
(K) Calculation of fluorescence intensity along the white line in the images of panel (J) above. See also FIG. 3-2.
[FIG. 3-2] Characterization of D22-pHC-derived hepatocytes and cholangiocytes (related to FIG. 3-1).
(A) Phase contrast images of D22-pHC after 15 days of culturing in HDM and HDM+RA, respectively.
(B) ELISA-based dynamic assays of ALB secreted in HDM and HDM+RA during hepatocyte differentiation. HDM consisted of SFD with 10 ng/mL FGF2, 20 ng/mL HGF, 10 ng/mL OSM, 100 nM dexamethasone and 10 mM nicotinamide.
(C) Images of hepatocytes (D22-pHC-Hep) differentiated from multiple donor hiPSC clone derived D22-pHCs.
(D) Q-PCR analysis of expression of ALB, A1AT, CYP2C9, and CYP2C19 in hepatocytes differentiated from D22-pHCs at different passages (P1, P10, and P20), and clonal D22-pHC (C1) at P5.
(E) Analyses of ICG uptake and release (green), detection of glycogen storage by periodic acid-Schiff staining (purple), and examination of low-density lipoprotein intake with Dil-ac-LDL (red) in D22-pHC-Hep.
(F) Time-lapse imaging analyses of cholangiocyte cyst formation, arrowhead: a single D22-pHC generated a ring-like structure.
(G) Microscopic images of cholangiocyte (D22-pHC-Cho) derived from multiple donor hiPSC clone derived D22-pHCs.
(H) Q-PCR analysis of expression of SOX9, GGT, and CFTR in cholangiocytes differentiated from D22-pHC at different passages (P1, P10, and P20), and clonal D22-pHC (C1) at P5.
(I) Transmission electron microscopy images of tight junctions (blue arrowhead) (upper), and multivesicular bodies (red arrowhead) (lower) in cholangiocytes.
(J) Representative images of active export of the fluorescent bile acid CLF from the cyst lumen, and the FITC (fluorescein isothiocyanate) load as a control.

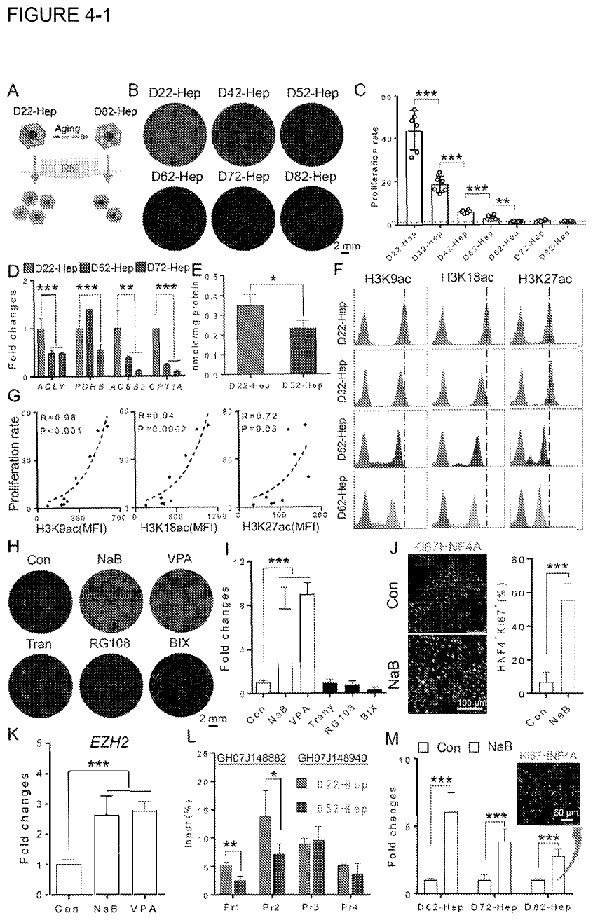

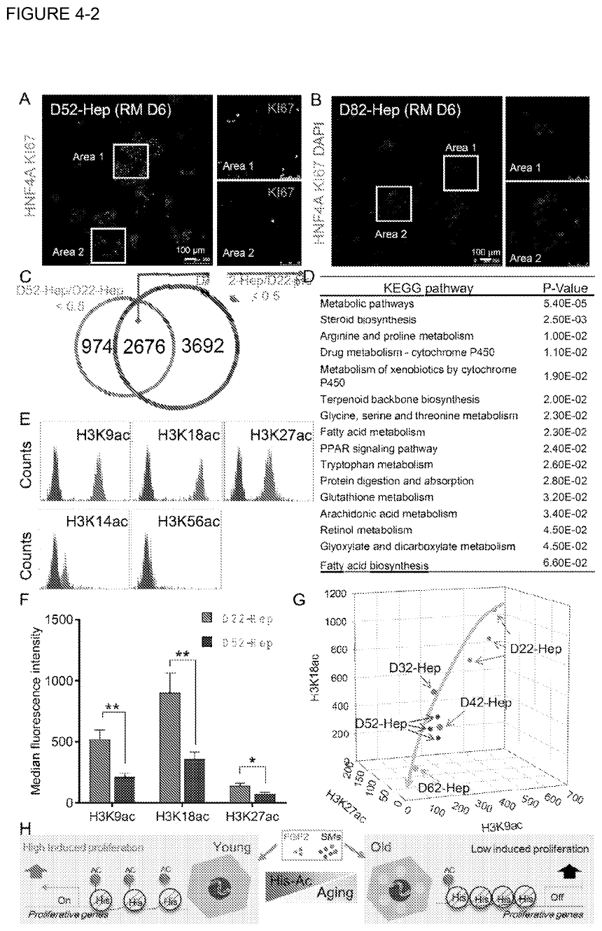

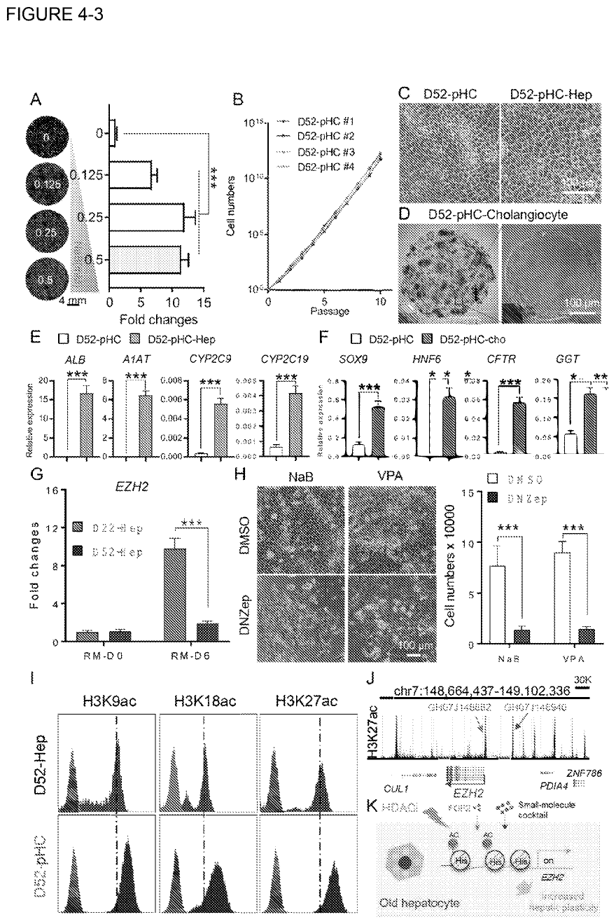

### [FIG. 4-1] Age-related Histone Hypoacetylation Hampers the Induced Stem Cell Proliferation

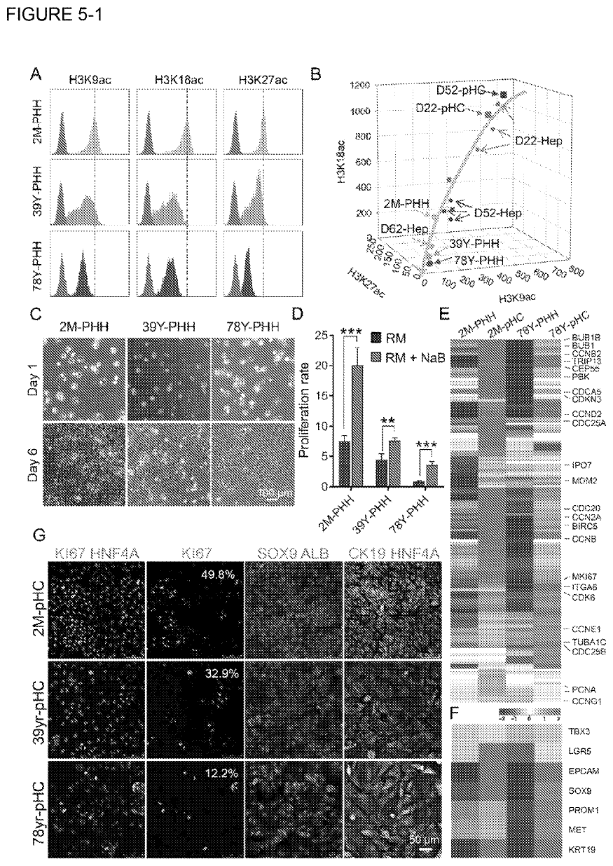

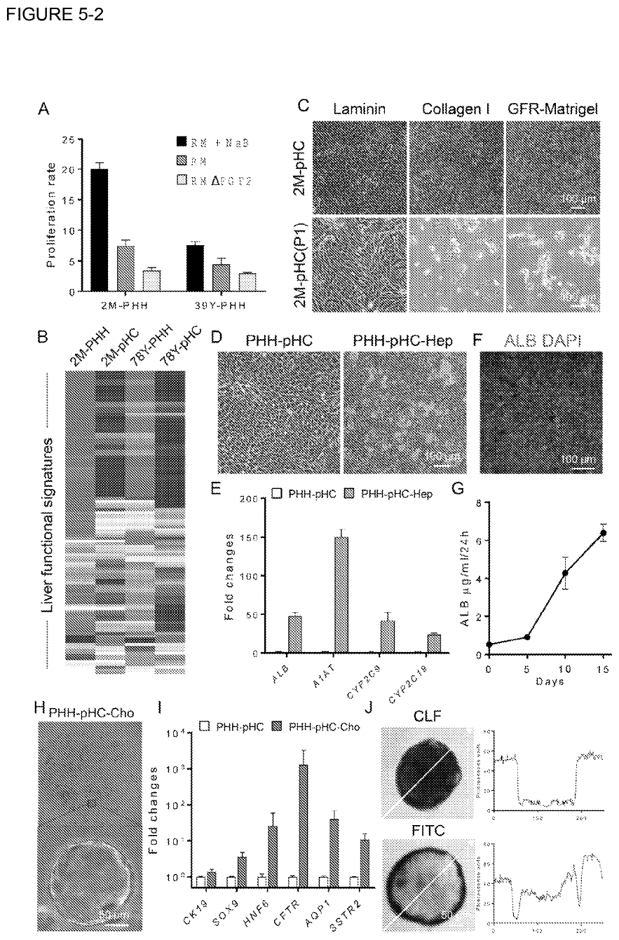

(A) Schematic analyses of aging-associated plasticity of N-Hep.
(B) Hoechst staining-based images of proliferative cells derived from N-Heps after 6 days of cultivation in RM.
(C) Quantification of the cells derived from N-Heps after 6 days of culture in RM; the data were normalized to the cell number on Dl.
(D) The expression of acetyl-CoA synthesis related genes (ACLY, PDHB, ACSS2 and CPT1A) in D22-Hep, D52-Hep, and D72-Hep was evaluated by Q-PCR.
(E) Analysis of intracellular acetyl-CoA levels in D22-Hep and D52-Hep.
(F) Fluorescence-activated cell sorting-based analyses of the amounts of H3K9ac, H3K18ac, and H3K27ac in D22-Hep, D32-Hep, D52-Hep, and D62-Hep.
(G) Correlation of the levels of H3K9ac (left), H3K18ac (middle), and H3K27ac (right) with the induced proliferation rate of N-Heps as accompanying the aging.
(H) Hoechst staining-based-image analyses of the reprogrammed cells derived from D52-Hep cultured in RM with sodium butyrate (NaB), valproic acid (VPA), tranylcypromine (Trany, histone demethylase inhibitor), RG108 (DNA methyltransferase inhibitor) or BIX01294 (BIX, histone methyltransferase inhibitor) added on D8. The group cultured with only RM was a control (Con).
(I) Quantification of the fold change of proliferation rates after addition of NaB, VPA, Trany, RG108, or BIX.
(J) Immunofluorescence assays of HNF4A (Red) and KI67 (Green) in reprogrammed cells derived from D52-Hep cultured in RM (Con) or RM+NaB (with NaB added on D8); the percentage of HNF4A+KI67+ cells is shown in the right-hand chart.
(K) Q-PCR analysis of EZH2 expression in reprogrammed cells derived from D52-Hep cultured in RM (Con), RM+NaB (NaB), or RM+VPA (VPA).
(L) Chromatin Immunoprecipitation coupled with PCR (ChIP-PCR) detection of acetylated-histone binding within the EZH2 enhancers in D22-Hep and D52-Hep by means of four primer sets (Pr).
(M) Quantitation of the promotion, as driven by NaB (added on D8), of hepatocyte plasticity induction in D62-Hep, D72-Hep, and D82-Hep. All the data were normalized to the corresponding RM-only group. Immunofluorescence staining of HNF4A (red) and KI67 (green) in the reprogrammed cells derived from D82-Hes under the RM+NaB condition. See also FIGS. 4-2 and 4-3.
[FIG. 4-2] Histone hypoacetylation impairs plasticity induction in aged hepatocytes (related to FIG. 4-1).
(A) Immunofluorescence analysis of HNF4A (Red) and KI67 (Green) in reprogrammed D52-Hep on day 6.
(B) Immunofluorescence analysis of HNF4A (Red) and KI67 (Green) in reprogrammed D82-Hep on day 6.
(C) Venn diagram displaying the total and the common number of the genes in D52-Hep and D72-Hep that were downregulated as compared with D22-Hep (<0.5-fold).
(D) KEGG pathway analysis of the genes in D52-Hep and D72-Hep that were both downregulated as compared with D22-Hep (<0.5-fold).
(E) FACS-based analyses of H3K9ac, H3K18ac, H3K27ac, H3K14ac, and H3K56ac in D22-Hep.
(F) Comparison of H3K9ac, H3K18ac, and H3K27ac levels in D22-Hep and D52-Hep.
(G) Quantification of the median fluorescence intensity (MFI) of H3K9ac, H3K18ac, and H3K27ac in N-Heps during aging.
(H) Schematic illustration of histone hypoacetylation mediated impairment of the induction of hepatocyte plasticity in old hiPSC-Heps.
[FIG. 4-3] HDAC inhibitors improve the plasticity of D52-Hep (related to FIG. 4-1).
(A) Left: Hoechst staining-based image analyses on day 8 of cells reprogramed from D52-Hep in RM with NaB (0, 0.125, 0.25, or 0.5 mM); right: quantification of improved D52-pHC cell proliferation in RM with NaB (0, 0.125, 0.25, or 0.5 mM), all values were normalized to those of the NaB=0 group.
(B) Prolonged growth-period curves of D52-pHC (from P0 to P10).
(C) Phase contrast images of D52-pHC (left) and D52-pHC-derived hepatocytes (D52-pHC-Hep, right).
(D) A macroscopic image of D52-pHC-derived cholangiocytes (D52-pHC-Cho, left), and macroscopic image of a single cholangiocyte cyst (right).
(E) Q-PCR analysis of expression of ALB, A1AT, CYP2C9, and CYP2C19 in D52-pHC and D52-pHC-Hep.
(F) Q-PCR analysis of expression of SOX9, HNF6, CFTR, and GGT in D52-pHC and D52-pHC-Cho.
(G) Analysis of EZH2 transcription in D22-Hep and D52-Hep before RM treatment (RM-D0) and thereafter (RM-D6).
(H) Images of cells reprogrammed from D52-Hep in RM+NaB (NaB) and RM+VPA (VPA) with or without DNZep; cell numbers were counted on day 8 and normalized to the cell number on day 1.
(I) FACS-based analyses of the levels of H3K9ac, H3K18ac, and H3K27ac in D52-Hep and D52-pHC.
(J) Analysis of histone acetylation binding sites at the EZH2 promoter from the ENCODE dataset; GH07J148882 and GH07J148940 were the two highest scored EZH2 enhancers.
(K) Schematic diagram of HDAC inhibitors (HDACi) improving the hepatocyte plasticity of old hepatocytes.
[FIG. 5-1] Induction of pHC Formation from PHHs
(A) Fluorescence-activated cell sorting-based analyses of H3K9ac, H3K18ac, and H3K27ac in primary human hepatocytes (PHHs derived from 2 months old (2M-PHHs), 39 years old (39Y-PHHs), and 78 years old (78Y-PHHs) donors).
(B) Quantification of the median fluorescence intensity of H3K9ac, H3K18ac, and H3K27ac in PHHs, N-Heps, and pHCs.
(C) Phase contrast images on D1 and D6 of PHHs cultured in RM+NaB.
(D) Quantification of proliferation induced in PHHs incubated in a medium consisting of RM or RM+NaB; the data were normalized to each cell number on Dl.
(E) Microarray analyses of mRNA expression of cell cycle-related genes in PHHs and PHH-pHCs.
(F) Microarray analyses of mRNA expression of hepatic progenitor markers in PHHs and PHH-pHCs.
(G) Immunofluorescence staining of KI67, HNF4A, SOX9, ALB, and CK19 on PHH-pHCs. See also FIG. 5-2
[FIG. 5-2] Characteristics of PHH plasticity (related to FIG. 5-1).
(A) Quantitation of the improvement of PHH-pHC proliferation by NaB and FGF2 with the plasticity of 2M-PHH and 39Y-PHH being induced on a Laminin 511-coated plate; the values were normalized with cell numbers in each RM+NaB group.
(B) Comparison of liver functional signatures between PHHs and PHH-pHCs.
(C) Images of the passage of 2M-PHH-pHC on plates coated with different matrices.
(D) Phase contrast images of PHH-pHC (left) and PHH-pHC-derived hepatocytes.
(E) Q-PCR analysis of expression of ALB, A1AT, CYP2C9, and CYP2C19 in PHH-pHC and PHH-pHC-Hep.

### (F) Immunostaining of ALB in PHH-pHC-Hep.

(G) ELISA-based assays of ALB secretion during hepatocyte differentiation from PHH-pHC to PHH-pHC-Hep.
(H) The macroscopic images of PHH-pHC-derived cholangiocytes (PHH-pHC-Cho, upper), and a macroscopic image of a single cholangiocyte cyst (lower).
(I) Q-PCR analysis of expression of CK19, SOX9, HNF6, CFTR, AQP1, and SSTR2 in PHH-pHC and PHH-pHC-Cho; the values were normalized to the gene expression in PHH-pHC.
(J) Representative images of active CLF export from the cyst lumen of PHH-pHC-Cho, and the FITC load as a control. The fluorescence intensity was calculated along the white arrows in the left images.

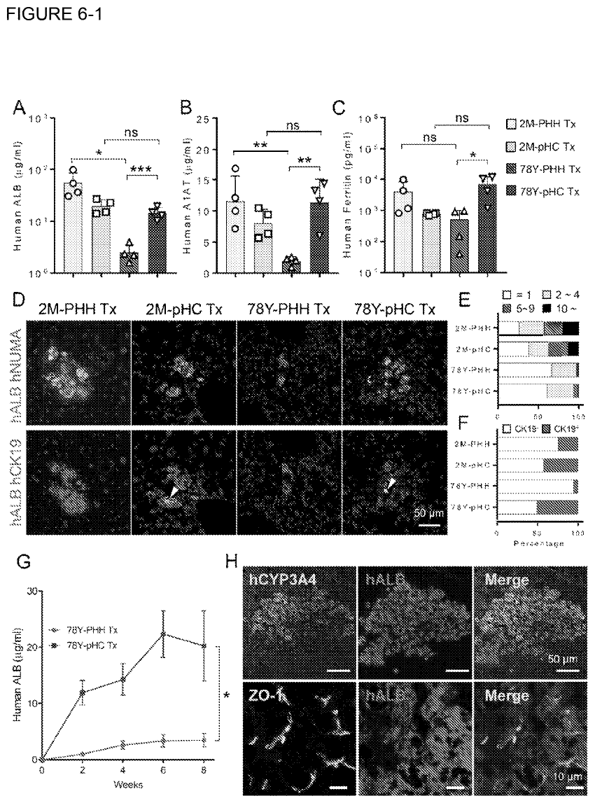

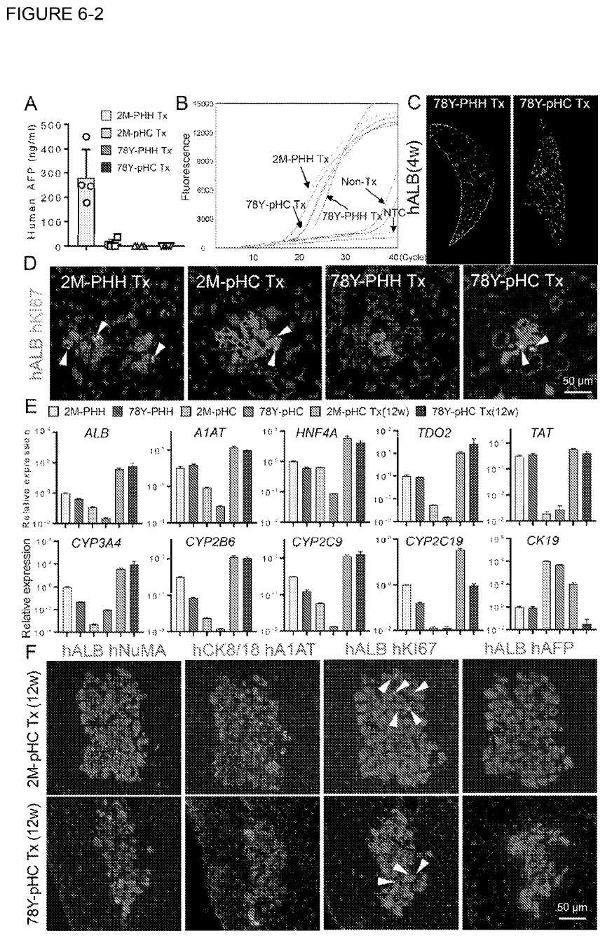

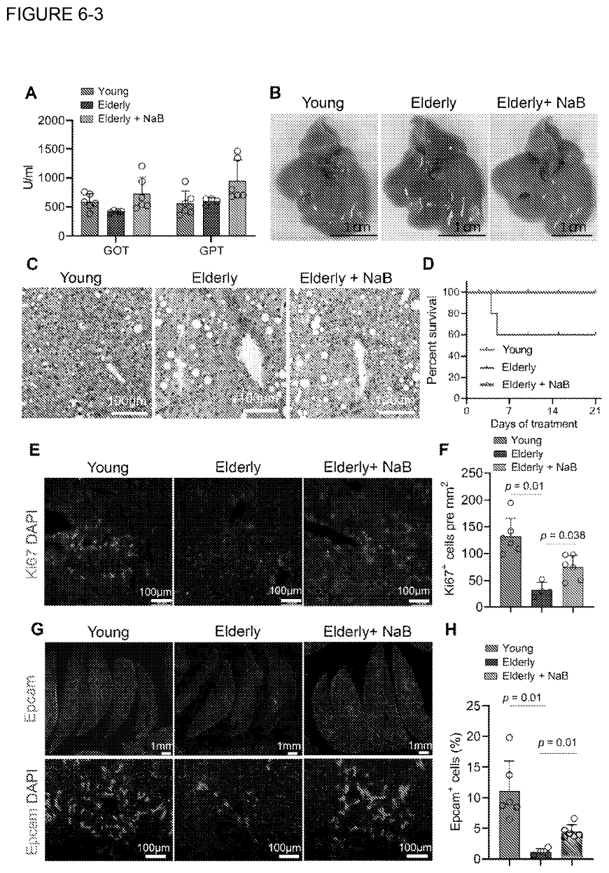

### [FIG. 6-1] Engraftment of PHH-pHCs in the Mouse Model of Liver Injury

(A) ELISA-based analyses of hALB secretion in PHH-Tx mice and PHH-pHC-Tx mice at 4 weeks after transplantation.
(B) ELISA-based analyses of hA1AT secretion in PHH-Tx mice and PHH-pHC-Tx mice at 4 weeks after transplantation.
(C) ELISA-based analyses of hFerritin secretion in PHH-Tx mice and PHH-pHC-Tx mice at 4 weeks after transplantation.
(D) Immunofluorescence assays of hALB, hNUMA, and hCK19 in the liver of PHH-Tx mice and PHH-pHC-Tx mice at 4 weeks after transplantation.
(E) Quantification of engraftment size in the liver of PHH-Tx mice and PHH-pHC-Tx mice at 4 weeks after transplantation. (1, 2-4, 5-9, and 10— in the caption indicate the number of cells in the cluster.)
(F) Quantification of hALB+hCK19+ and hALB+hCK19− engraftment in the liver of PHH-Tx mice and PHH-pHC-Tx mice at 4 weeks after transplantation.
(G) ELISA-based dynamic analyses of human ALB expression in 78Y-PHH-Tx mice and 78Y-pHC-Tx mice after transplantation (n=3 mice per group).
(H) Immunofluorescence assays of human ALB, CYP3A4, and ZO-1 in the liver of 78Y-pHC-Tx mice at 12 weeks after transplantation.
[FIG. 6-2] Engraftment of PHH-pHC in a liver injury mouse model (related to FIG. 6-1).
(A) ELISA-based assays of AFP secretion in PHH Tx mice and PHH-pHC Tx mice at 4 weeks after transplantation.
(B) Amplification curves of human ALB in PHH Tx mice and PHH-pHC Tx mice.
Determined by PCR based on human-specific markers; Non-Tx: mice transplanted with only PBS; NTC: control without template (no template control).
(C) Comparison of ALB+ engraftment in 78Y-PHH Tx mice and 78Y-pHC Tx mice at 4 weeks after transplantation.
(D) Immunofluorescence analysis of ALB and KI67 in PHH Tx mice and PHH-pHC Tx mice at 4 weeks after transplantation.
(E) Human-specific primer-based Q-PCR analyses of the maturation of PHH-pHCs at 12 weeks after transplantation.
(F) Immunofluorescence analysis of hALB, hNuMA, hA1AT, hCK8/18. hKI67 and hAFP in PHH-pHC Tx mice at 12 weeks after transplantation.
[FIG. 6-3] HDAC inhibitor improves the induction of pHCs in the elderly model.
(A) The detection of GOT and GPT in young mice (n=6), elderly mice (Elderly, n=4) and elderly mice with NaB treatment (Elderly+NaB, n=6) after 21 days of feeding on a choline-deficient, ethionine-supplemented (CDE) diet.
(B) Macroscopic images of mouse livers after 21 days of feeding on a CDE diet.
(C) Hematoxylin & Eosin staining of mouse livers after 21 days of feeding on a CDE diet.
(D) Kaplan-Meier survival curves of mice fed on a CDE diet.
(E,F) Immunofluorescence staining (E) and Quantification (F) of Ki67 in liver sections of CDE fed mice.
(G,H) Immunofluorescence staining (G) and Quantification (H) of Epcam (Epithelial cell adhesion molecule) in liver sections of CDE fed mice.

### DESCRIPTION OF EMBODIMENTS

Hereinafter, the present invention will be described in detail.

### 1. A Novel Method for Inducing Hepatocytes and Production of a Model of Aged Hepatocyte

The present invention provides a method for producing hepatocytes that includes differentiating endoderm cells into hepatocytes in the presence of a member of the FGF family, HGF, a member of the IL6 family, and dexamethasone. The method of the present invention is a simple and convenient method for producing hepatocytes that can differentiate endoderm cells into hepatocytes in one step.

Fibroblast growth factors (FGF) are a class of growth factors and are involved in angiogenesis, wound healing, and embryogenesis. In the present invention, examples of the member of the FGF family include FGF2, FGF1, FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF22, FGF23, and combinations thereof, with FGF2 being preferred.

Hepatocyte growth factor (HGF), a cytokine which was discovered as a substance promoting the proliferation of hepatocytes in the blood of a partially hepatectomized rat and purified as a factor strongly promoting the proliferation of primary culture hepatocytes, is a promising candidate for a liver-regenerating factor that supports vigorous regeneration of a liver. HGF is a large growth factor, and HGF mRNA is a single-stranded precursor for 728 amino acid residues that is to be translated as Pro HGF (94 kD) with no biological activity; Pro HGF, upon having its N-terminal signal peptide removed and being secreted extracellularly, undergoes specific cleavage (processing) between Arg and Val by proteases such as HGF activator (HGFA), a coagulation factor (XIa), urokinase (u-PA), and matriptase, and then is produced as Mature (active) HGF with a heterodimeric structure in which the alpha (62 kD) and beta (34 kD) chains linked by a disulfide bond. HGF promotes the proliferation of primary culture rat hepatocytes, and also promotes the proliferation of various epithelial cells, endothelial cells, and some mesenchymal cells as well. In addition, HGF has a wide variety of biological activities that play a role in regeneration and protection of tissues and organs, such as promotion of cell proliferation, promotion of cell dispersion or cell motility, anti-apoptosis (cell death), induction of morphogenesis (such as tube formation), angiogenesis, antifibrotic action, and regulation of immune response. A receptor for HGF is a product of proto-oncogene c-met, which has tyrosine kinase activity, and the diverse biological effects of HGF are exerted through c-Met. During liver regeneration, increased HGF gene expression in the liver, spleen, and lung, as well as increased levels of HGF in the blood and liver, precede the induction of hepatic DNA synthesis, and liver regeneration is suppressed by administration of anti-HGF antibodies. HGF-producing cells are mainly produced and secreted by mesenchymal cells such as fibroblasts, fat-containing cells, neutrophils, macrophages, and vascular endothelial cells, and they act on normal epithelial cells or cancer cells in a paracrine manner. On the other hand, it has been reported that cancer cells also produce HGF and have an autocrine effect, i.e., activates their own c-MET. The cells that have been reported to produce HGF in the liver are stellate cells of a liver sinusoid and sinusoidal endothelial cells. Taking liver regeneration as an example, various acute injuries such as partial hepatectomy, hepatitis, and hepatic ischemia are accompanied by increased expression of HGF not only in the damaged liver but also in intact organs such as the remote lungs and kidneys, whereupon the HGF level in the blood is increased. In fact, when antibodies that neutralize the activity of HGF are administered to rats or mice suffering liver damage, they undergo a significant expansion of the liver damage and failure in liver regeneration. Similar results have been observed in other organ damages, showing that HGF is an endogenous factor responsible for regeneration and protection of various tissues and organs, including the liver, kidney, lung, cardiovascular system, and nervous system. HGF production in cultured cells is induced by PKC activators, PKA activators, cAMP-elevating agents, various growth factors, inflammatory cytokines (such as IL-1 and TNF-α), or the like and is suppressed by TGF-β, glucocorticoids, the active form of vitamin D, retinoic acid, or the like. There have been many reports showing that HGF, by virtue of its potent proliferation-promoting effect on various cells, can be applied as a therapeutic agent for various intractable organ diseases such as cirrhotic liver, chronic kidney failure, pulmonary fibrosis, myocardial infarction, or arteriosclerosis obliterans. In addition, serum and plasma HGF quantitative ELISA kits have been used for prediction of fulminant hepatitis.

Interleukin-6 (IL-6) is a lectin produced by cells such as T cells and macrophages and is one of the cytokines that regulate humoral immunity. In the present invention, examples of the member of the IL6 family include oncostatin (OSM), IL-6, IL-11, IL-27, IL-35, IL-39, LIF, CT-1, CNTF, CLCF1, and combinations thereof, with oncostatin being preferred.

Dexamethasone is a synthetic adrenal cortex hormone preparation that exerts its anti-inflammatory action through the same mechanism as natural glucocorticoids, and is used in acute inflammation, chronic inflammation, autoimmune diseases, allergic diseases, etc.

Endoderm cells are cells which express endoderm markers, and which can differentiate into various endodermal lineage cells (such as cells of the lung, liver, pancreas, stomach, small intestine, and large intestine).

Examples of the endoderm markers include SOX17, FOXA2, CXCR4, EpCAM, C-KIT, and CER1; examples of the endoderm cells include cells of the lung, liver, pancreas, stomach, small intestine, and large intestine.

The endoderm cells can be obtained from induction of differentiation of pluripotent stem cells. Examples of the pluripotent stem cells include embryonic stem cells (ES cells) and induced pluripotent stem cells (iPS cells). A method for differentiating iPS cells into endoderm cells is described in the Examples below. That is, hiPSC clones were differentiated into endoderm cells with a medium consisting of RPMI 1640, 1% B27, 50 ng/mL WNT3A, and 100 ng/mL activin A on a GFR Matrigel- or laminin 511-coated plate for 7 days. At day 0, 10 μM Y-27632 was added and 0.5 mM NaB was added from day 1 to day 3. This method can be modified as appropriate.

The endoderm cells may be those collected from a living body.

The endoderm cells may advantageously be derived from humans, to which they are by no means limited and may be derived from mammals such as mice, rats, guinea pigs, hamsters, rabbits, pigs, cats, dogs, sheep, cows, horses, goats, and monkeys.

By culturing endoderm cells in a medium containing a member of the FGF family, HGF, a member of the IL6 family, and dexamethasone, the endoderm cells can be differentiated into hepatocytes.

As a minimal essential medium, SFD medium (described in WO2016093222), DMEM/F12, DMEM, IMDM, RPMI1640, Williams' Medium E, etc. can be used; a member of the FGF family, HGF, a member of the IL6 family, and dexamethasone can be added to the minimal essential medium.

Concentrations of the member of the FGF family, HGF, the member of the IL6 family, and dexamethasone in the medium can be adjusted as appropriate. For example, when FGF2 is used as the member of the FGF family, the concentration of FGF2 is usually 0.01 to 1000 ng/mL, preferably 0.1 to 100 ng/mL, and more preferably 1 to 50 ng/mL. The concentration of HGF is usually 1 to 1000 ng/mL, preferably 5 to 100 ng/mL, and more preferably 5 to 50 ng/mL. When oncostatin (OSM) is used as the member of the IL6 family, the concentration of OSM is usually 1 to 1000 ng/mL, preferably 5 to 100 ng/mL, and more preferably 10 to 50 ng/mL. The concentration of dexamethasone is usually 1 to 1000000 nM, preferably 10 to 10000 nM, and more preferably 50 to 500 nM.

Nicotinamide may be added to the medium. The concentration of nicotinamide in the medium is usually 0.01 to 1000 mM, preferably 0.1 to 100 mM, and more preferably 1 to 10 mM. Nicotinamide can be added on or after day 8 of culture.

The medium may be a serum-containing medium or a serum-free medium, and in the Examples described below, a serum-free medium was used.

The endoderm cells are preferably seeded on a gel and cultured. The gel to be used is not particularly limited, but GFR Matrigel (produced by Corning Inc.) or the like can be used.

The endoderm cells may be cultured on a container coated with a principal component of tissue basement membrane, such as laminin.

The temperature during culture is not particularly limited but is preferably 30 to 40° C., and more preferably 37° C.

The culture period is not particularly limited but is preferably 1 to 150 days, and more preferably 1 to 90 days.

Differentiation of endoderm cells into hepatocytes can be confirmed by examining the expression of liver-specific genes and hepatic functions or by observing the morphology of hepatocyte-like cells. Examples of hepatocyte-specific genes include TBX3 and TTR, DLK, CK19, EPCAM (hepatoblast), as well as A1AT, ALB, G6PC, ASGR1, TAT, TDO2, CYP2C9, CYP2C19, CYP3A4, and CYP7A1 (hepatocyte). In the process of progression of differentiation of endoderm cells into hepatocytes in the Examples described below, the cells were in the hepatoblast state at the early stage of differentiation (e.g., day 5 of differentiation) and upon further culturing, the cells began to express hepatic functions. The hepatic functions will be described later.

The present invention enables the induction of hepatocytes from pluripotent stem cells in a simple and convenient manner.

Hepatocytes produced by the present method are capable of surviving for a long time, retaining their functionality as hepatocytes, and can realize long-term culture of hepatocytes or mimic the aging process in vitro.

The hepatocytes produced by the method of the present invention can survive in ex vivo monolayer culture for 12 days or more, 15 days or more, 17 days or more, 22 days or more, 26 days or more, 31 days or more, 32 days or more, 42 days or more, 52 days or more, 72 days or more, 82 days or more, 90 days or more, 100 days or more, or even longer while maintaining functionality as hepatocytes. There is no particular upper limit to the period during which the hepatocytes produced by the method of the present invention can survive while maintaining functionality as hepatocytes. The present inventors have observed that the hepatocytes produced by the method of the present invention are viable with the ability to secrete albumin maintained for 82 days.

Examples of the functionality as hepatocytes retained by the hepatocytes produced by the method of the present invention include an albumin secretion ability, drug metabolism capacity, uptake and release of indocyanine green (ICG), glycogen storage, uptake of a low-density lipoprotein, and gene expression. In an embodiment of the present invention, the hepatocytes produced by the method of the present invention are capable of surviving with the ability to secrete albumin maintained for 12 days or more and are also capable of surviving while maintaining functionality as hepatocytes in addition to the albumin secretion ability for 12 days or more.

The albumin secretion ability can be measured by a commercially available albumin ELISA quantification set.

The drug metabolism capacity can be measured by a commercially available Cytochrome P450-related assay kit.

The uptake and release of ICG can be examined by seeding cells in an ICG supplemented medium, incubating them for an appropriate time, and then performing microscopic observation.

Glycogen storage can be examined by detecting glycogen using periodic acid Schiff (PAS) staining.

Low-density lipoprotein uptake can be examined by incubating cells with Dil-Ac-LDL and Hoechst 33342 standard dilute solution and then washing the cells with PBS, followed by image analysis using a fluorescence microscope.

Gene expression tests can be performed as follows. Total RNA is isolated using a PureLink™ RNA Mini Kit. RNA (<2 μg) is used as a template for single-strand cDNA synthesis with a high-capacity cDNA reverse transcription kit according to the manufacturer's instructions. Q-PCR is performed using cDNA as well as specific primers and universal probe library probes.

The hepatocytes produced by the method of the present invention can exhibit aging-related characteristics. Examples of the aging-related characteristics include an increased cell volume, expression of an aging-related gene, an increased inflammatory response, DNA damage, an increased level of intracellular reactive oxygen species, an increased level of cellular senescence-associated β-galactosidase, epigenetic changes, a loss of telomere length, a decline in mitochondrial function, a metabolic disorder, and low responsiveness to growth factors.

The cell volume can be examined by incubating cells with Hoechst 33342 standard dilute solution and then washing the cells with PBS, followed by image analysis using a fluorescence microscope.

Examples of the aging-related gene include CCL2, CDNK2A, CST1, CXCL1, GDF15, ID1, LIMCH1, LMO2, MAP2, MMP24, MYC, RCAN2, S100A8, S100A9, SERPINE1, and TGFB1; expression of the aging-related gene can be measured using SurePrint G3 Human Gene Expression 8×60K (Agilent Technologies, Inc.), which is commercially available.

Examples of the inflammatory response include an increase in inflammation-related factors in cells, and the inflammatory response can be measured using SurePrint G3 Human Gene Expression 8×60K (Agilent Technologies, Inc.), which is commercially available.

The DNA damage can be measured using SurePrint G3 Human Gene Expression 8×60K (Agilent Technologies, Inc.), which is commercially available.

The level of intracellular reactive oxygen species can be measured by adding a reagent such as CellROX Deep Red Reagent (Thermo Fisher Scientific) to the cells and then observing the cells with a microscope or counting the number of fluorescing cells.

The level of cellular senescence-associated β-galactosidase can be measured using a β-galactosidase detection kid (Dojindo).

The epigenetic changes can be measured using Acetyl-Histone H3 Antibody Sampler kit (Cell Signaling Technology, Inc.), which is commercially available.

The telomere length can be measured using a commercially available telomere length qPCR kit (ScienCell Research Laboratories, inc.).

Examples of the mitochondrial function include the production of ATP through oxidative phosphorylation (phosphorylation of ADP) by an electron transport chain, and the decline in mitochondrial function can be confirmed by measuring a decrease in ATP production-related genes using SurePrint G3 Human Gene Expression 8×60K (Agilent Technologies, Inc.), which is commercially available.

Examples of the metabolic disorder include disorders in metabolism as with organic acids, amino acids, uric acid cycle, carbohydrates, fatty acids, lysosoms, lipoproteins, nucleic acids, and membrane carrier proteins, and such metabolic disorders can be confirmed by measuring a change in metabolism-related gene expression using SurePrint G3 Human Gene Expression 8×60K (Agilent Technologies, Inc.), which is commercially available.

The low responsiveness to growth factors can be examined by adding growth factors to cells, culturing the cells for several days, incubating the cells with Hoechst 33342 standard dilute solution and then washing the cells with PBS, followed by analysis of an image captured using a fluorescence microscope.

The hepatocytes produced by the method of the present invention are different in source from hepatocytes derived from a living body. In addition, compared with hepatocytes induced by conventional methods, the hepatocytes produced by the present method are such that the expression of liver-specific genes (ALB, G6PC, ASGR1, TAT, TDO2, CYP2C9, CYP2C19, CYP3A4 and CYP7A1), ALB secretion, as well as the activity of CYP3A4 are greatly upregulated. The hepatocytes induced by conventional methods cannot be cultured in vitro for 30 days or more, whereas the hepatocytes produced by the method of the present invention can be cultured in vitro for 50 days or more and can recapitulate the aging process of hepatocytes.

For the “expression of liver-specific genes, ALB secretion, activity of CYP3A4,” the following tables show some typical numerical values indicating how the hepatocytes produced by the method of the present invention (N-Heps from day 22 of culture in the Examples described below) are upregulated as compared to hepatocytes induced by conventional methods.

Upregulation Ratio for Liver-specific Genes

| | Conventional | Conventional |
|---|---|---|
| | Method A[1] | Method B[2] |
| ALB | 19.82 | 5.88 |
| G6PC | 1,090.51 | 29.23 |
| ASGR1 | 4.75 | 1.02 |
| TAT | 25.65 | 50.64 |
| TDO2 | 17.88 | 5.26 |
| CYP2C9 | 6.43 | 0.84 |
| CYP2C19 | 4.99 | 2.64 |
| CYP3A4 | 5.41 | 8.05 |
| CYP7A1 | 8.34 | 5.33 |

Upregulation Ratio for ALB secretion

| | Conventional | Conventional |
|---|---|---|
| | Method A[1] | Method B[2] |
| ALB Secretion | 4.67 | 2.94 |

Upregulation Ratio for CYP3A4 Activity

| | Conventional | Conventional |
|---|---|---|
| | Method A[1] | Method B[2] |
| CYP3A4 | N.A. | 47.24 |
| Activity | | |

Conventional Method A: Si-Tayeb, K., Noto, F. K., Nagaoka, M., Li, J., Battle, M. A., Duris, C., North, P. E., Dalton, S., and Duncan, S. A. (2010). Highly efficient generation of human hepatocyte-like cells from induced pluripotent stem cells. Hepatology 51, 297-305. Conventional Method B: Kajiwara, M., Aoi, T., Okita, K., Takahashi, R., Inoue, H., Takayama, N., Endo, H., Eto, K., Toguchida, J., Uemoto, S., et al. (2012). Donor-dependent variations in hepatic differentiation from human-induced pluripotent stem cells. Proceedings of the National Academy of Sciences of the United States of America 109, 12538-12543. The cells in the tables were not passaged.

Measurement method for liver-specific genes: Q-PCR (see the Examples below)
Measurement method for ALB secretion: Measurement by ELISA (see the Examples below)
Measurement method for CYP3A4 activity: Measured by P450-Glo™ CYP3A4 Assay Kit (Promega Corporation, V8801), which is commercially available.

When compared to the hepatocytes induced by the conventional method A, the hepatocytes produced by the method of the present invention may be increased in the expression of respective liver-specific genes by the following ratios: ALB by 10 times or more, preferably 15 times or more, more preferably 19 times or more; G6PC by 100 times or more, preferably 500 times or more, more preferably 1000 times or more; ASGR1 by 2 times or more, preferably 3 times or more, more preferably 4 times or more; TAT by 10 times or more, preferably 20 times or more, more preferably 25 times or more; TDO2 by 10 times or more, preferably 15 times or more, more preferably 17 times or more; CYP2C9 by 2 times or more, preferably 4 times or more, more preferably 6 times or more; CYP2C19 by 2 times or more, preferably 3 times or more, more preferably 4 times or more; CYP3A4 by 2 times or more, preferably 4 times or more, more preferably 5 times or more; and CYP7A1 by 3 times or more, preferably 5 times or more, more preferably 8 times or more.

In addition, the secretion of ALB in the hepatocytes produced by the method of the present invention can be increased by 2 times or more, preferably 3 times or more, and more preferably 4 times or more as compared with the hepatocytes induced by the conventional method A.

Further, the hepatocytes produced by the methods of the present invention have CYP3A4 activity, whereas the hepatocytes induced by the conventional method A have no detectable CYP3A4 activity.

When compared to the hepatocytes induced by the conventional method B, the hepatocytes produced by the method of the present invention may be increased in the expression of respective liver-specific genes by the following ratios: ALB by 2 times or more, preferably 4 times or more, more preferably 5 times or more; G6PC by 20 times or more, preferably 25 times or more, more preferably 29 times or more; ASGR1 by a comparable degree; TAT by 30 times or more, preferably 40 times or more, more preferably 50 times or more; TDO2 by 2 times or more, preferably 4 times or more, more preferably 5 times or more; CYP2C9 by a comparable degree; CYP2C19 by 1.2 times or more, preferably 1.5 times or more, more preferably 2 times or more; CYP3A4 by 3 times or more, preferably 5 times or more, more preferably 8 times or more; and CYP7A1 by 2 times or more, preferably 4 times or more, more preferably 5 times or more.

In addition, the secretion of ALB in the hepatocytes produced by the method of the present invention can be increased by 1.2 times or more, preferably 1.5 times or more, and more preferably 2 times or more as compared with the hepatocytes induced by the conventional method B.

Further, the activity of the CYP3A4 in the hepatocytes produced by the method of the present invention can be increased by 30 times or more, preferably 40 times or more, and more preferably 47 times or more as compared with the hepatocytes induced by the conventional method B.

The following tables show some typical measurements of the liver-specific gene expression, ALB secretion, and CYP3A4 activity for the hepatocytes produced by the method of the present invention (N-Heps from day 22 of culture in the Examples described below), the hepatocytes induced by the conventional method A and the hepatocytes induced by the conventional method B.

Q-PCR Measurement

| | | Conventional | Conventional |
|---|---|---|---|
| | N-Hep | Method A[1] | Method B[2] |
| ALB | | | |
| | 13.87 | 0.6162 | 2.425 |
| | 14.68 | 0.6988 | 2.167 |
| | 12.48 | 0.7732 | 1.959 |
| | 9.346 | 0.4534 | 2.018 |
| G6PC | | | |
| | 0.1671 | 0.000141 | 0.00517 |
| | 0.1547 | 0.000154 | 0.00555 |
| | 0.1522 | 0.000198 | 0.00506 |
| | 0.1411 | 0.0000711 | 0.00527 |
| ASGR1 | | | |
| | 0.1631 | 0.0356 | 0.1734 |
| | 0.1658 | 0.0349 | 0.1573 |
| | 0.1693 | 0.039 | 0.147 |
| | 0.1515 | 0.0272 | 0.165 |
| TAT | | | |
| | 0.0000884 | 0.00000151 | 0.00000559 |
| | 0.000123 | 0.00000248 | 0.00000126 |
| | 0.000136 | 0.000011 | 0.00000068 |
| | 0.000121 | 0.00000327 | 0.00000172 |
| TDO2 | | | |
| | 0.1723 | 0.00874 | 0.0281 |
| | 0.1527 | 0.00798 | 0.0252 |
| | 0.1321 | 0.00991 | 0.0346 |
| | 0.1396 | 0.00675 | 0.0255 |
| CYP2C9 | | | |
| | 0.00357 | 0.00062 | 0.00372 |
| | 0.0036 | 0.000454 | 0.00406 |
| | 0.00277 | 0.000511 | 0.00359 |
| | 0.00275 | 0.000389 | 0.00389 |
| CYP2C19 | | | |
| | 0.000861 | 0.000131 | 0.000315 |
| | 0.00133 | 0.000197 | 0.000399 |
| | 0.000735 | 0.000253 | 0.000305 |
| | 0.000659 | 0.000138 | 0.000346 |
| CYP3A4 | | | |
| | 0.0000954 | 0.0000168 | 0.00000572 |
| | 0.0000627 | 0.0000139 | 0.0000163 |
| | 0.000103 | 0.0000147 | 0.000015 |
| | 0.0000983 | 0.000021 | 0.00000764 |
| CYP7A1 | | | |
| | 0.000158 | 0.0000088 | 0.000023 |
| | 0.000108 | 0.0000103 | 0.000027 |
| | 0.0000754 | 0.0000238 | 0.000021 |
| | 0.000163 | 0.0000176 | 0.000019 |

ALB Secretion (μg/ml/24 h/1 million)

| | Conventional | Conventional | Human |
|---|---|---|---|
| N-Hep | Method A[1] | Method B[2] | Hepatocyte |
| 7.64 | 2.76 | 1.66 | 5.54 |
| 8.44 | 2.41 | 1.72 | 5.16 |
| 6.63 | 2.24 | 1.82 | 5.53 |
| 5.30 | 2.42 | 1.36 | 4.13 |
| 8.01 | 2.97 | | 5.47 |
| 9.16 | 2.60 | | 5.71 |
| 7.52 | 2.39 | | |
| 7.72 | 2.56 | | |
| 7.64 | 3.03 | | |
| 8.44 | | | |

CYP3A4 Activity (RLU/4 h/ml/million cells)

| | Conventional | Human |
|---|---|---|
| N-Hep | Method B | Hepatocyte |
| 255647.3 | 4922.5 | 191833.3 |
| 242359.6 | 5372.5 | 199833.3 |
| 222521.7 | 4792.5 | 201166.7 |
| 238055.1 | 5202.5 | 185500 |
| 303116.1 | | |
| 307353 | | |
| 320265.5 | | |
| 299080.9 | | |

N-Hep: Hepatocytes produced by the method of the present invention (N-Hep at day 22 of culture in Examples described below)
Human hepatocytes: IVT-F00995-P-AKB from BioreclamationIVT (a female donor at age 39 years)
The cells in the tables were not passaged.
Measurement method for liver-specific genes: Q-PCR (see the Examples below)
Measurement method for ALB secretion: Measurement by ELISA (see the Examples below)
Measurement method for CYP3A4 activity: Measurement by P450-Glo™ CYP3A4 Assay Kit (Promega Corporation, V8801), which is commercially available.

The hepatocytes produced by the method of the present invention have the following expression levels measured by Q-PCR: 3 or more, preferably 5 or more, more preferably 9 or more for ALB; 0.05 or more, preferably 0.1 or more, more preferably 0.14 or more for G6PC; 0.05 or more, preferably 0.1 or more, more preferably 0.16 or more for ASGR1; 0.00001 or more, preferably 0.00005 or more, more preferably 0.0001 or more for TAT; 0.01 or more, preferably 0.05 or more, more preferably 0.1 or more for TDO2; 0.05 or more, preferably 0.01 or more, more preferably 0.02 or more for CYP2C9; 0.00005 or more, preferably 0.0001 or more, more preferably 0.0006 or more for CYP2C19; 0.00001 or more, preferably 0.00005 or more, more preferably 0.00009 for CYP3A4; and 0.00001 or more, preferably 0.00005 or more, more preferably 0.00007 or more for CYP7A1.

The hepatocytes produced by the method of the present invention can have an ALB secretion of 2 μg/ml/24h/1 million or more, preferably 4 μg/ml/24h/1 million or more, and more preferably 5 μg/ml/24h/1 million or more, as quantified by ELISA.

The hepatocytes produced by the method of the present invention can have a CYP3A4 activity of 50000 RLU/4h/ml/million cells or more, preferably 100000 RLU/4h/ml/million cells or more, and more preferably 200000 RLU/4h/ml/million cells or more, as measured by the commercially available P450-Glo™ CYP3A4 Assay Kit (Promega Corporation, V8801).

2. Induction of Hepatic Progenitor Cells and Induction of Differentiation of Hepatic Progenitor Cells into Hepatocytes or Cholangiocytes

The present invention also provides a method for producing hepatic progenitor cells, the method including culturing hepatocytes in the presence of a member of the FGF family.

In the present invention, examples of the member of the FGF family include FGF2, FGF1, FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF22, and FGF23, with FGF2 being preferred.

Any hepatocytes that express liver-specific genes and exhibit hepatic function can be used for inducing hepatic progenitor cells, pHC, or plasticity; the hepatocytes may be any of hepatocytes differentiated from pluripotent stem cells, cells obtained by passaging hepatocytes differentiated from pluripotent stem cells, primary culture hepatocytes isolated from a biological tissue, cells obtained by passaging primary culture hepatocytes isolated from a biological tissue, or combinations thereof.

Examples of the pluripotent stem cells include ES cells and iPS cells.

Examples of the hepatocytes into which pluripotent stem cells differentiate include the hepatocytes described in 1 above, i.e., hepatocytes into which endoderm cells are induced to differentiate in the presence of a member of the FGF family, HGF, a member of the IL6 family, and dexamethasone. In the Examples below, endoderm cells were seeded on a GFR Matrigel- or laminin 511-coated plate using a hepatic differentiation medium (HDM) without nicotinamide; from day 8 to day 10, the medium was daily replaced with HDM; from day 11, the medium was replaced every 2 days, whereby the endoderm cells were induced to differentiate into hepatocytes. The HDM consisted of SFD containing 10 ng/mL FGF2, 20 ng/mL HGF, 10 ng/mL OSM, 100 nM dexamethasone, and 10 mM nicotinamide. This method can be modified as appropriate. For their passage, these hepatocytes may be seeded on a GFR Matrigel- or laminin 511-coated plate using a nicotinamide-containing HDM, with the medium being replaced every 2 days to passage the hepatocytes. The HDM consisted of SFD containing 10 ng/mL FGF2, 20 ng/mL HGF, 10 ng/mL OSM, 100 nM dexamethasone, and 10 mM nicotinamide. This method can be modified as appropriate.

When hepatocytes differentiated from pluripotent stem cells are used to induce hepatic progenitor cells, the number of passages of the hepatocytes is usually 1 to 5, preferably 1 to 4, more preferably 1 to 3, and the number of days for culturing the hepatocytes is usually 1 to 100 days, preferably 1 to 80 days, more preferably 5 to 60 days.

The hepatocytes may be primary hepatocytes isolated from biological tissue, or cells obtained by passaging the isolated primary hepatocytes. In the Examples below, primary human hepatocytes from donors at age 2 months, 39 years, and 78 years were used. For their culture or passage, these primary human hepatocytes may be seeded on a GFR Matrigel- or laminin 511-coated plate using a human hepatocyte culture medium, with the medium being replaced every 2 days to culture or passage the primary human hepatocytes. There are two types of human hepatocyte culture media. One was HDM, which consisted of SFD containing 10 ng/mL FGF2, 20 ng/mL HGF, 10 ng/mL OSM, 100 nM dexamethasone, and 10 mM nicotinamide. The other was Williams E medium containing 5% FBS, 1 μM dexamethasone, 4 μg/mL Human Recombinant Insulin, 2 mM GlutaMAX, and 15 mM HEPES. This method can be modified as appropriate.

When hepatic progenitor cells are induced from primary culture cells isolated from human biological tissue, cells obtained by passaging these primary culture cells, or a combination thereof, the age of the human is usually 0 to 120 years, preferably 0 to 100 years, and more preferably 0 to 80 years. When primary culture cells isolated from biological tissue of an animal other than humans, cells obtained by passaging these primary culture cells, or a combination thereof are used, an appropriate weekly, monthly, or yearly age can be estimated from the human case. The number of passages of the culture cells is usually 1 to 5, preferably 1 to 4, more preferably 1 to 3, and the number of days for culturing the cells is usually 1 to 30 days, preferably 1 to 20 days, more preferably 1 to 10 days.

The hepatocytes may be a combination of hepatocytes differentiated from pluripotent stem cells, cells obtained by passaging the hepatocytes differentiated from pluripotent stem cells, primary culture hepatocytes isolated from a biological tissue, and cells obtained by passaging the primary culture hepatocytes isolated from a biological tissue.

The hepatocytes may be derived from humans to which they are by no means limited and may be derived from mammals such as mice, rats, guinea pigs, hamsters, rabbits, pigs, cats, dogs, sheep, cows, horses, goats, and monkeys.

The hepatic progenitor cells can be induced (hereinafter sometimes referred to as “reprogrammed”) by culturing the hepatocytes in a medium containing a member of the FGF family.

As a minimal essential medium, SFD medium (described in WO2016093222), DMEM/F12, DMEM, IMDM, RPMI1640, Williams' Medium E, etc. can be used; a member of the FGF family can be added to the minimal essential medium.

A concentration of the member of the FGF family in the medium can be adjusted as appropriate. For example, when FGF2 is used as the member of the FGF family, the concentration of FGF2 is usually 0.01 to 1000 ng/mL, preferably 0.1 to 100 ng/mL, and more preferably 1 to 50 ng/mL.

EGF, HGF, a ROCK inhibitor, a selective inhibitor of ALK4, ALK5 or ALK7 (a TGF-β receptor inhibitor), or a GSK-313 inhibitor may be added to the medium.

Epidermal growth factor (EGF), which is a 6045-Da protein that has 53 amino acid residues and three intramolecular disulfide bonds, binds as a ligand to the epidermal growth factor receptor (EGFR) on the cell surface and plays an important role in the regulation of cell growth and proliferation. The concentration of EGF in the medium is usually 0.1 to 1000 ng/mL, preferably 1 to 100 ng/mL, and more preferably 1 to 50 ng/mL.

HGF has been described above. The concentration of HGF is usually 1 to 1000 ng/mL, preferably 5 to 100 ng/mL, and more preferably 5 to 50 ng/mL.

Rho-kinase (Rho-associated protein kinase: ROCK) is a serine-threonine protein phosphorylase identified as a target protein of small molecular weight GTP-binding protein Rho; examples of the ROCK inhibitor include Y-27632, AT13148, Fasudil, Hydroxyfasudil, GSK269962A, GSK180736A, GSK429286A, KD025, Netarsudil, RKI-1447, Thiazovivin, and Y-39983. When Y-27632 is used as the ROCK inhibitor, the concentration of Y-27632 is usually 1 to 1000 μM, preferably 1 to 100 μM, and more preferably 1 to 50 μM.

ALK4, ALK5, and ALK7 are TGF-β1 activin receptor-like kinases (ALKs); examples of the selective inhibitor of ALK4, ALK5 or ALK7 (TGF-β receptor inhibitor) include A83-01, DMH1, GW788388, Galunisertib, K02288, LDN-193189, LDN-212854, LDN-214117, LY2109761, LY364947, ML347, SB431542, SB505124, SB525334, SD-208, RepSox, and Vactosertib. When A83-01 is used as the selective inhibitor of ALK4, ALK5 or ALK7 (TGF-β inhibitor), the concentration of A83-01 is usually 0.01 to 100 μM, preferably 0.1 to 10 μM, and more preferably 0.1 to 5 μM.

GSK3β is a serine-threonine protein kinase; examples of the GSK-313 inhibitor include CHIR99021, 1-Azakenpaullone, 2-D08, AR-A014418, AZD1080, AZD2858, Bikinin, BIO, BIO-acetoxime, CHIR-98014, IM-12, Indirubin, LY2090314, SB216763, SB415286, Tideglusib, TDZD-8, and TWS119. When CHIR99021 is used as the GSK-3β inhibitor, the concentration of CHIR99021 is usually 0.01 to 100 μM, preferably 0.01 to 20 μM, and more preferably 0.1 to 20 μM.

The medium may be a serum-containing medium or a serum-free medium, and in the Examples described below, a serum-free medium was used.

The hepatocytes are preferably seeded on a gel and cultured. The gel to be used is not particularly limited, and GFR Matrigel (produced by Corning Inc.) or the like can be used.

The hepatocytes may be cultured on a container coated with a principal component of tissue basement membrane, such as laminin.

The temperature during culture is not particularly limited but is preferably 30 to 40° C., and more preferably 37° C.

The culture period is not particularly limited but is preferably 4 to 15 days, and more preferably 5 to 10 days.

The hepatic progenitor cells induced from hepatocytes by the method of the present invention are proliferative and have a differentiation ability as exemplified by alpha-fetoprotein (AFP)-negative hepatic progenitor cells that have proliferative ability and a bidirectional differentiation ability into hepatocytes and bile duct epithelial cells.

The hepatic progenitor cells of the present invention feature high expression of cell cycle genes (positive), high expression of hepatic-stem/progenitor cell-related genes (positive), low expression of hepatic-function-related genes (negative), manifestation of positive staining for HNF4A, SOX9, and CK19 but not for ALB and AFP, etc. (see the Examples below).

The hepatic progenitor cells can be passaged by seeding the cells onto a Matrigel or laminin coating using a medium including a member of the FGF family and then replacing the medium with a ROCK-inhibitor-free medium at an appropriate time.

The proliferative status of hepatic progenitor cells can be examined by calculating a cell population doubling time, as described in the Examples below. The hepatic progenitor cells can have a high proliferative ability as evidenced by a cell population doubling time of 14 to 36 hours.

The hepatic progenitor cells as induced from hepatocytes by the method of the present invention can differentiate into hepatocytes, cholangiocytes, etc.

The hepatic progenitor cells may be differentiated into hepatocytes by culturing in s hepatic differentiation medium. As the hepatic differentiation medium, HDM (described in 1 above) can be used. A vitamin A metabolite (e.g., retinoic acid (RA)) may be added to the hepatic differentiation medium at an appropriate time and for an appropriate period. In the Examples described below, the medium was replaced with a RA-containing HDM for 8 days and then with a RA-free HDM for another 7 days. The medium was replaced every two days. The total hepatic differentiation period was 15 days. This method can be modified as appropriate. Hepatocytes differentiated from hepatic progenitor cells can show enhanced expression of functional genes (e.g., AAT, CTP2C9, CYP2C19, etc.) and enhanced ALB secretion. Moreover, hepatocytes differentiated from hepatic progenitor cells can form tight junctions between themselves to acquire ammonia clearance capacity and hepatic functions (ICG uptake and release, glycogen storage, and low-density lipoprotein uptake).

To differentiate hepatic progenitor cells into cholangiocytes, the hepatic progenitor cells may be cultured in a cholangiocyte differentiation medium (CDM) (see Francis et al., 2004; Sampaziotis et al., 2015; Sampaziotis et al., 2017). In the Example described below, hepatic progenitor cells mixed with GFR-Matrigel were seeded on a non-treated 24-well plate as a mount drop and incubated at 37° C. for 30 min in a 5% CO₂ incubator. Then, a cholangiocyte differentiation medium (CDM) was filled into the wells. The CDM consisted of SFD containing 10 ng/mL EGF, 20 ng/mL HGF, 50 ng/mL WNT3A, 100 ng/mL R-spondin-1, 50 ng/mL FGF10, 3 μM RA, 10 μM Y-27632 and 10 μM forskolin. The medium was replaced every three days. This method can be modified as appropriate.

Cholangiocytes differentiated from the hepatic progenitor cells can grow into a ring-like structure and then gradually develop into a cyst structure. According to Q-PCR analysis, the cyst structure can feature upregulated transcription of cholangiocyte signature genes (FIGS. 3-1 G and 3-2 H). The cholangiocytes of these cysts can have positive results of staining of CK19, F-ACTIN, and SOX9 with negative staining of AFP, ALB, and HNF4A. Moreover, these cysts can possess mature bile duct features (including a lumen with an apical and basolateral plasma membrane, microvilli, primary cilia on the apical membrane, tight junctions, and multivesicular bodies). These cholangiocytes can have bile duct functions of 1) transporting Rhodamine 123 into the lumen and 2) specifically exporting bile acid from the lumen.

Thus, the present invention provides a method for producing hepatocytes, the method including inducing differentiation of hepatic progenitor cells into hepatocytes, the hepatic progenitor cells being obtained by the method for producing hepatic progenitor cells that includes culturing hepatocytes in the presence of a member of the FGF family. The present invention also provides a method for producing cholangiocytes, the method including inducing differentiation of hepatic progenitor cells into cholangiocytes, the hepatic progenitor cells being obtained by the method for producing hepatic progenitor cells that includes culturing hepatocytes in the presence of a member of the FGF family. The present invention also provides a medium containing FGF10, retinoic acid, and forskolin for inducing differentiation of the hepatic progenitor cells into cholangiocytes. The medium of the present invention may include other components that are included in the cholangiocyte differentiation medium (CDM) described above. Further, the present invention provides a kit for a culture medium in induced differentiation, the kit including: an agent containing FGF10, retinoic acid, and forskolin for use in a culture medium to be used to induce differentiation of the hepatic progenitor cells into cholangiocytes; and instructions of the use of the agent in the culture medium to be used to induce the differentiation. The agent containing FGF10, retinoic acid, and forskolin may also include other components included in the above cholangiocyte differentiation medium (CDM).

The hepatic progenitor cells induced from hepatocytes by the method of the present invention are capable of proliferating for a long time and can be passaged, say, at least 20 times or more. Thus, the present invention enables the mass production of hepatocytes or cholangiocytes.

### 3. Improvement of the Plasticity of Aging Hepatocytes

In 2 above, the medium including a member of the FGF family may be further supplemented with a drug causing histone hyperacetylation, whereupon the plasticity of old hepatocytes can be improved to induce hepatic progenitor cells.

The present invention provides a method for producing hepatic progenitor cells, the method including culturing hepatocytes in the presence of a member of the FGF family and a drug causing histone hyperacetylation.

By means of the drug causing histone hyperacetylation, the aging of hepatocytes can be suppressed to induce their plasticity. As used herein, the term “hepatocyte plasticity” refers to the ability of hepatocytes to convert into proliferative hepatic progenitor cells having an ability to differentiate into both hepatocytes and cholangiocytes. Improved hepatocyte plasticity leads to an increase in the responsiveness of the hepatocytes to the induction of their proliferation.

Thus, the present invention provides a method for suppressing hepatocyte aging by using a drug causing histone hyperacetylation. In another aspect, the present invention provides an agent for suppressing the aging of hepatocytes (anti-aging drug) including a drug causing histone hyperacetylation as an active ingredient. The anti-aging drug of the present invention can be applied both in vitro (cultured cells) and in vivo (cells within a living body).

Hepatocyte aging can be confirmed by decreased cell proliferation, diminished functionality as hepatocytes, upregulation of an aging-related gene, expression of an aging-related marker, or the like. Hepatocyte aging can also be confirmed by an increased cell volume, an increased inflammatory response, DNA damage, epigenetic changes, a loss of telomere length, a decline in mitochondrial function, a metabolic disorder, low responsiveness to growth factors, or the like.

The cell proliferation can be examined by calculating a cell population doubling time.

The functionality as hepatocytes and the measurement method therefor have been described in 1 above.

The function of the aging-related gene and the measurement method therefor have also been described in 1 above.

Examples of the aging-related marker include a level of intracellular reactive oxygen species, and a level of cellular senescence-associated β-galactosidase; the measurement methods therefor have been described in 1 above.

The measurement methods for an increased cell volume, an increased inflammatory response, DNA damage, epigenetic changes, a loss of telomere length, a decline in mitochondrial function, a metabolic disorder, and low responsiveness to growth factors have also been described in 1 above.

The suppression of hepatocyte aging can be confirmed by measuring the above markers or events.

The present invention also provides a method for increasing hepatocyte plasticity using a drug causing histone hyperacetylation. In another aspect, the present invention provides a drug that increases hepatocyte plasticity and includes a drug causing histone hyperacetylation as an active ingredient. The increased plasticity allows the induction of hepatic progenitor cells even from old hepatocytes derived from pluripotent stem cells or from hepatocytes derived from the elderly hepatocytes. The increased plasticity also allows enhanced responsiveness of the hepatocytes to the induction of their proliferation. The drug of the present invention can be a regeneration-inducing agent that improves the regenerative capacity of old cells. The drug of the present invention can promote liver regeneration in the elderly. The drug of the present invention also allows for successful transplantation therapy using grafts or cells from elderly donors. The drug of the present invention can be applied both in vitro (culture cells) and in vivo (cells within a living body).

The drug causing histone hyperacetylation may be used in combination with a member of the FGF family. Examples of the member of the FGF family include FGF2, FGF1, FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF22, FGF23, or combinations thereof, with FGF2 being preferred.

Examples of the drug causing histone hyperacetylation include a histone deacetylase inhibitor; examples of the histone deacetylase inhibitor include Trichostatin A, Tacedinaline (CI-994), M344, ITSA-1, Sodium valproate, Sodium 4-phenylbutuyrate, Sodium Butyrate (NaB), valproic acid (VPA), Abexinostat (PCI-24781), Belinostat (PXD101), Citarinostat (ACY-241), Dacinostat (LAQ824), Depudecin, Domatinostat (4SC-202), Droxinostat, Entinostat (MS-275, SNDX-275), Fimepinostat (CUDC-907), Givinostat (ITF2357), Mocetinostat (MGCD0103), Nexturastat A, Panobinostat (LBH-589,NVP-LBH589), Pracinostat (SB939), Quisinostat (JNJ-26481585) 2HC1, Resminostat, Ricolinostat (ACY-1215), Tucidinostat (Chidamide), Vorinostat (SAHA), ACY-738, Apicidin, AR-42, BG45, BML-210, BRD73954, CAY10603, CUDC-101, Curcumin, Depudecin, H1388, HC Toxin, HPOB, LMK-235, MC1568, Oxamflatin, (−)-Parthenolide, PCI-34051, RG2833 (RGFP109), RGFP966, Romidepsin (FK228, Depsipeptide), Santacruzamate A (CAY10683), Scriptaid, SKLB-23bb, Splitomicin, Suberoyl bis-hydroxamic acid, Tasquinimod, TH34, Tinostamustine (EDO-S101), TMP195, TMP269, Tubacin, Tubastatin A, and combinations thereof.

Hepatocytes whose aging is to be suppressed or whose plasticity is to be increased through the drug causing histone hyperacetylation are preferably old hepatocytes, to which they are by no means limited. The hepatocytes may be any of the hepatocytes that have been differentiated from pluripotent stem cells, cells obtained by passaging hepatocytes differentiated from pluripotent stem cells, primary culture hepatocytes isolated from a biological tissue, cells obtained by passaging primary culture hepatocytes isolated from a biological tissue, or combinations thereof.

When the hepatocytes are hepatocytes differentiated from pluripotent stem cells, the “old hepatocytes” refer to, for example, hepatocytes that have undergone passage for 50 days or more including all steps after they are induced to differentiate from pluripotent stem cells (e.g., iPSCs) (after 22 days).

When the hepatocytes are cells obtained by passaging hepatocytes differentiated from pluripotent stem cells, the “old hepatocytes” refer to, for example, cells obtained by passaging hepatocytes once or more which have undergone passage for 25 days or more from pluripotent stem cells (e.g., iPSCs).

When the hepatocytes are primary culture cells isolated from liver tissue, the “old hepatocytes” refer, in the case of humans, for example, to hepatocytes isolated from tissues of humans at age 65 years or older.

When the hepatocytes are cells obtained by passaging primary culture hepatocytes isolated from a biological tissue, the “old hepatocytes” refer, in the case of humans, for example, to hepatocytes cultured for 20 days or more after the isolation.

The hepatocytes may be derived from humans, to which they are by no means limited, and they may be derived from mammals such as mice, rats, guinea pigs, hamsters, rabbits, pigs, cats, dogs, sheep, cows, horses, goats, and monkeys.

Culturing hepatocytes in a medium including the drug causing histone hyperacetylation can suppress their aging and increase their plasticity.

As the medium, the medium described in 2 above (medium used for induction (reprogramming) from hepatocytes into hepatic progenitor cells) may be used. That is, as a minimal essential medium, SFD medium (described in WO2016093222), (DMEM/F12, DMEM, IMDM, RPMI1640, Williams' Medium E), etc. can be used; a member of the FGF family can be added to the minimal essential medium.

A concentration of the member of the FGF family in the medium can be adjusted as appropriate. For example, when FGF2 is used as the member of the FGF family, the concentration of FGF2 is usually 0.01 to 1000 ng/mL, preferably 0.1 to 100 ng/mL, and more preferably 1 to 50 ng/mL.

Another component that may be added to the medium and the concentration thereof are also described in 2 above.

The medium may be a serum-containing medium or a serum-free medium, and in the Examples described below, a serum-free medium was used.

The hepatocytes are preferably seeded on a gel and cultured. The gel to be used is not particularly limited, but GFR Matrigel (produced by Corning Inc.) or the like can be used.

The hepatocytes may be cultured on a container coated with a principal component of a tissue basement membrane, such as laminin.

The temperature during culture is not particularly limited but is preferably 30 to 40° C., and more preferably 37° C.

The culture period is not particularly limited but is preferably 4 to 15 days, and more preferably 5 to 10 days.

### 4. Application

The present invention enables the mass production of hepatocytes and cholangiocytes from pluripotent stem cells (e.g., iPS cells).

Human hepatocytes, etc. created by the method of the present invention can be used for industrial applications such as in vitro drug metabolism tests, hepatotoxicity tests, and hepatitis virus infection tests.

Human hepatocytes, etc. created by the method of the present invention include the following:

1. Hepatocytes produced by a method for producing hepatocytes, the method comprising differentiating endoderm cells into hepatocytes in the presence of a member of the FGF family, HGF, a member of the IL6 family, and dexamethasone;
2. Alpha-fetoprotein (AFP)-negative hepatic progenitor cells produced using a method for producing hepatic progenitor cells that includes culturing hepatocytes in the presence of a member of the FGF family, the AFP-negative hepatic progenitor cells having proliferative ability and a bidirectional differentiation ability into hepatocytes and bile duct epithelial cells;
3. Cells (e.g., hepatocytes, cholangiocytes) generated by induced differentiation from the hepatic progenitor cells of 2.

The human hepatocytes, etc. created by the method of the present invention can be applied to regenerative medicine as the main component of a composition for transplantation. The transplant site for hepatocytes may be any site where the hepatocytes can be transplanted, and examples thereof include intracranium, mesentery, liver, spleen, kidney, subrenal capsule, and portal vein. The number of hepatocytes per transplantation may be 100000 to 100000000, preferably 1000000 to 50000000, and more preferably 1000000 to 10000000 per cm² of a transplant site. For the transplantation, EGF, HGF, ROCK inhibitors, TGF-β receptor inhibitors, GSK-3β inhibitors, etc. can be used. The composition for transplantation may include a drug that increases hepatocyte plasticity (described above).

In addition, the human hepatocyte, etc. created by the method of the present invention can be used to produce an artificial liver.

Furthermore, the human hepatocyte, etc. created by the method of the present invention can be transplanted into a non-human animal to produce a chimeric animal. A non-human animal (e.g., mice) transplanted with cells can mimic the physiological function of the biological species (e.g., human) from which the transplanted cells are derived. This animal can be used to conduct drug metabolism tests and safety tests on seed compounds for drug discovery. The non-human animal may advantageously have liver failure. Liver failure can be induced by administration of ganciclovir. When hepatic progenitor cells are transplanted into a non-human animal, the transplanted hepatic progenitor cells can proliferate and/or differentiate within the non-human animal. Transplantation of the hepatocytes produced by the method of the present invention promotes liver regeneration in a non-human animal. Examples of the non-human animal include mice and rats. The site for transplantation and the number of hepatocytes used for the transplantation may be the same as in the case of transplantation into humans.

Furthermore, the present invention makes it possible to promote liver regeneration by the use of the drug causing histone hyperacetylation. The present invention provides a liver regeneration accelerator that includes a drug causing histone hyperacetylation as an active ingredient. The present invention provides a method for promoting liver regeneration, the method comprising administering to a subject a pharmaceutically effective amount of a drug causing histone hyperacetylation. The drug causing histone hyperacetylation has been described above.

The drug causing histone hyperacetylation can promote regeneration of a liver that has suffered a liver disease. The liver disease can be caused by viral hepatitis (due to infection with Hepatitis virus A, Hepatitis virus B, Hepatitis virus C, Hepatitis virus D, Hepatitis virus E), alcoholic hepatitis, autoimmune hepatitis, primary cirrhotic liver, drug-induced hepatitis, fatty liver (alcoholic, non-alcoholic), surgical removal of the liver, a liver injury due to a trauma, aging, hepatic fibrosis, a liver disease associated with obesity, or the like. The drug causing histone hyperacetylation can be used as a medicine to treat and/or prevent these diseases or disorders.

The drug causing histone hyperacetylation may be in the form of a salt or solvate.

The drug causing histone hyperacetylation can be administered to a subject (human or animal) as a pharmaceutical preparation (e.g., an injection, a capsule, a tablet, powder, granules, etc.) that has been formulated according to the conventional method. For example, when the drug causing histone hyperacetylation is NaB, it may advantageously be administered orally or parenterally (e.g., nasally, rectally, transdermally, subcutaneously, intravenously, intramuscularly, etc.) at a dosage of about 10 to 100000 mg/kg (body weight) per day, preferably about 100 to 10000 mg/kg (body weight) per day in terms of the amount of the active ingredient in one or several divided doses; however, the dosage or the number of doses can be appropriately changed according to symptoms, age, administration methods, or the like. In the case of using drugs other than NaB that cause histone hyperacetylation, they may advantageously be used at a dosage that delivers an effect comparable to the above dosage of NaB. For formulation into an injection, a carrier such as distilled water or physiological saline may advantageously be used, and for formulation into a capsule, a tablet, powder, or granules, an excipient such as starch, lactose, sucrose, or calcium carbonate; a binder such as starch paste, gum arabic, gelatin, sodium alginate, carboxymethyl cellulose, or hydroxypropyl cellulose; a lubricant such as magnesium stearate or talc; and a disintegrator such as starch, agar, microcrystalline cellulose, calcium carbonate, sodium hydrogen carbonate, or sodium alginate may be used. The content of the active ingredient in the preparation can vary between 1 and 99% by weight. For example, in the case of taking the form of a tablet, a capsule, granules, powder, or the like, the content of the active ingredient is preferably 5 to 80% by weight, and in the case of an injection, the content of the active ingredient is preferably 1 to 10% by weight.

### EXAMPLES

Hereinbelow, the present invention will be described in detail with reference to the following Examples.

### Example 1

Histone Hypoacetylation Impairs the Plasticity of Human Hepatocytes with Aging

Our results should make liver transplants from the elderly more useful in the clinic; normally, the hepatic tissues and hepatocytes of the elderly have low regenerative capacity. We found how to easily obtain hepatocytes from human induced pluripotent stem cells. These hepatocytes recapitulated hepatic aging in a culture dish, and we found that hepatic plasticity (responsiveness to proliferation induction) is impaired by aging-associated histone hypoacetylation. Promotion of histone acetylation improved the plasticity of old hepatocytes and enhanced their regenerative capacity in a liver injury model.

### SUMMARY

Hepatocyte plasticity plays a critical role in liver regeneration and decreases with aging, but the fundamental principles underlying this decrease are unclear. Here, in order to elucidate the underlying mechanism, we devised a research strategy involving hepatocytes derived from human induced pluripotent stem cells (hiPSC-Heps) and accumulated during prolonged sub-culture of hiPSC-Heps the changes in aging-related hallmarks and the information on the expression of an aging-related set of genes. Moreover, we identified that the plasticity of human hepatocytes was regulated by the FGF2-MAPK-EZH2 axis and gradually diminished with aging. Notably, the impaired plasticity of aging hepatocytes strongly correlated with histone hypoacetylation. Selective inhibition of histone deacetylases markedly improved the plasticity of old hiPSC-Heps and primary human hepatocytes, and this effect increased the repopulation ability of old primary human hepatocytes in a liver injury model. Thus, aging-associated histone hypoacetylation impairs hepatocyte plasticity, and histone acetylation can be a therapeutic target for improvement of liver regenerative capacity in the elderly.

### INTRODUCTION

Liver regeneration is a unique phenomenon allowing the liver to regenerate a new tissue to replace lost parts (dead cell mass and tissue); due to this phenomenon, the liver seems to be a non-aging organ (Timchenko, 2009). Nonetheless, clinical reports indicate that aging is a major risk factor for liver diseases including nonalcoholic fatty liver disease, alcoholic liver disease and hepatitis C (Kim et al., 2015), and liver transplantation from the elderly yields worse survival and worse overall outcomes than transplantation from young donors (Durand et al., 2019; Germani et al., 2012). These indicate that aging may compromise the liver regenerative capacity. Actually, the aging-associated decline of liver regeneration was discovered half a century ago when researchers noticed a considerably reduced proliferative response in an old rat liver after partial hepatectomy (Bucher et al., 1964). This phenomenon has been confirmed in other rodent models (Fry et al., 1984; Iakova et al., 2003), and several aging-associated alterations that may be linked to the impairment of regeneration (e.g., a metabolic abnormality, epigenetic changes, low responsiveness to growth factors, and a loss of telomere length) have been found (Aikata et al., 2000; Sato et al., 2017; Sawada, 1989; Timchenko, 2009). Yet, how aging contributes to the impairment of liver regeneration and how to improve this regenerative capacity in the elderly are still unknown, especially in humans.

Hepatocytes constitute over 70% of liver mass, and accumulated evidence suggests that hepatocyte plasticity plays a critical part in the maintenance of regenerative capacity of the liver (Kopp et al., Li et al., 2016). Lineage-tracing experiments in mice have shown that in response to liver injury, hepatocytes can convert into proliferative progenitor-like cells to replace the lost hepatocytes and cholangiocytes (Tarlow et al., 2014; Yanger et al., 2014; Yanger et al., 2013). Such progenitor-like cells were also detected in a cirrhotic human liver (Deng et al., 2018). Therefore, studies on the intrinsic connections between aging and hepatocyte plasticity should help to understand the role of aging in human liver regeneration. Due to the limitations of tracing experiments on human beings, we wondered whether the plasticity of human hepatocytes can be modeled in a culture dish. Recently, several groups reported that primary human hepatocytes (PHHs) can be induced into a proliferative state with a repopulation ability (Fu et al., 2019; Kim et al., 2019; Zhang et al., 2018), but these proliferative PHHs have only a limited bipotential differentiation ability. What is more, currently available PHHs are derived only from liver tissue of cadavers or patients having liver diseases with complex and unreliable extrinsic factors. Such extrinsic factors make the above-mentioned PHHs difficult to use for the discovery of valuable connections between aging and hepatocyte plasticity.

To unravel these intrinsic connections, the ideal experimental groups of hepatocytes must have the same origin, i.e., must be sampled from the same person at different ages, but it is difficult to obtain such hepatocytes from the same donor. In the past decade, there has been extensive progress in the directed differentiation of human induced pluripotent stem cells (hiPSCs) for application to regenerative medicine and disease modeling (Stadtfeld and Hochedlinger, 2010; Studer et al., 2015), and some laboratories have adopted a method that enables the use of hiPSCs for modeling aging-associated neurodegenerative diseases (Miller et al., 2013; Vera et al., 2016). Unfortunately, no hiPSC-derived hepatocytes (hiPSC-Heps) that can effectively recapitulate the biological aging process have been reported to date, partly owing to the hair-trigger rapid degeneration of hiPSC-Heps (Nie et al., 2018). In this paper, we established a simple and convenient method for generation of hiPSC-Heps that can maintain hepatic function for a long time with gradual accumulation of aging markers and characteristics. We noticed that the plasticity of hiPSC-Heps significantly decreased with aging-associated histone hypoacetylation. Moreover, treatment with histone deacetylase inhibitors (HDACi) obviously increased the plasticity of old hiPSC-Heps and PHHs, and this effect markedly improved the regenerative capacity of old PHHs in a mouse model of liver injury.

### Results

Convenient Generation of hiPSC-Heps with Improved Function

To prepare a hepatic aging model using hiPSC-Heps, we developed a two-step method for generation of hiPSC-Heps by drawing on our experience in the formation of hiPSC-derived liver organoids (Nie et al., 2018) (FIGS. 1-1A and 1-2A). At the first step, hiPSCs were efficiently differentiated into an endodermal-cell population of high purity (FIGS. 1-1B and 1-2B). At the second step, we induced the endodermal cells to directly differentiate into hepatocytes using a hepatic differentiation medium (HDM) containing screened hepatic growth and differentiation factors and found that these lineages matured into hepatocytes with ALB expression (FIGS. 1-2C and 1-2D).

During the progression from endodermal cells to hepatocytes, these lineages routinely showed trait changes characteristic of hepatocytes and maturation with increasing albumin (ALB) secretion (Si-Tayeb et al., 2010) (FIGS. 1-1A-C and 1-2E). After 5 days of differentiation of endodermal cells, these lineages manifested rapidly increasing expression of early hepatic-fate genes and sharply decreasing expression of endoderm-related genes (FIG. 1-2B). Immunostaining revealed that these cells were at the hepatoblast stage with positive expression of AFP, CK19, HNF4A, and KI67 and modest ALB secretion (FIGS. 1-1C and 1-2E). After another 10 days of incubation, these lineages matured into ALB-expressing cells (FIGS. 1-1B and 1-1C), showing expression of tight junction proteins E-cadherin (E-CAD) and zona occludens 1 (ZO-1; FIG. 1-2F) as well as hepatic functions, such as ICG uptake and release, glycogen storage, and low-density lipoprotein uptake (FIG. 1-2G). In comparison with hiPSC-Heps obtained via existing protocols (K-Heps and ST-Heps) (Kajiwara et al., 2012; Si-Tayeb et al., 2010), these newly generated hiPSC-Heps (N-Heps) are characterized by enhanced expression of liver-specific genes as well as ALB secretion (FIGS. 1-2H and 1-21). Thus, this study offers a simple and convenient protocol for generation of hiPSC-Heps with improved hepatic functions.

### Spontaneous Recapitulation of the Hepatic Aging Process

In contrast to rapid degeneration of previously reported hiPSC-Heps (Nie et al., 2018), the N-Heps retained cellular morphology and hepatic functions for a long time (FIGS. 1-1D and 1-2J). Namely, up to 72 days of cell culture, we observed formation of multinucleated cells with a decline of ALB secretion (FIGS. 1-1D and 1-2J). On day 82 (D82), N-Heps lost their hepatocyte morphology and showed a high proportion of apoptotic cells (FIG. 1-2K). Meanwhile, we noticed that the cell volume of N-Heps was enlarging during culture, a phenomenon frequently observed during cellular aging (Neurohr et al., 2019) (FIG. 1-2L). In order to test whether the N-Heps went through a process of aging, we performed transcriptome analysis to compare D22-Hep (hepatocyte that developed from hiPSCs after 22 days), D52-Hep, and D72-Hep and found that aging-related genes were abundantly expressed in D72-Heps (FIG. 1-1E). Gene ontology analysis indicated that aging-related hepatic changes (Kim et al., 2015) (including an increased inflammatory response and DNA damage) were also enhanced in D72-Hep (FIG. 1-2M). Moreover, the levels of intracellular reactive oxygen species (ROS) and senescence-associated β-galactosidase (SA-β-gal) (which were two cellular hallmarks of aging) gradually increased from D22 to D72 (FIGS. 1-1F-H). These results indicated spontaneous aging of N-Heps during prolonged cultivation.

Next, we wondered whether this aging process of N-Hep could recapitulate the in vivo hepatocyte aging process. By comparing the transcriptional differences among D22-Hep, D72-Hep, young PHH (2 months, 2M-PHH), and elderly PHH (78 years, 78Y-PHH), principal component analysis showed that the gene signatures of D22-Hep are similar to those of 2M-PHH, and the gene signatures of D72-Hep are very close to those of 78Y-PHH (FIG. 1-3 A). Moreover, KEGG pathway analysis showed that 35 signal pathways experiencing increased expression during in vitro aging process are also enhanced in expression during in vivo aging process, and 35 signaling pathways experiencing decreased expression during in vitro aging process are also decreased in expression during in vivo aging process (FIGS. 1-3B and C). These results suggest that the aging process of N-Hep could represent (replace) the aging process of hepatocyte in vivo.

### The Essential Function of FGF2 in the Plasticity Induction of Human Hepatocytes

Next, we tested whether the N-Heps have the same plasticity as do hepatocytes in vivo. N-Heps can be induced to proliferate with a bipotential differentiation ability (FIG. 2-1A). To induce hepatocyte proliferation, we cultured young N-Hep (D22-Hep) using a basic medium containing a previously described small-molecule cocktail (BM+SMs) (which can induce proliferation of rodent hepatocytes) (Katsuda et al., 2017). Six days later, the reprogrammed cells did not acquire a continuous proliferative ability that is seen in reprogrammed rodent hepatocytes (Katsuda et al., 2017) (FIG. 2-2A) but we observed cellular proliferation (FIGS. 2-1B and C). These data indicated that inherent differences between rodent and human hepatocytes might have a significant influence on plasticity induction outcomes. We previously confirmed that FGF2 is a key factor promoting the differentiation and maturation of hepatocytes (FIGS. 2-2C and D) and is also known as an important cytokine in liver homeostasis and regeneration (Steiling et al., 2003). We found that through the addition of FGF2 to the culture medium, the induced proliferative ability greatly improved (FIGS. 2-1B, 2C, and 2-2B). Of note, cells reprogrammed via FGF2 acquired an ability to proliferate continuously in the above-mentioned medium called BM+SMs when it was supplemented with FGF2. We termed this FGF2-supplemented medium a reprogramming medium (RM; FIG. 2-2A). Quantitative PCR (Q-PCR) analyses revealed that these reprogrammed cells featured enhanced transcription of cell cycle genes (FIG. 2-1D), and immunofluorescence analyses indicated that the proliferative cells still belonged to the hepatocyte lineage because they were double positive for KI67 and HNF4A (FIG. 2-1E). Therefore, we named these reprogrammed cells (hepatic progenitor cells) as proliferative hepatic cells (pHCs). Time-lapse imaging suggested that pHCs could be obtained from a single hepatocyte, as was the case for other hiPSC-Heps (FIGS. 2-2C and D). Compared with D22-Hep, D22-pHC showed higher transcription of hepatic-stem/progenitor cell—related genes (FIG. 2-1F) and downregulation of hepatic-function-related genes (FIG. 2-1G). Moreover, D22-pHC manifested positive staining for HNF4A, SOX9, and CK19 but were not stained for ALB and AFP (FIGS. 2-1H, 2-2E, and 2-2F).

In order to determine how FGF2 caused the induction of hepatic-cell proliferation, we incubated the cells with PD0325901 and LY294002 to respectively block MAPK and PI3K signaling pathways, which are the signaling cascades downstream of FGF2 and are involved in cell proliferation (Goetz and Mohammadi, 2013). Of note, PD0325901 rather than LY294002 inhibited the FGF2-mediated cellular proliferation in a dose-dependent manner (FIG. 2-11). Combining transcriptome and protein-protein interaction network analyses (Jensen et al., 2009), we found that EZH2 was at the central node of all the upregulated genes and linked the gene cluster of the cell cycle and the gene cluster of epithelial-mesenchymal transition (FIG. 2-2G). Moreover, RM treated cells showed obvious upregulation of EZH2 transcription that was suppressed by PD0325901 (FIGS. 2-1J and 2-2H). By disabling EZH2 function with its specific inhibitor, 3-deazaneplanocin A hydrochloride (DZNep), we detected significant attenuation of the induced cellular proliferation with only slight changes in EZH2 transcription magnitude (FIGS. 2-21 and J). This indicates that the FGF2-MAPK axis-activated EZH2 is strongly involved in the induced proliferation of human hepatocytes. Additionally, loss- and gain-of-function experiments suggested that the FGF2-EZH2 axis was indispensable for the maintenance of the proliferative ability of pHC (with a population doubling time of 19.86±0.70 hr) (FIGS. 2-2K and L), Prolonged proliferation revealed that these D22-pHC could be passaged at least 20 times without showing any obvious changes in their morphology, proliferation rate, and karyotype (FIGS. 2-2M and N). Thus, we successfully induced cellular proliferation of N-Heps that is controlled by the FGF2-MAPK-EZH2 axis (FIG. 2-1K).

### Bipotential Differentiation of D22-pHC

Next, we evaluated bipotential differentiation of pHC. To induce hepatocyte differentiation, we cultured D22-pHC in the newly developed HDM. After 15 days, the differentiated cells showed limited ALB production and atypical morphology (FIGS. 3-2A and B). To enhance the differentiation process, we modified HDM by adding retinoic acid (RA; FIG. 3-1A), a metabolite of vitamin A mediating liver development and regeneration (Negishi et al., 2010), and finally obtained a significant improvement in ALB production and typical hepatocyte morphology (FIGS. 3-2A-C). D22-pHC derived hepatocytes (D22-pHC-Hep) showed an obvious enhancement of the expression of functional genes as well as ALB secretion, which were equivalent to those of original D22-Hep (FIGS. 3-1B, C, and 3-2D). Moreover, D22-pHC-Hep formed tight cellular junctions between themselves and acquired the capacity for ammonia clearance and hepatic functions (ICG uptake and release, glycogen storage, and low-density lipoprotein uptake) (FIGS. 3-1D, E and 3-2E).

To induce differentiation into cholangiocytes, we employed the process of bile duct development and differentiation (Francis et al., 2004; Sampaziotis et al., 2015; Sampaziotis et al., 2017) and established a method of differentiation into cholangiocytes as presented in FIG. 3-1F. After 2 days of incubation, a single D22-pHC grew into a ring-like structure and then gradually developed into a cyst structure within 10 days (FIGS. 3-1F, 3-2F, and G). According to Q-PCR analysis, the cyst structure featured upregulated transcription of cholangiocyte signature genes (FIGS. 3-1G and 3-2H). The cholangiocyte fate of these cysts was then confirmed by positive results of CK19, F-ACTIN, and SOX9 staining and negative results of AFP, ALB, and HNF4A staining (FIG. 3-1H). Moreover, transmission electron microscopy indicated that these cysts possessed mature bile duct features (including a lumen with an apical and basolateral plasma membrane, microvilli, primary cilia on the apical membrane, tight junctions, and multivesicular bodies) (FIGS. 3-11 and 3-21). Meanwhile, these cholangiocytes had bile duct functions: 1) transport of Rhodamine 123 into the lumen; this function was blocked by verapamil, an inhibitor of multi-drug resistance protein 1 transporter (FIGS. 3-1J and K), and 2) specifically exporting a bile acid from the lumen (FIG. 3-2J). Therefore, these data revealed that D22-Hep (young N-Hep) had sufficient plasticity to enable induction of a proliferative status with bipotential differentiation.

### Impairment of Hepatocyte Plasticity in the Aging Process

In order to determine the intrinsic connections between aging and hepatocyte plasticity, we used the newly developed RM to induce plasticity of N-Heps in the aging process (FIG. 4-1A). Six days later, we observed a dramatic decline of induced pHC's cellular proliferation during the progression of aging from D22-Hep to D82-Hep (FIGS. 4-1B and C). Compared with the massively induced pHC's proliferation in D22-Hep, despite the use of the same procedure, only a few proliferative hepatocyte (D52-pHC) colonies were detected among reprogrammed D52-Hep (FIGS. 4-1B and 4-2A), and almost no proliferative cells/colonies were induced in D62-, D72-, and D82-Hep (FIGS. 4-1B and 4-2B). We then performed transcriptomic and Kyoto Encyclopedia of Genes and Genomes (KEGG) pathway analyses to figure out the alterations in N-Heps during aging. Remarkably, the aging N-Heps were characterized by a decline of metabolic pathways of fatty acids, glucose, and amino acids (FIGS. 4-2C and D) and manifested significant downregulation of acetyl-coenzyme A (CoA) which is the central intermediate of these metabolic pathways (FIGS. 4-1D and 4E). Acetyl-CoA is also a key node linking metabolomics to epigenetics as the acetyl source for histone acetylation (Sebastian and Mostoslaysky, 2017). Hence, we quantitatively analyzed the alterations in histone acetylation with aging. Of note, the aging N-Heps showed a downward trend in acetylation of H3 histone at Lys residue of each of H3K9, H3K18 and H3K27 (FIGS. 4-1F and 4-2E-G). On the other hand, these three types of histone modification by acetylation clearly correlated with their induced pHC's proliferative ability (FIG. 4-1G). These findings suggest that aging-associated metabolic defects are connected with histone hypoacetylation, which might impair hepatocyte plasticity (FIG. 4-2H).

### Upregulation of the Plasticity of Old N-Heps by HDACi

Next, to investigate whether histone hypoacetylation would cause the impairment of hepatocyte plasticity with aging, we stimulated D52-Heps with sodium butyrate (NaB) or valproic acid (VPA) (both being a histone deacetylase inhibitor (HDACi)) in the induction process and found that each HDACi markedly promoted the proliferation of pHCs induced from D52-Heps (FIGS. 4-1H, I, and 4-3A). Immunostaining indicated that the stimulation by a single HDACi significantly increased the percentage of HNF4⁺KI67⁺ cells (FIG. 4-1J). Meanwhile, such promotion was not detectable during the stimulation with other types of epigenetic regulators or inhibitors such as tranylcypromine (Trany, a histone demethylase inhibitor), RG108 (a DNA methyltransferase inhibitor), and BIX01294 (BIX, a histone methyltransferase inhibitor (FIGS. 4-1H and I). Moreover, it was clear that D52-pHC (D52-Hep-derived pHC) acquired the abilities to proliferate continuously and differentiate into hepatocytes and cholangiocytes whereas stimulation with HDACi improved the plasticity of D52-Hep (FIGS. 4-3B-F).

In one of the above experiments, we found that FGF2-activated EZH2 transcription plays a decisive role in the proliferation of proliferative hepatic cells (FIGS. 2-lI, J, and 2-2G-J), whereas the activated transcription of EZH2 was much lower in reprogrammed D52-Hep (D52-pHC) than in reprogrammed D22-Hep (D22-pHC) (FIG. 4-3G). Meanwhile, the stimulation by HDACi enhanced EZH2 transcription to promote the proliferation of D52-pHC induced from D52-Hep (FIGS. 4-1K and 4-3H), and D52-pHC showed greater histone acetylation as compared with D52-Hep (FIG. 4-31). These data suggested that histone hypoacetylation might be involved in the regulation of EZH2 transcription. In order to study the EZH2 promoter, we performed a comparative analysis using a comprehensive web tool (https://www.genecards.org) and ENCODE data sets (Consortium, 2012), whereupon we found that the two EZH2 enhancers with the highest score (GH07J148882 and GH07J148940) were the binding sites of the acetylated histone (FIG. 4-3J). Furthermore, according to chromatin immunoprecipitation with PCR analysis (ChIP-PCR) based on anti-H3K9ac, anti-H3K18ac and anti-H3K27ac antibodies, D52-Hep showed a significant decrease of acetylated-histone binding to GH07J148882 (which is close to the transcription start site) (FIGS. 4-1L and 4-3J). These data implied that histone hypoacetylation resulted in insufficient activation of EZH2 transcription and that this problem would impede the induction of plasticity in the old hepatocytes. Moreover, stimulation with an HDACi enhanced the proliferation of the respective pHC induced from D62-Hep, D72-Hep, and D82-Hep (FIG. 4-1M). These results illustrated that histone hypoacetylation-mediated inactivation of EZH2 transcription impairs the plasticity of old hepatocytes and that this plasticity can be effectively recovered with HDACi (FIG. 4-3K).

### Induction of Plasticity of PHHs

We next tested whether the mechanism of action uncovered in N-Heps is consistent with that in PHHs. By analyzing histone acetylation in PHHs derived from donors at ages of 2 months (2M-PHH), 39 years (39Y-PHH) and 78 years (78Y-PHH), we noticed that the amounts of H3K9ac, H3K18ac, and H3K27ac also decreased with aging (FIG. 5-1A). In comparison with N-Heps, the acetylation levels of 2M-PHH were close to those of D52-Hep, and the acetylation levels of 39Y-PHH and 78Y-PHH were lower than those of D62-Hep (FIG. 5-1B). Indeed, the aging-associated hypoacetylation in PHHs was also accompanied by a decline of plasticity induction, whereas stimulation with NaB (HDACi) significantly increased the plasticity induction efficiency (FIGS. 5-1C, 5-1D, and 5-2A). According to a transcriptomic comparison with original PHHs, PHH-derived pHC (PHH-pHC) showed an increase in the expression levels of cell cycle-related and stem/progenitor cell-related signature genes and a decrease in the expression levels of hepatic functional signature genes (FIGS. 5-1E, F, and 5-2B). Immunofluorescence analyses indicated that 49.8% (2M-pHC), 32.9% (39Y-pHC), and 12.2% (78Y-pHC) of PHH-pHCs were double-positive for the expression of HNF4A and KI67, and most of the PHH-pHCs expressed HNF4A, SOX9, and CK19 (FIG. 5-1G). Moreover, PHH-pHC manifested a proliferative ability on a laminin-511-coated culture dish (FIG. 5-2C) and showed bipotential differentiation to be capable of differentiating into hepatocytes and cholangiocytes (FIGS. 5-2D-J). These results indicated that the aging process of PHHs, similar to that of N-Heps, was also associated with histone hypoacetylation that impaired hepatocyte plasticity. These findings confirmed the usefulness of N-Heps as a cell source for studies on human hepatic aging.

In Vivo Repopulation of pHCs after Plasticity Induction of Old PHHs

Next, we evaluated the repopulation of old PHH-pHC in the liver using TK-NOG mice after ganciclovir-induced liver failure (Hasegawa et al., 2011). At 4 weeks after transplantation, the mice transplanted with 78Y-PHH or 78Y-pHC tested positive in the assays of human ALB (hALB), human A1AT (hA1AT), and human ferritin (hFerritin), but few tested positive for human AFP (hAFP; FIGS. 6-1 A-C and 6-2A).

In contrast to the significant gaps of hALB, hA1AT and hFerritin between transplants 2M-PHH and 78Y-PHH, little gap was seen between the 78Y-pHC transplant and the 2M-pHC transplant, with levels being much higher than the corresponding levels in 78Y-PHH to show production which was rather comparable to young primary hepatocytes (FIGS. 6-1A-C). As for hALB, detailed confirmation at the gene expression level was performed by Q-PCR (FIG. 6-2B). Moreover, immunofluorescence analyses suggested that the 78Y-pHC transplant yielded larger numbers of hALB+grafts and clusters (cell number per cluster >5) than did the 78Y-PHH transplant (FIGS. 6-1D, E, and 6-2C). Of note, over 50% of hALB+grafts in the 78Y-pHC transplant group expressed CK19, and this percentage was much higher than that in the 78Y-PHH transplant group (FIGS. 6-1D and F). Moreover, we detected some ALB+KI67+clusters in the 78Y-pHC transplant but none in the 78Y-PHH transplant (FIG. 6-2D).

Furthermore, hALB production in the 78Y-pHC transplant was much higher than the hALB production of the 78Y-PHH transplant (FIG. 6-1G). Because a significant decline in the expression of hepatic functional genes was seen during plasticity induction (FIGS. 5-2B and 6-2E), we investigated whether the transplanted PHH-pHC matured in vivo. At 12 weeks after transplantation, according to Q-PCR using human-gene-specific primers, the transplanted PHH-pHC showed a significant recovery in the expression of hepatic functional genes (especially CYP450 enzymes) and downregulation of CK19 (FIG. 6-2E). In immunofluorescence analyses, the grafts of PHH-pHCs showed positive results for the expression of hALB, CYP3A4, hNuMA, hA1AT, and hCK8/18 whereas hCK19 and hAFP were hardly expressed (FIGS. 6-1H and 6-2F). The positive result of ZO-1 staining indicated that PHH-pHC matured into a liver tissue featuring bile canaliculi formation and bipolar hepatocyte configuration (FIG. 6-1H). These results meant that plasticity induction performed using old PHHs markedly enhanced their repopulation ability and that they matured after transplantation to be capable of displaying adequate liver function.

HDAC Inhibitor Improves the Induction of pHCs in the Elderly Model.

Finally, we investigated whether HDACi could improve the induction of hepatic progenitor cells in elderly models. To induce hepatic progenitor cells in mice, we fed young and elderly mice with a choline-deficient, ethionine-supplemented (CDE) diet for 21 days. The detection of GOT and GPT showed that chronic liver injury occurred in the young and elderly mice, and the liver of mice models presented the characteristics of non-alcoholic fatty liver disease (FIGS. 6-3A-C). Moreover, the survival rate of the elderly model was only 60%, revealing that the induction of Ki67 positive cells and Epcam positive cells in the elderly model was significantly lower than that in the young model and these suggest that induction of hepatic progenitors in mice liver was also impaired with aging (FIGS. 6-3D-H). Upon NaB treatment, the induced Ki67 positive and Epcam positive population (cell population) significantly increased in the elderly model and the survival rate of the elderly model was also improved (FIGS. 6-3D-H).

### Discussion

Liver aging is a normal physiological process in which hepatocytes gradually lose the functions necessary for homeostasis as well as plasticity (Timchenko, 2009). The development of strategies against aging-associated liver diseases and for improvement of the hepatic regenerative capacity in the elderly has been hampered by the lack of understanding of the mechanisms of action by which aging regulates hepatocyte plasticity. By applying the hiPSC directional differentiation technology, we created young and old hepatocytes having the same genetic background. This arrangement helped us to reveal that aging-associated histone hypoacetylation impairs hepatocyte plasticity, On the other hand, upregulation of histone acetylation may significantly improve the plasticity of old hepatocytes. On the basis of this hypothesis, we successfully obtained pHC from old PHH (derived from a 78-year-old donor), and in a liver injury model, the pHC showed a greater repopulation ability than did the original old PHH.

Due to the shortage of donors for liver transplantation, there is a growing need for the use of grafts derived from elderly donors, which helps reduce the mortality linked to the waiting list, but the initial poor regenerative capacity of old hepatocytes may lead to graft failure and bad outcomes (Bernal and Wendon, 2013; Durand et al., 2019; Uemura et al., 2007). Accumulating evidence indicates that hepatocytes in grafts from the elderly yield a low proliferative response, and this disadvantage impairs liver regeneration (Ono et al., 2011; Schmucker and Sanchez, 2011). Compared with transplantation of young PHHs in our liver injury model, old-PHH transplants produced lower amounts of human hepatic proteins, thus reproducing the difference in clinical outcomes between young and old grafts. Notably, upregulation of histone acetylation markedly promoted proliferation of old PHHs, which manifested a significant improvement of regenerative capacity. This suggests that transplantation of grafts derived from the elderly (which had the proliferation capacity inherent in hepatocytes effectively activated or promoted) should improve graft survival and clinical outcomes.

The aging process is always characterized by dynamic alterations in metabolic processes and by epigenetic modifications (Peleg et al., 2016; Ren et al., 2017), whereas how these alterations regulate cellular functions remains a mystery in the field of liver research (Horvath et al., 2014; Sato et al., 2017). In this paper, we revealed close relations among aging, hepatocyte plasticity, metabolism, and epigenetic modifications. In particular, hepatocyte aging turned out to be accompanied by a decline of metabolic functions, which will affect the regulation of histone acetylation through acetyl-CoA, thereby causing an impairment of hepatocyte plasticity. In contrast to rodent hepatocytes (Katsuda et al., 2017), FGF2-activated EZH2 transcription was necessary to induce the plasticity of human hepatocytes, and hypoacetylation of an EZH2 enhancer in old hepatocytes was found to impair the plasticity of those cells. Some researchers noticed that calorie restriction in an old mouse liver could reverse the aging-dependent decline in histone acetylation and activate the transcription of cell cycle genes (Sato et al., 2017) but until this study was accomplished, there has been no suitable method for enhancing the acetylation of histone by improving cellular metabolism in a culture dish. Considering the reversibility of histone acetylation and deacetylation, we found that suppression of HDACs can greatly improve the plasticity of old hepatocytes and further confirmed the regulation of the plasticity of old hepatocytes by histone hypoacetylation to offer a feasible strategy for improving the plasticity of old hepatocytes.

In addition to compromising homeostasis and regulating the liver regeneration, aging also plays an important role in the development of liver diseases (Kim et al., 2015). The decline of mitochondrial function during aging has been reported to enhance vulnerability to injury (Kim et al., 2015), and aging is considered a predictor of undesirable outcomes of alcoholic hepatitis and of fibrosis progression in hepatitis C (Forrest et al., 2005; Poynard et al., 2001). In the present study, we demonstrated that hiPSC-Heps can reproduce the hepatic aging process. Meanwhile, our bioinformatics analyses indicate that old hiPSC-Heps tend to express genes that promote angiogenesis and fibrogenesis (FIG. 1-2M), suggesting that hiPSC-Heps would be useful for revealing the close links between aging and liver fibrosis and for developing effective therapeutic strategies against aging-related liver diseases.

In conclusion, we recapitulated the hepatocyte aging process in a culture dish and found that hepatocyte plasticity is impaired by aging-linked histone hypoacetylation. Additionally, promotion of histone acetylation improved the plasticity of old hepatocytes and enhanced the repopulation ability of those cells in the liver injury model, thereby pointing to a promising therapeutic strategy for promoting liver regeneration in the elderly.

### REFERENCES

Aikata, H., Takaishi, H., Kawakami, Y., Takahashi, S., Kitamoto, M., Nakanishi, T., Nakamura, Y., Shimamoto, F., Kajiyama, G., and Ide, T. (2000). Telomere reduction in human liver tissues with age and chronic inflammation. Experimental cell research 256, 578-582.
Bernal, W., and Wendon, J. (2013). Acute liver failure. The New England journal of medicine 369, 2525-2534.
Bucher, N. L., Swaffield, M. N., and Ditroia, J. F. (1964). The Influence of Age Upon the Incorporation of Thymidine-2-C14 into the DNA of Regenerating Rat Liver. Cancer research 24, 509-512.
Consortium, E. P. (2012). An integrated encyclopedia of DNA elements in the human genome. Nature 489, 57-74.
Deng, X., Zhang, X., Li, W., Feng, R. X., Li, L., Yi, G. R., Zhang, X. N., Yin, C., Yu, H. Y., Zhang, J. P., et al. (2018). Chronic Liver Injury Induces Conversion of Biliary Epithelial Cells into Hepatocytes. Cell stem cell 23, 114-122 e113.
Durand, F., Levitsky, J., Cauchy, F., Gilgenkrantz, H., Soubrane, O., and Francoz, C. (2019). Age and liver transplantation. Journal of hepatology 70, 745-758.
Forrest, E. H., Evans, C. D., Stewart, S., Phillips, M., Oo, Y. H., McAvoy, N. C., Fisher, N. C., Singhal, S., Brind, A., Haydon, G., et al. (2005). Analysis of factors predictive of mortality in alcoholic hepatitis and derivation and validation of the Glasgow alcoholic hepatitis score. Gut 54, 1174-1179.
Francis, H., Glaser, S., Ueno, Y., Lesage, G., Marucci, L., Benedetti, A., Taffetani, S., Marzioni, M., Alvaro, D., Venter, J., et al. (2004). cAMP stimulates the secretory and proliferative capacity of the rat intrahepatic biliary epithelium through changes in the PKA/Src/MEK/ERK1/2 pathway. Journal of hepatology 41, 528-537.
Fry, M., Silber, J., Loeb, L. A., and Martin, G. M. (1984). Delayed and reduced cell replication and diminishing levels of DNA polymerase-alpha in regenerating liver of aging mice. Journal of cellular physiology 118, 225-232.
Fu, G. B., Huang, W. J., Zeng, M., Zhou, X., Wu, H. P., Liu, C. C., Wu, H., Weng, J., Zhang, H. D., Cai, Y. C., et al. (2019). Expansion and differentiation of human hepatocyte-derived liver progenitorlike cells and their use for the study of hepatotropic pathogens. Cell research 29, 8-22.
Germani, G., Theocharidou, E., Adam, R., Karam, V., Wendon, J., O'Grady, J., Burra, P., Senzolo, M., Mirza, D., Castaing, D., et al. (2012). Liver transplantation for acute liver failure in Europe: outcomes over 20 years from the ELTR database. Journal of hepatology 57, 288-296.
Goetz, R., and Mohammadi, M. (2013). Exploring mechanisms of FGF signalling through the lens of structural biology. Nature reviews Molecular cell biology 14, 166-180.
Hasegawa, M., Kawai, K., Mitsui, T., Taniguchi, K., Monnai, M., Wakui, M., Ito, M., Suematsu, M., Peltz, G., Nakamura, M., et al. (2011). The reconstituted ‘humanized liver’ in TK-NOG mice is mature and functional. Biochemical and biophysical research communications 405, 405-410.
Horvath, S., Erhart, W., Brosch, M., Ammerpohl, O., von Schonfels, W., Ahrens, M., Heits, N., Bell, J. T., Tsai, P. C., Spector, T. D., et al. (2014). Obesity accelerates epigenetic aging of human liver. Proceedings of the National Academy of Sciences of the United States of America 111, 15538-15543.
Iakova, P., Awad, S. S., and Timchenko, N. A. (2003). Aging reduces proliferative capacities of liver by switching pathways of C/EBPalpha growth arrest. Cell 113, 495-506.
Jensen, L. J., Kuhn, M., Stark, M., Chaffron, S., Creevey, C., Muller, J., Doerks, T., Julien, P., Roth, A., Simonovic, M., et al. (2009). STRING 8—a global view on proteins and their functional interactions in 630 organisms. Nucleic acids research 37, D412-416.
Kajiwara, M., Aoi, T., Okita, K., Takahashi, R., Inoue, H., Takayama, N., Endo, H., Eto, K., Toguchida, J., Uemoto, S., et al. (2012). Donor-dependent variations in hepatic differentiation from humaninduced pluripotent stem cells. Proceedings of the National Academy of Sciences of the United States of America 109, 12538-12543.
Katsuda, T., Kawamata, M., Hagiwara, K., Takahashi, R. U., Yamamoto, Y., Camargo, F. D., and Ochiya, T. (2017). Conversion of Terminally Committed Hepatocytes to Culturable Bipotent Progenitor Cells with Regenerative Capacity. Cell stem cell 20, 41-55.
Kim, I. H., Kisseleva, T., and Brenner, D. A. (2015). Aging and liver disease. Current opinion in gastroenterology 31, 184-191.
Kim, Y., Kang, K., Lee, S. B., Seo, D., Yoon, S., Kim, S. J., Jang, K., Jung, Y. K., Lee, K. G., Factor, V. M., et al. (2019). Small molecule-mediated reprogramming of human hepatocytes into bipotent progenitor cells. Journal of hepatology 70, 97-107.
Kopp, J. L., Grompe, M., and Sander, M. (2016). Stem cells versus plasticity in liver and pancreas regeneration. Nature cell biology 18, 238-245.
Li, D., Li, W., and Hui, L. (2016). Hybrid hepatocyte: A newly identified player for regeneration in hepatic injuries. Hepatology 64, 2244-2246.
Miller, J. D., Ganat, Y. M., Kishinevsky, S., Bowman, R. L., Liu, B., Tu, E. Y., Mandal, P. K., Vera, E., Shim, J. W., Kriks, S., et al. (2013). Human iPSC-based modeling of late-onset disease via progerin-induced aging. Cell stem cell 13, 691-705.
Negishi, T., Nagai, Y., Asaoka, Y., Ohno, M., Namae, M., Mitani, H., Sasaki, T., Shimizu, N., Terai, S., Sakaida, I., et al. (2010). Retinoic acid signaling positively regulates liver specification by inducing wnt2bb gene expression in medaka. Hepatology 51, 1037-1045.
Neurohr, G. E., Terry, R. L., Lengefeld, J., Bonney, M., Brittingham, G. P., Moretto, F., Miettinen, T. P., Vaites, L. P., Soares, L. M., Paulo, J. A., et al. (2019). Excessive Cell Growth Causes Cytoplasm Dilution And Contributes to Senescence. Cell 176, 1083-1097 e1018.
Nie, Y. Z., Zheng, Y. W., Miyakawa, K., Murata, S., Zhang, R. R., Sekine, K., Ueno, Y., Takebe, T., Wakita, T., Ryo, A., et al. (2018). Recapitulation of hepatitis B virus-host interactions in liver organoids from human induced pluripotent stem cells. EBioMedicine 35, 114-123.
Ono, Y., Kawachi, S., Hayashida, T., Wakui, M., Tanabe, M., Itano, O., Obara, H., Shinoda, M., Hibi, T., Oshima, G., et al. (2011). The influence of donor age on liver regeneration and hepatic progenitor cell populations. Surgery 150, 154-161.
Peleg, S., Feller, C., Ladurner, A. G., and Imhof, A. (2016). The Metabolic Impact on Histone Acetylation and Transcription in Ageing. Trends in biochemical sciences 41, 700-711.
Poynard, T., Ratziu, V., Charlotte, F., Goodman, Z., McHutchison, J., and Albrecht, J. (2001). Rates and risk factors of liver fibrosis progression in patients with chronic hepatitis c. Journal of hepatology 34, 730-739.
Ren, R., Ocampo, A., Liu, G. H., and Izpisua Belmonte, J. C. (2017). Regulation of Stem Cell Aging by Metabolism and Epigenetics. Cell metabolism 26, 460-474.
Sampaziotis, F., de Brito, M. C., Madrigal, P., Bertero, A., Saeb-Parsy, K., Soares, F. A. C., Schrumpf, E., Melum, E., Karlsen, T. H., Bradley, J. A., et al. (2015). Cholangiocytes derived from human induced pluripotent stem cells for disease modeling and drug validation. Nature biotechnology 33, 845-852.
Sampaziotis, F., Justin, A. W., Tysoe, O.C., Sawiak, S., Godfrey, E. M., Upponi, S. S., Gieseck, R. L., 3rd, de Brito, M. C., Berntsen, N. L., Gomez-Vazquez, M. J., et al. (2017). Reconstruction of the mouse extrahepatic biliary tree using primary human extrahepatic cholangiocyte organoids. Nature medicine 23, 954-963.
Sato, S., Solanas, G., Peixoto, F. O., Bee, L., Symeonidi, A., Schmidt, M. S., Brenner, C., Masri, S., Benitah, S. A., and Sassone-Corsi, P. (2017). Circadian Reprogramming in the Liver Identifies Metabolic Pathways of Aging. Cell 170, 664-677 e611.
Sawada, N. (1989). Hepatocytes from old rats retain responsiveness of c-myc expression to EGF in primary culture but do not enter S phase. Experimental cell research 181, 584-588.
Schmucker, D. L., and Sanchez, H. (2011). Liver regeneration and aging: a current perspective. Current gerontology and geriatrics research 2011, 526379.
Sebastian, C., and Mostoslaysky, R. (2017). The Various Metabolic Sources of Histone Acetylation. Trends in endocrinology and metabolism: TEM 28, 85-87.
Si-Tayeb, K., Noto, F. K., Nagaoka, M., Li, J., Battle, M. A., Duris, C., North, P. E., Dalton, S., and Duncan, S. A. (2010). Highly efficient generation of human hepatocyte-like cells from induced pluripotent stem cells. Hepatology 51, 297-305.
Stadtfeld, M., and Hochedlinger, K. (2010). Induced pluripotency: history, mechanisms, and applications. Genes & development 24, 2239-2263.
Steiling, H., Wustefeld, T., Bugnon, P., Brauchle, M., Fassler, R., Teupser, D., Thiery, J., Gordon, J. I., Trautwein, C., and Werner, S. (2003). Fibroblast growth factor receptor signalling is crucial for liver homeostasis and regeneration. Oncogene 22, 4380-4388.
Studer, L., Vera, E., and Cornacchia, D. (2015). Programming and Reprogramming Cellular Age in the Era of Induced Pluripotency. Cell stem cell 16, 591-600.
Tarlow, B. D., Pelz, C., Naugler, W. E., Wakefield, L., Wilson, E. M., Finegold, M. J., and Grompe, M. (2014). Bipotential adult liver progenitors are derived from chronically injured mature hepatocytes. Cell stem cell 15, 605-618.
Timchenko, N. A. (2009). Aging and liver regeneration. Trends in endocrinology and metabolism: TEM 20, 171-176.
Uemura, T., Randall, H. B., Sanchez, E. Q., Ikegami, T., Narasimhan, G., McKenna, G. J., Chinnakotla, S., Levy, M. F., Goldstein, R. M., and Klintmalm, G. B. (2007). Liver retransplantation for primary nonfunction: analysis of a 20-year single-center experience. Liver transplantation: official publication of the American Association for the Study of Liver Diseases and the International Liver Transplantation Society 13, 227-233.
Vera, E., Bosco, N., and Studer, L. (2016). Generating Late-Onset Human iPSC-Based Disease Models by Inducing Neuronal Age-Related Phenotypes through Telomerase Manipulation. Cell reports 17, 1184-1192.
Yanger, K., Knigin, D., Zong, Y., Maggs, L., Gu, G., Akiyama, H., Pikarsky, E., and Stanger, B. Z. (2014). Adult hepatocytes are generated by self-duplication rather than stem cell differentiation. Cell stem cell 15, 340-349.
Yanger, K., Zong, Y., Maggs, L. R., Shapira, S. N., Maddipati, R., Aiello, N. M., Thung, S. N., Wells, R. G., Greenbaum, L. E., and Stanger, B. Z. (2013). Robust cellular reprogramming occurs spontaneously during liver regeneration. Genes & development 27, 719-724.
Zhang, K., Zhang, L., Liu, W., Ma, X., Cen, J., Sun, Z., Wang, C., Feng, S., Zhang, Z., Yue, L., et al. (2018). In Vitro Expansion of Primary Human Hepatocytes with Efficient Liver Repopulation Capacity. Cell stem cell 23, 806-819 e804.

### Key Resources Table

TABLE 1

| | | |
|---|---|---|
| REAGENT or RESOURCE | SOURCE | IDENTIFIER |

Antibodies

| | | |
|---|---|---|
| Goat anti-human ALB | Bethyl | Cat# [symbol] , RRID [symbol] AB_ [symbol] |
| Rabbit anti-human AFP | Agilent | Cat# [symbol] , RRID [symbol] AB_ [symbol] |
| Mouse anti-human CK19 | Agilent | Cat# [symbol] , RRID [symbol] AB_ [symbol] |
| Rabbit anti-human A1AT | Agilent | Cat# [symbol] , RRID [symbol] AB_ [symbol] |
| Chicken anti-human A1AT | GenWay Biotech Inc | Cat# [symbol] , RRID [symbol] AB_ [symbol] |
| Mouse anti-human KI67 | Agilent | Cat# [symbol] , RRID [symbol] AB_ [symbol] |
| Goat anti-human HNF4A | Santa Cruz Biotechnology | Cat# [symbol] , RRID [symbol] AB_ [symbol] |
| Mouse anti-human E-cadherin | BD Biosciences | Cat# [symbol] , RRID [symbol] AB_ [symbol] |
| Mouse anti-Z [symbol] -1 | Thermo Fisher Scientific | Cat# [symbol] , RRID [symbol] AB_ [symbol] |
| Mouse anti-human SOX9 | Abcam | Cat# [symbol] , RRID [symbol] AB_ [symbol] |
| Mouse anti-Human NuMA | Santa Cruz Biotechnology | Cat# [symbol] , RRID [symbol] AB_ [symbol] |
| Guinea Pig Ans-CK8/18 | ARP | Cat# [symbol] , RRID [symbol] AB_ [symbol] |
| Rabbit anti-H3K9ac | Cell Signaling Technology | Cat# [symbol] , RRID [symbol] AB_ [symbol] |
| Rabbit anti-H3K18ac | Cell Signaling Technology | Cat# [symbol] , RRID [symbol] AB_ [symbol] |
| Rabbit anti-H3K27ac | Cell Signaling Technology | Cat# [symbol] , RRID [symbol] AB_ [symbol] |
| Rabbit anti-H3K14ac | Cell Signaling Technology | Cat# [symbol] , RRID [symbol] AB_ [symbol] |
| Rabbit anti-H3K56ac | Cell Signaling Technology | Cat# [symbol] , RRID [symbol] AB_ [symbol] |
| PE Mouse Anti-Human CXCR4 | BD Biosciences | Cat# [symbol] , RRID [symbol] AB_ [symbol] |
| BV421 Mouse Anti-Human CD117 | BD Biosciences | Cat# [symbol] , RRID [symbol] AB_ [symbol] |
| APC Mouse Anti-Human EpCAM | Mr [symbol] Biotech | Cat# [symbol] , RRID [symbol] AB_ [symbol] |
| Donkey Anti-Mouse IgG (H + L) Alexa Fluor 488 | Thermo Fisher Scientific | Cat# A- [symbol] , RRID [symbol] AB_ [symbol] |
| Donkey Anti-Rabbit IgG (H + L) Alexa Fluor 488 | Thermo Fisher Scientific | Cat# A- [symbol] , RRID [symbol] AB_ [symbol] |
| Donkey Anti-Rabbit IgG (H + L) Alexa Fluor 555 | Thermo Fisher Scientific | Cat# A- [symbol] , RRID [symbol] AB_ [symbol] |
| Donkey Anti-Goat IgG (H + L) Alexa Fluor 555 | Thermo Fisher Scientific | Cat# A- [symbol] , RRID [symbol] AB_ [symbol] |
| Goat Anti-Guinea Pig IgG (H + L) Alexa Fluor 555 | Thermo Fisher Scientific | Cat# A- [symbol] , RRID [symbol] AB_ [symbol] |
| Goat Anti-Chicken IgY (H + L) Alexa Fluor 488 | Thermo Fisher Scientific | Cat# A- [symbol] , RRID [symbol] AB_ [symbol] |
| Goat Anti-Rabbit IgG (H + L) Alexa Fluor 647 | Thermo Fisher Scientific | Cat# A- [symbol] , RRID [symbol] AB_ [symbol] |

Chemicals, Peptides, and Recombinant Proteins

| | | |
|---|---|---|
| mTeSR1 | STEM CELL | Cat# [symbol] |
| StemF [symbol] | [symbol] | Cat# [symbol] |
| Iscove’a Modified Dulbecco's Medium ( [symbol] MDM) | Invitrogen | Cat# 12440- [symbol] |
| Ham's [symbol] -12K Medium (F [symbol] 2) | Invitrogen | Cat# [symbol] |

[symbol]

indicates data missing or illegible when filed

TABLE 2

| | | |
|---|---|---|
| RMP [symbol] 640 Medium | Wako | Cat# 189-02025 |
| [symbol] -27 ™ Supplement (50X) | Invitrogen | Cat# 17504-001 |
| N- [symbol] Supplement (100X) | Invitrogen | Cat# 175020-01 |
| Recombinant Human WNT3A | R&D Systems | Cat# [symbol] |
| Recombinant Human Activin A | [symbol] | Cat# P08476 |
| CultureSure ® Y-27632 | Wako | Cat# 034-24024 |
| Sodium butyrate (NaB) | Wako | Cat# [symbol] |
| Nicotinamide | Sigma | Cat# [symbol] |
| Recombinant Human FGF2 | Wako | Cat# [symbol] |
| Human recombinant HGF | Stemgent | Cat# 03-0019 |
| Recombinant Human Onc [symbol] M (OSM) | R&D Systems | Cat# [symbol] |
| Dexametha [symbol] one-Water Soluble | Sigma | Cat# [symbol] |
| AB [symbol] | R&D Systems | Cat# [symbol] |
| CH [symbol] | Cayman | Cat# 13122 |
| LY29002 | Sigma | Cat# L9908 |
| P [symbol] | Sigma | Cat# P20162 |
| 3-D [symbol] A (DZNep) | Sigma | Cat# [symbol] |
| Valproic acid (VPA) | Sigma | Cat# P4543 |
| Tran [symbol] (Trany) | ENZO | Cat# BML_EI217-0001 |
| RG10 [symbol] | Stemgent | Cat# 04-0001 |
| BIX012 [symbol] (BIX) | Cayman | Cat# [symbol] |
| Matrigel ® Growth Factor Reduced | CORNING | Cat# 154230 |
| Laminin-511 | Nippi | Cat# 381-67363 |
| ECL prime blocking agent | Roche | Cat# RPN416 |
| Trion X-100 | Sigma | Cat# [symbol] |
| Hepatocyte Culture Media [symbol] Kit ™ (HCM) | Lonza | Cat# CC-3198 |
| [symbol] LDL | Thermo Fisher Scientific | Cat# L3484 |
| [symbol] 33342 | Thermo Fisher Scientific | Cat# [symbol] |
| [symbol] trans-Retinic Acid (RA) | Wako | Cat# [symbol] |
| Recombinant Human EGF | Sigma | Cat# [symbol] |
| Recombinant Human R-Sp [symbol] | R&D Systems | Cat# [symbol] |
| Recombinant Human [symbol] | R&D Systems | Cat# [symbol] |
| [symbol] | Cayman | Cat# [symbol] |
| Ch [symbol] | CORNING | Cat# [symbol] |
| Rhodamine 123 | Sigma | Cat# 8 [symbol] |
| Fluorescein isothiocyanate (FITC) | Sigma | Cat# F [symbol] |

[symbol]

indicates data missing or illegible when filed

TABLE 3

| | | |
|---|---|---|
| [symbol] ,6-diamidino 2- [symbol] (DAPI) | D [symbol] | Cat# D [symbol] |
| CELLECT ™ Fetal Bovine Serum (FBS) | M [symbol] Biomedicals | Cat# [symbol] |

Critical Commercial Assays

| | | |
|---|---|---|
| Cellular [symbol] Detection Quanti [symbol] Kit | DOJINDO | Cat# [symbol] |
| PureLink ™ [symbol] NA [symbol] Kit | Thermo Fisher Scientific | Cat# [symbol] |
| Human Albumin ELISA Quant [symbol] Set | Bethyl Laboratories | Cat# [symbol] |
| CellROX ™ Deep Red Reagent | Thermo Fisher Scientific | Cat# [symbol] |
| [symbol] (PAS) staining kit | M [symbol] Pure Chemicals | Cat# [symbol] |
| SimpleCh [symbol] Enzymatic [symbol] IP Kit | Cell Signaling Technology | Cat# [symbol] |
| High-Capacity cDNA Reverse Transcription Kit | Thermo Fisher Scientic | Cat# [symbol] |
| Acetyl-Coanzyme-A Assay Kit | Sigma | Cat# [symbol] |
| Human Alpha [symbol] Amitrypsin ELISA Guanti [symbol] Kit | [symbol] | Cat# [symbol] |
| Human AFP ELISA Kit | [symbol] | Cat# [symbol] |
| Human Ferritin ELISA Kit | Abcam | Cat# [symbol] |

Experimental Models: Cell Lines

| | | |
|---|---|---|
| TKDA | Gift from Dr. Hiromitsu Nakauchi | |
| M [symbol] | Gift from Dr. Shinya Yamanaka | |
| 13 [symbol] | Gift from Dr. Shinya Yamanaka | |
| 13 [symbol] | Gift from Dr. Shinya Yamanaka | |
| YCU [symbol] | ( [symbol] et [symbol] ) | |
| Primary human hepatocyte (2 months) | BioreclamationIVT | Cat# 00995-P, TLY |
| Primary human hepatocyte (3 [symbol] years) | BioreclamationIVT | Cat# F [symbol] |
| Primary human hepatocyte (78 years) | BioreclamationIVT | Cat# F [symbol] |

Experimental Models: Organisms/Strains

| | |
|---|---|
| TK-NDG [symbol] | [symbol] |

Deposited Dat

[symbol]

| | |
|---|---|
| Microarray dat [symbol] | This paper |

Oligonucleotides

| | | |
|---|---|---|
| P [symbol] for [symbol] -PCR | This paper | N/A |

Software and Algorithms

| | |
|---|---|
| Graphpad Prism 5.0 | GrapAed software |
| [symbol] mage [symbol] | [symbol] |
| SigmaPlot [symbol] | [symbol] |
| GeneSpring | Agilent |

[symbol]

indicates data missing or illegible when filed

### Experimental Model and Subject Details

hiPSC Culture

The TkDA3 hiPSC clone was maintained in mTeSR1 medium on a growth factor-reduced (GFR) Matrigel (Matrigel: RPMI1640 medium=1:30)-coated culture dish. The 1383D2, 1383D6, M66, and YCU #7 hiPSC clones were maintained in StemFit AK02N medium on a laminin-511 (Laminin 511:PBS=1:150)-coated culture dish. All of the cells were maintained at 37° C. in a humidified incubator with a CO₂ concentration of 5%.

### PHH Culture

The cryopreserved PHHs were thawed according to the manufacturer's instructions. For the positive control experiments, PHHs were cultured on collagen I-coated culture dishes at a density of 1×10⁵ cells/cm² using Williams E medium containing 5% FBS, 1 μM dexamethasone, 100 IU/mL penicillin (Thermo Fisher Scientific), 100 μg/mL streptomycin (Thermo Fisher Scientific), 4 μg/mL Human Recombinant Insulin (Sigma), 2 mM GlutaMAX (Thermo Fisher Scientific), and 15 mM HEPES (Sigma). After 24 h, the supernatant from PHHs was collected for ALB analysis. For plasticity induction experiments, PHHs were seeded on Laminin-511-coated culture dishes at a density of 1×10⁴ cells/cm² using serum-free differentiation medium (SFD) (Nie et al., 2018a) containing 10 ng/mL FGF2, 10 ng/mL EGF, 20 ng/mL HGF, 10 μM Y-27632, 0.5 μM A83-01, 3 μM CHIR99021, and 0.25 mM NaB. At day 0 of culture, 5% FBS was used to improve cell adherence. After 24 h, the medium was exchanged with a fresh medium and subsequently changed every 2 days.

### Mice

Adult 7-9 week-old TK-NOG mice were used in this study (Hasegawa et al., 2011). To induce liver injury, TK-NOG recipients were injected intraperitoneally with ganciclovir (50 mg/kg, Mitsubishi Tanabe Pharma Corporation) twice, i.e., five and seven days prior to transplantation. The mice were housed in a temperature- and light-controlled (12 h light/dark cycle) specific pathogen-free animal facility, and were bred and maintained according to the Yokohama City University institutional guidelines for the use of laboratory animals. All experimental procedures were approved by the institutional review board of the Animal Research Center, Yokohama City University School of Medicine (No. 075).

### Method Details

Differentiation into hiPSC-Heps

Differentiation from hiPSC into hiPSC-Heps was performed using the following two steps:

Step I (Endoderm differentiation): hiPSC clones were differentiated into endoderm cells on a GFR Matrigel- or laminin 511-coated plate for 7 days using a medium consisting of RPMI 1640, 1% B27, 50 ng/mL WNT3A, and 100 ng/mL activin A. At day 0, 10 μM Y-27632 was added and from day 1 to day 3, 0.5 mM NaB was added. The medium was changed every day.
Step II (Differentiation into hepatocytes and their maturation): Using 0.05% trypsin/EDTA (Gibco), hiPSC endoderm cells were detached from the plate and reseeded into a GFR Matrigel- or laminin 511-coated plate using a nicotinamide-free hepatic differentiation medium (HDM) at a density of 1.5˜2×10⁵ cell/cm². From day 8 to 10, the medium was exchanged with HDM every day; from day 11, the medium was changed every 2 days. The HDM consisted of SFD containing 10 ng/mL FGF2, 20 ng/mL HGF, 10 ng/mL OSM, 100 nM dexamethasone and 10 mM nicotinamide. The control hiPSC-Heps were differentiated according to published protocols with minor modifications added (Kajiwara et al., 2012; Si-Tayeb et al., 2010).
Induction of Proliferative Hepatocytes (pHC)

Using 0.05% Trypsin/EDTA, the newly generated hiPSC-Heps were collected at certain aging times and reseeded on a GFR Matrigel- or Laminin 511-coated plate at a density of 5,000 cells/cm² using a reprogramming medium (RM) consisting of SFD containing 10 ng/mL FGF2, 10 ng/mL EGF, 20 ng/mL HGF, 10 μM Y-27632, 0.5 μM A83-01, and 3 μM CHIR99021. The medium was changed on day 1, 3, and 5. To block the PI3K and MAPK signaling pathways, 10 μM LY29002 and a given dose of PD0325902 (0.01 μM˜1 μM) were each added to RM. To block the function of EZH2, 0.1 μM DZNep was added in RM. To regulate epigenetic modification, 0.25 mM NaB, 0.5 mM VPA, 10 μM Trany, 0.5 μM RG108 and 0.5 μM BIX were added in RM. The number of cells was analyzed using an Incell analyzer 2000 (GE Healthcare) combined with Hoechst 33342 staining (Invitrogen).

The Subculture of Proliferative Hepatic Cells (pHC)

On day 6˜8 of induction, proliferative hepatic cells (pHC) were harvested using 0.05% Trypsin/EDTA and seeded on a GFR-Matrigel-coated or laminin-511-coated plate at a density of 1×10⁴ cells/cm² using RM. The medium was changed with Y-27632-free RM on days 1 and 3.

Time-Lapse Tracing of Hepatocyte Proliferation Induced from a Single Cell

D22-Hep was seeded on a GFR-Matrigel-coated plate (having RM) at a density of 500 cells/cm². After 4 h incubation, phase contrast imaging was performed using a BZ9000 all-in-one fluorescence microscope (Keyence), and images were taken every 24 h.

### Cell Counting Kit-8 Assay

Cell viability on the indicated days was determined using Cell Counting Kit-8 according to the manufacturer's instructions.

### Drawing of the Cell Growth Curve

pHCs were passaged at approximately 90% confluence, and the total cell number was determined at each passage. Growth rate=(total cell number)/(seeded cell number). The growth rates of pHCs were measured to calculate the cell growth curve.

### Calculation of the Population Doubling Times

pHCs were seeded on GFR Matrigel-coated plates at a density of 5000 cells/cm². After 4 h incubation, the medium was changed with a fresh medium, and the number of adherent cells was counted using an Incell analyzer 2000 in combination with Hoechst 33342 staining. The cell number was also counted at the following time points: 24 h, 48 h, and 72 h. The population doubling times were calculated using GraphPad Prism according to the cell number at each time point.

Hepatocyte Differentiation from pHC

The hepatocyte differentiation of pHCs was initiated at about 90% confluence. For 8 days, the medium was changed to HDM containing 3 μM RA and for another 7 days, the medium was changed to RA-free HDM. The medium was changed every 2 days. The total hepatocyte differentiation period was 15 days.

Cholangiocyte Differentiation from pHC

pHCs either during subculture or obtained by inducing plasticity at certain time points in the aging process were suspended in RM at a density of 1×10⁶ cells/mL. A total of 5,000 pHCs mixed with 50 μL GFR-Matrigel were seeded on a non-treated 24-well plate as a mount drop and incubated at 37° C. for 30 min in a 5% CO₂ incubator. Then, 700 μL of a cholangiocyte differentiation medium (CDM) was filled into the wells. The CDM consisted of SFD containing 10 ng/mL EGF, 20 ng/mL HGF, 50 ng/mL WNT3A, 100 ng/mL R-spondin-1, 50 ng/mL FGF10, 3 μM RA, 10 μM Y-27632 and 10 μM forskolin. The medium was changed every 3 days, and the related analyses were performed on day 10.

### Assays of Human ALB, A1AT, Ferritin, and AFP

Human ALB, A1AT, ferritin, and AFP were measured using a human albumin ELISA quantitation set, a human alpha-1-antitrypsin ELISA quantitation kit, a human ferritin ELISA quantitation kit, and a human AFP ELISA quantitation kit according to the respective manufacturers' instructions. Samples were diluted over a range from 10- to 5000-fold to obtain values falling within the linear range of the standard curve.

### Detection of Intracellular Senescence-Associated Beta-Galactosidase and ROS

The activity of senescence-associated beta-galactosidase (SA-β-Gal) was detected using a cellular senescence detection and quantification kit, and the intracellular ROS was analyzed using the CellROX™ Deep Red Reagent according to the manufacturer's instructions. Photographs were taken using a Leica TCS SP5 confocal microscope (Leica).

### Acetyl-CoA Assay

To determine acetyl-CoA levels in hiPSC-Heps, cells were detached from the plate using Cell Lysis Buffer. The concentration of acetyl-CoA was measured using an acetyl-coenzyme A assay kit according to the manufacturer's instructions. The obtained values were normalized using the corresponding protein concentrations.

### Indocyanine Green Uptake and Release

Indocyanine green (ICG) dry powder (DAIICHI SANKYO) (10 mg) was dissolved in 10 mL of a hepatocyte culture medium to obtain a 1 mg/mL stock. The cells, either in suspension or being seeded, were incubated with ICG for 4 h at 37° C. in a humidified incubator having a CO₂ concentration of 5%. Then, cells were washed three times with phosphate-buffered saline (PBS) and incubated in fresh HCM (hepatocyte culture medium BulletKit™ HCM™) for another 2 h to determine the ICG release. The images were captured using a BZ9000 all-in-one fluorescence microscope.

### Periodic Acid-Schiff Staining and Low-Density Lipoprotein Uptake

Periodic acid-Schiff (PAS) staining was used to detect glycogen according to the manufacturer's instructions. Briefly, cells were washed with PBS and fixed using 4% paraformaldehyde for 15 min at room temperature. After washing with PBS, the cells were oxidized in 0.5% periodic acid solution for 7 min, then washed with PBS and incubated in Schiff reagent for 15 min. After three rounds of incubation for 2 min each in aqueous sulfurous acid, the cells were washed with PBS and visualized by a BZ9000 all-in-one fluorescence microscope.

For low-density lipoprotein uptake analysis, cells were incubated with 5 μg/mL Dil-Ac-LDL and 50 μL of Hoechst 33342 standard diluted solution for 2 h at 37° C. Then, the cells were washed with PBS. Photographs were analyzed using a BZ9000 all-in-one fluorescence microscope.

### Transport Assay for Rhodamine 123 and Cholyl-Lysyl-Fluorescein

The rhodamine 123 transport assay and cholyl-lysyl-fluorescein (CLF) transport assay were performed according to previously described methods (Sampaziotis et al., 2015). For the rhodamine 123 transport assay, cholangiocytes were incubated with or without 10 μM verapamil at 37° C. for 30 min. Next, the cells were incubated with 100 μM rhodamine 123 for 5 min at 37° C. and then washed three times with IMDM. Fresh CDM was added and incubated at 37° C. for another 40 min. For the CLF transport assay, cholangiocytes were loaded with 5 μM CLF or 5 μM FITC for 30 min at 37° C. and then the cells were washed with IMDM three times. Fresh CDM was added and incubated at 37° C. for another 10 min. Photos were taken using a Leica TCS SP5 confocal microscope.

### Transmission Electron Microscopy

Transmission electron microscopic analysis of cholangiocyte cysts was performed according to a previously described method (Nie et al., 2018a). Briefly, pre-fixed cholangiocyte cysts were post-fixed, dehydrated, and embedded in a fresh 100% resin. Next, 70 nm ultra-thin sections were cut out and stained with 2% uranyl acetate. Then, the sections were washed with distilled water and stained with a lead stain solution. Grids were observed under a JEM-1400Plus microscope (JEOL), and digital images were captured using a VELETA camera (Olympus).

### Flow Cytometry

Antibodies used for flow cytometry were as follows: PE-mouse anti-human CXCR4, BV421 mouse anti-human CD117, APC mouse anti-human EpCAM, rabbit anti-H3K9ac, rabbit anti-H3K14ac, rabbit anti-H3K18ac, rabbit anti-H3K27ac, rabbit anti-H3K56ac, and goat anti-rabbit IgG (H+L) Alexa Fluor 647. Cells were acquired on a MoFlo Astrios system (Beckman Coulter).

Chromatin Immunoprecipitation (ChIP) Coupled with Quantitative PCR

The procedure performed was according to the manufacturer's instructions. Briefly, 3×10⁶ cells were cross-linked at room temperature in 1% formaldehyde for 10 min and in 1× Glycine for 5 min, harvested by scraping, centrifuged, and resuspended in lysis buffer. The DNA was digested into approximately 150˜900 bp long fragments by micrococcal nuclease. Using a Bioruptor ultrasonicator (Cosmo bio), the nuclear membrane was broken to release DNA fragments. The samples were immunoprecipitated overnight at 4° C. using an antibody cocktail containing rabbit anti-human H3K9ac, rabbit anti-human H3K18ac, and rabbit anti-human H3K27ac. Immunocomplexes were captured using 30 μL of protein G magnetic beads according to the manufacturer's instructions. The DNA was recovered by 2 h digestion at 65° C. using proteinase K. The DNA was purified using spin columns. Subsequently, ChIP and inputs were used for qPCR using the primers described in the following table.

### List of ChIP-PCR Primers and Probes for EZH2 Enhancers

TABLE 4

| Name | Left primer | Right primer | Probe |
|---|---|---|---|
| Pr1 | AGATCCT | GGAGAAA | #56 |
| (GH07J148882) | GTGTGAT | TGTTTGT | |
| | AAAGGAA | TGCCTAA | |
| | GTT | GA | |
| Pr2 | GCCGGAC | CTGGACA | #2 |
| (GH07J148882) | CCGTTAC | ACCAGAG | |
| | TACTTT | CGAAA | |
| Pr3 | ACTTTCA | TCTTCTC | #3 |
| (GH07J148940) | AGGAAGC | AGGCATT | |
| | CTATTGG | TTGAAGG | |
| | TTA | | |
| Pr4 | AGGTGTT | TAAGCAA | #42 |
| (GH07J148940) | CATCGGT | CTGCTGG | |
| | TTCACC | CTGACA | |

Sequences of Left and Right primers of Pr1: SEQ ID Nos: 1 and 2.
Sequences of Left and Right primers of Pr2: SEQ ID Nos: 3 and 4.
Sequences of Left and Right primers of Pr3: SEQ ID Nos: 5 and 6.
Sequences of Left and Right primers of Pr4: SEQ ID Nos: 7 and 8.
Manufacturer names and product numbers of probes are listed below.

| Probe | manufacturer | product number | |
|---|---|---|---|
| #56 | Roche | 04688538001 | Universal ProbeLibrary Probe 056(2 × 125 ul |
| #2 | Roche | 04684982001 | Universal ProbeLibrary Probe 002(2 × 125 ul |
| #3 | Roche | 04685008001 | Universal ProbeLibrary Probe 003(2 × 125 ul |
| #42 | Roche | 04688015001 | Universal ProbeLibrary Probe 042(2 × 125 ul |

### Transplantation

Following two ganciclovir injections, serum alanine aminotransferase (ALT) and aspartate aminotransferase (AST) were detected using DRI-CHEM (Fujifilm) according to the manufacturer's instructions. A total of 1×10⁶ PHHs or PHH-pHCs were intrasplenically transplanted into TK-NOG recipients. The serum was collected every 2 weeks, and mice were sacrificed at 12 weeks.

### RNA Isolation and Quantitative Real-Time Polymerase Chain Reaction (Q-PCR)

Total RNA was isolated using a PureLink™ RNA Mini Kit. RNA (<2 μg) was used as a template for single-strand cDNA synthesis using a high-capacity cDNA reverse transcription kit according to the manufacturer's instructions. Q-PCR was performed using cDNA as well as specific primers and universal probe library probes. The primers and probes used in this study are listed in the following table. All data were calculated by the ΔΔCT method using β-ACTB (Thermo Fisher Scientific) as a normalization control.

### List of Quantitative Q-PCR Primers and Probes for Human Genes

TABLE 5

| Gene | Left primer | Right primer | Probe |
|---|---|---|---|
| OCT4 | CTTCGCA | GAGAAGG | #60 |
| | AGCCCTC | CGAAATC | |
| | ATTTC | CGAAG | |
| NANOG | ATGCCTC | CAGGGCT | #69 |
| | ACACGGA | GTCCTGA | |
| | GACTGT | ATAAGC | |
| SOX17 | ACGCCGA | TCTGCCT | #61 |
| | GTTGAGC | CCTCCAC | |
| | AAGA | GAAG | |
| FOXA2 | AGACGGT | CGGGTGA | #7 |
| | GTTGCAG | AGAAGAG | |
| | AGACG | TGCTG | |
| TBX3 | AAAAATA | ACTGCAC | #47 |
| | GACAAGA | GGTGAGC | |
| | ACCCTTT | TGTTTT | |
| | TGC | | |
| TTR | GCCGTGG | CAGCTCT | #66 |
| | ATGTGTT | CCAGACT | |
| | CAGA | CACTGGT | |
| A1AT | ACCTGGG | GATCCTC | #82 |
| | ACAGTGA | AGCCAGG | |
| | ATCGAC | GAGAC | |
| AL6 | AATGTTG | CTTCCCT | #27 |
| | CCAAGCT | TCATCCC | |
| | GCTGA | GAAGTT | |
| TDO2 | AAGAGGA | AGCACCT | #83 |
| | ATTCATA | CTTTTTG | |
| | AGGATTC | CTTCTGA | |
| | AGG | | |
| G6PC | CCCTGTA | AAACAGT | #62 |
| | ACCTGTG | AGATGTG | |
| | AGACTGG | ACCATCA | |
| | | CCTA | |
| ASGR1 | GCTGGAG | CGCAGGT | #19 |
| | AAACAGC | GAGACAG | |
| | AGAAGG | GAACT | |
| HNF4A | AGCAACG | TCAGACC | #27 |
| | GACAGAT | CTGAGCC | |
| | GTGTGA | ACCT | |
| TAT | TGAAGTT | TCCCGAC | #37 |
| | ACGCAGG | TGGATAG | |
| | CAATGA | GAAGC | |
| CYP2C9 | TGGATGA | AGGAATA | #7 |
| | AGGTGGG | AAAACAG | |
| | AATTTT | CTGCATG | |
| | | C | |
| CYP2C19 | ACTATTG | TCTCAGG | #33 |
| | AAAACTT | GTTCTGC | |
| | GGTAATC | TTGTTG | |
| | ACTGC | | |
| CYP3A4 | GATGGCT | AGTCCAT | #2 |
| | CTCATCC | GTGAATG | |
| | CAGACTT | GGTTCC | |
| CYP7A1 | GCTATTC | TTGGCAC | #26 |
| | TTCGAAT | CAAATTG | |
| | TAGGAGA | CAGAG | |
| | AGG | | |
| MK167 | AGAGGTG | TGTCCCT | #30 |
| | TGGAGAA | ATGACTT | |
| | AATCCAA | CTGGTTC | |
| | | TTA | |
| PCNA | TGGAGAA | GAACTGG | #69 |
| | GTTGGAA | TTCATTC | |
| | RTGGAAA | ATCTCTA | |
| | C | TGG | |
| CCNB1 | TTTCGCC | GCACATC | #34 |
| | TGAGCCT | CAGATGT | |
| | ATTTTG | TTCCATT | |
| CCND1 | GACCTTC | GGTTCAG | #87 |
| | GTTGCCC | GCCTTGG | |
| | TCTGT | ACTG | |
| CCNE1 | GGCCAAA | GGGTCTG | #36 |
| | ATCGACA | CACAGAC | |
| | GGAC | TGCAT | |
| CDC20 | CTGTCTG | TCCTTGT | #58 |
| | AGTGCCG | AATGGGG | |
| | TGGAT | AGACCA | |
| EZH2 | TGTGGAT | GACGAGC | #36 |
| | ACTCCTC | CGTCCTT | |
| | CAAGGAA | TTTCA | |
| EpCAM | CCATETG | TGTGTTT | #3 |
| | CTGGTCT | TAGTTCA | |
| | GTGAA | ATGATGA | |
| | | TCCA | |
| C-MET | AAATGTG | TCTCTGA | #59 |
| | CATGAAG | ATTAGAG | |
| | CAGGAAG | CGATGTT | |
| | | GA | |
| LGR5 | ACCAGAC | TTCCCAG | #78 |
| | TATGCCT | GGAGTGG | |
| | TTGGAAA | ATTCTAT | |
| | C | | |
| RBP4 | TTTCTTT | CCAGAAG | #17 |
| | CTGATCT | CGCAGAA | |
| | GCCATCG | GATTG | |
| SOX9 | GTACCCG | TCTCGCT | #61 |
| | CACTTGC | CTCGTTC | |
| | ACAAC | AGAAGTC | |
| HNF6 | CCTGGAG | TTCTTTC | #88 |
| | CAAACTC | CTTTTGC | |
| | AAATCC | ATGCTG | |
| GGT | TGGATGT | CAGATCG | #44 |
| | GACTGAG | GGTGAGT | |
| | CTGGTC | GGTGTC | |
| CFTR | GAAGTAG | GCTTTCT | #52 |
| | TGATGGA | CAAATAA | |
| | GAATGTA | TTCCCCA | |
| | ACAGC | AA | |
| AQP1 | ATCCTCT | GCCCGAG | #48 |
| | CAGGCAT | TTCACAC | |
| | CACCTC | CATC | |
| SSTR2 | GGACCTA | TCACCTT | #47 |
| | GCGGATT | AATGGAC | |
| | GCAG | CCTGGA | |
| ACLY | TTCGTCC | ACATCAC | #31 |
| | CCCACAG | CCACGTC | |
| | TCAG | CACAG | |
| POH8 | GAGGCTG | GAAATTG | #17 |
| | GCCACAG | AACGCAG | |
| | TTTG | GACGTT | |
| ACSS2 | CCCCAAT | CACTCGG | #34 |
| | TAAGAGC | GCTCACA | |
| | TCATGC | CTCAT | |
| CPT1A | CAGTGGG | TCTCATG | #55 |
| | AGCGGAT | TGCTGGA | |
| | GTTTA | TGGTGT | |

Sequences of Left and Right primers for OCT4: SEQ ID Nos: 9 and 10.
Sequences of Left and Right primers for NANOG: SEQ ID Nos: 11 and 12.
Sequences of Left and Right primers for SOX17: SEQ ID Nos: 13 and 14.
Sequences of Left and Right primers for FOXA2: SEQ ID Nos: 15 and 16.
Sequences of Left and Right primers for TBX3: SEQ ID Nos: 17 and 18.
Sequences of Left and Right primers for TTR: SEQ ID Nos: 19 and 20.
Sequences of Left and Right primers for A1AT: SEQ ID Nos: 21 and 22.
Sequences of Left and Right primers for ALB: SEQ ID Nos: 23 and 24.
Sequences of Left and Right primers for TDO2: SEQ ID Nos: 25 and 26.
Sequences of Left and Right primers for G6PC: SEQ ID Nos: 27 and 28.
Sequences of Left and Right primers for ASGR1: SEQ ID Nos: 29 and 30.
Sequences of Left and Right primers for HNF4A: SEQ ID Nos: 31 and 32.
Sequences of Left and Right primers for TAT: SEQ ID Nos: 33 and 34.
Sequences of Left and Right primers for CYP2C9: SEQ ID Nos: 35 and 36.
Sequences of Left and Right primers for CYP2C19: SEQ ID Nos: 37 and 38.
Sequences of Left and Right primers for CYP3A4: SEQ ID Nos: 39 and 40.
Sequences of Left and Right primers for CYP7A1: SEQ ID Nos: 41 and 42.
Sequences of Left and Right primers for MKI67: SEQ ID Nos: 43 and 44.
Sequences of Left and Right primers for PCNA: SEQ ID Nos: 45 and 46.
Sequences of Left and Right primers for CCNB1: SEQ ID Nos: 47 and 48.
Sequences of Left and Right primers for CCND1: SEQ ID Nos: 49 and 50.
Sequences of Left and Right primers for CCNE1: SEQ ID Nos: 51 and 52.
Sequences of Left and Right primers for CDC20: SEQ ID Nos: 53 and 54.
Sequences of Left and Right primers for EZH2: SEQ ID Nos: 55 and 56.
Sequences of Left and Right primers for EpCAM: SEQ ID Nos: 57 and 58.
Sequences of Left and Right primers for C-MET: SEQ ID Nos: 59 and 60.
Sequences of Left and Right primers for LGR5: SEQ ID Nos: 61 and 62.
Sequences of Left and Right primers for RBP4: SEQ ID Nos: 63 and 64.
Sequences of Left and Right primers for SOX9: SEQ ID Nos: 65 and 66.
Sequences of Left and Right primers for HNF6: SEQ ID Nos: 67 and 68.
Sequences of Left and Right primers for GGT: SEQ ID Nos: 69 and 70.
Sequences of Left and Right primers for CFTR: SEQ ID Nos: 71 and 72.
Sequences of Left and Right primers for AQP1: SEQ ID Nos: 73 and 74.
Sequences of Left and Right primers for SSTR2: SEQ ID Nos: 75 and 76.
Sequences of Left and Right primers for ACLY: SEQ ID Nos: 77 and 78.
Sequences of Left and Right primers for PDHB: SEQ ID Nos: 79 and 80.
Sequences of Left and Right primers for ACSS2: SEQ ID Nos: 81 and 82.
Sequences of Left and Right primers for CPT1A: SEQ ID Nos: 83 and 84.
Manufacturer names and product numbers of probes are listed below.

| Probe | Manufacturer | Product number | |
|---|---|---|---|
| #60 | Roche | 04688589001 | Universal ProbeLibrary Probe 060(2 × 125 ul |
| #69 | Roche | 04688686001 | Universal ProbeLibrary Probe 099(2 × 125 ul |
| #81 | Roche | 04689046001 | Universal ProbeLibrary Probe 081(2 × 125 ul |
| #7 | Roche | 04685059001 | Universal ProbeLibrary Probe 007(2 × 125 ul |
| #47 | Roche | 04688074001 | Universal ProbeLibrary Probe 047(2 × 125 ul |
| #66 | Roche | 04688651001 | Universal ProbeLibrary Probe 066(2 × 125 ul |
| #82 | Roche | 04689054001 | Universal ProbeLibrary Probe 082(2 × 125 ul |
| #27 | Roche | 04687582001 | Universal ProbeLibrary Probe 027(2 × 125 ul |
| #83 | Roche | 04689062001 | Universal ProbeLibrary Probe 083(2 × 125 ul |
| #62 | Roche | 04688619001 | Universal ProbeLibrary Probe 062(2 × 125 ul |
| #19 | Roche | 04686926001 | Universal ProbeLibrary Probe 019(2 × 125 ul |
| #27 | Roche | 04687582001 | Universal ProbeLibrary Probe 027(2 × 125 ul |
| #37 | Roche | 04687957001 | Universal ProbeLibrary Probe 037(2 × 125 ul |
| #7 | Roche | 04685059001 | Universal ProbeLibrary Probe 007(2 × 125 ul |
| #33 | Roche | 04687663001 | Universal ProbeLibrary Probe 033(2 × 125 ul |
| #2 | Roche | 04684982001 | Universal ProbeLibrary Probe 002(2 × 125 ul |
| #26 | Roche | 04687574001 | Universal ProbeLibrary Probe 026(2 × 125 ul |
| #30 | Roche | 04687639001 | Universal ProbeLibrary Probe 030(2 × 125 ul |
| #69 | Roche | 04688686001 | Universal ProbeLibrary Probe 069(2 × 125 ul |
| #34 | Roche | 04687671001 | Universal ProbeLibrary Probe 034(2 × 125 ul |
| #87 | Roche | 04689127001 | Universal ProbeLibrary Probe 087(2 × 125 ul |
| #36 | Roche | 04687949001 | Universal ProbeLibrary Probe 036(2 × 125 ul |
| #58 | Roche | 04688554001 | Universal ProbeLibrary Probe 058(2 × 125 ul |
| #35 | Roche | 04687680001 | Universal ProbeLibrary Probe 035(2 × 125 ul |
| #3 | Roche | 04685008001 | Universal ProbeLibrary Probe 003(2 × 125 ul |
| #59 | Roche | 04688562001 | Universal ProbeLibrary Probe 059(2 × 125 ul |
| #78 | Roche | 04689011001 | Universal ProbeLibrary Probe 078(2 × 125 ul |
| #17 | Roche | 04686900001 | Universal ProbeLibrary Probe 017(2 × 125 ul |
| #61 | Roche | 04688597001 | Universal ProbeLibrary Probe 061(2 × 125 ul |
| #88 | Roche | 04689135001 | Universal ProbeLibrary Probe 088(2 × 125 ul |
| #44 | Roche | 04688040001 | Universal ProbeLibrary Probe 044(2 × 125 ul |
| #52 | Roche | 04688490001 | Universal ProbeLibrary Probe 052(2 × 125 ul |
| #48 | Roche | 04688082001 | Universal ProbeLibrary Probe 048(2 × 125 ul |
| #47 | Roche | 04688074001 | Universal ProbeLibrary Probe 047(2 × 125 ul |
| #31 | Roche | 04687647001 | Universal ProbeLibrary Probe 031(2 × 125 ul |
| #17 | Roche | 04686900001 | Universal ProbeLibrary Probe 017(2 × 125 ul |
| #34 | Roche | 04687671001 | Universal ProbeLibrary Probe 034(2 × 125 ul |
| #55 | Roche | 04688520001 | Universal ProbeLibrary Probe 055(2 × 125 ul |

### Histological Test and Immunofluorescence Staining

Liver tissue samples were embedded in O.C.T. (optimal cutting temperature) compound (Sakura Finetek Japan), and 5-μm sections were prepared and mounted on MAS-GP type A-coated slides (Matsunami Glass). For immunofluorescence staining, sections or cultured cells were fixed in a 4% paraformaldehyde solution in PBS for 10 min, washed three times with PBS, and blocked for 30 min using 10% ECL prime blocking agent dissolved in PBS containing 0.3% Triton X-100, followed by another three washes with PBS. Then, sections or cells were incubated with primary antibodies in a blocking buffer at 4° C. overnight. The sections or cells were washed three times with PBS and incubated with a fluorescence-labeled secondary antibody for another 60 min at room temperature. Finally, the sections or cells were washed three times with PBS and covered with a mounting solution containing DAPI. Fluorescence was detected with a Zeiss Axio Imager M1 microscope (Carl Zeiss A G, Oberkochen, Germany). Antibodies used for immunofluorescence staining were as follows: rabbit anti-human AFP (1:100), goat anti-human ALB (1:100), mouse anti-human CK19 (1:50), rabbit anti-human A1AT (1:100), chicken anti-human A1AT (1:100), mouse anti-human KI67 (1:50), goat anti-human HNF4A (1:100), mouse anti-ZO1 (1:100), mouse anti-human E-cadherin (1:100), mouse anti-human SOX9 (1:100), F-ACTIN (1:100), mouse anti-human CK8/18 (1:100), mouse anti-human NuMA (1:100), donkey anti-mouse IgG (H+L) Alexa Fluor 488 (1:500), donkey anti-rabbit IgG (H+L) Alexa Fluor 488 (1:500), donkey anti-goat IgG (H+L) Alexa Fluor 555 (1:500), goat anti-guinea pig IgG (H+L) Alexa Fluor 555 (1:500) and goat anti-chicken IgY (H+L) Alexa Fluor 488 (1:500).

### Microarray and Data Analysis

Total RNA was prepared from hiPSC-derived cells (D22-Hep, D52-Hep, D72-Hep, and D22-pHC), PHHs (2M-PHH and 78Y-PHH), as well as PHH-pHCs (2M-pHC and 78Y-pHC) using a PureLink™ RNA Mini Kit. RNA for gene-expression profiling was hybridized using SurePrint G3 Human Gene Expression 8×60K (Agilent Technologies) according to the manufacturer's instructions. Data were normalized using GeneSpring. Gene ontology enrichment analysis and KEGG pathway analysis were performed using DAVID Bioinformatics Resources 6.8 (https://david.ncifcrf.gov/home.jsp) (Huang da et al., 2009). The STRING interaction networks were analyzed using STRING 9.05 (http://string905.embl.de/) (Franceschini et al., 2013).

### Statistics

Values were expressed as the mean±standard deviation (SD). The statistical significance of differences was evaluated using the Mann-Whitney U test when two groups were compared or using one-way ANOVA and Bonferroni's multiple comparison tests when multiple groups were compared; 0p<0.05 was considered statistically significant, Statistical analysis was performed using GraphPad Prism.

### Data and Software Availability

### Data Resources

The accession number for the microarray data reported in this paper is GEO: GSE131806.

### REFERENCES

Franceschini, A., Szklarczyk, D., Frankild, S., Kuhn, M., Simonovic, M., Roth, A., Lin, J., Minguez, P., Bork, P., von Mering, C., et al. (2013). STRING v9.1: protein-protein interaction networks, with increased coverage and integration. Nucleic acids research 41, D808-815.
Hasegawa, M., Kawai, K., Mitsui, T., Taniguchi, K., Monnai, M., Wakui, M., Ito, M., Suematsu, M., Peltz, G., Nakamura, M., et al. (2011). The reconstituted ‘humanized liver’ in TK-NOG mice is mature and functional. Biochemical and biophysical research communications 405, 405-410.
Huang da, W., Sherman, B. T., and Lempicki, R. A. (2009). Systematic and integrative analysis of large gene lists using DAVID bioinformatics resources. Nature protocols 4, 44-57. Kajiwara, M., Aoi, T., Okita, K., Takahashi, R., Inoue, H., Takayama, N., Endo, H., Eto, K., Toguchida, J., Uemoto, S., et al. (2012). Donor-dependent variations in hepatic differentiation from human-induced pluripotent stem cells. Proceedings of the National Academy of Sciences of the United States of America 109, 12538-12543.
Nie, Y. Z., Zheng, Y. W., Miyakawa, K., Murata, S., Zhang, R. R., Sekine, K., Ueno, Y., Takebe, T., Wakita, T., Ryo, A., et al. (2018a). Recapitulation of hepatitis B virus-host interactions in liver organoids from human induced pluripotent stem cells. EBioMedicine 35, 114-123.
Nie, Y. Z., Zheng, Y. W., Ogawa, M., Miyagi, E., and Taniguchi, H. (2018b). Human liver organoids generated with single donor-derived multiple cells rescue mice from acute liver failure. Stem cell research & therapy 9, 5.
Sampaziotis, F., de Brito, M. C., Madrigal, P., Bertero, A., Saeb-Parsy, K., Soares, F. A. C., Schrumpf, E., Melum, E., Karlsen, T. H., Bradley, J. A., et al. (2015). Cholangiocytes derived from human induced pluripotent stem cells for disease modeling and drug validation. Nature biotechnology 33, 845-852.
Si-Tayeb, K., Noto, F. K., Nagaoka, M., Li, J., Battle, M. A., Duris, C., North, P. E., Dalton, S., and Duncan, S. A. (2010). Highly efficient generation of human hepatocyte-like cells from induced pluripotent stem cells. Hepatology 51, 297-305.

All publications, patents and patent applications cited herein are incorporated herein by reference in their entirety.

### INDUSTRIAL APPLICABILITY

The present invention can be used for mass production of hepatocytes and cholangiocytes. The human hepatocytes, etc. created by the present invention can be applied to in vitro drug metabolism tests, hepatotoxicity tests, hepatitis virus infection tests, or regenerative medicine, bioartificial liver, or the like. The human hepatocytes, etc. created by the present invention can be used to produce a chimeric animal the hepatocytes of which have been replaced with human hepatocytes; by using this chimeric animal, it is possible to conduct drug metabolism tests or safety tests on seed compounds for drug discovery. In addition, the present invention can promote liver regeneration by the use of the drug causing histone hyperacetylation.

1: A method for producing hepatocytes, comprising differentiating endoderm cells into hepatocytes in the presence of a member of FGF2, HGF, a member of the IL6 family, and dexamethasone, wherein the endoderm cells are cells differentiated from pluripotent stem cells by culturing the pluripotent stem cells in a medium containing B27, WNT3A and activin A.

2. (canceled)

3: The method according to claim 1, wherein the member of the IL6 family is at least one selected from the group consisting of oncostatin, IL-6, IL-11, IL-27, IL-35, IL-39, LIF, CT-1, CNTF, and CLCF1.

4. (canceled)

5: The method according to claim 1, wherein the endoderm cells are derived from human.

6: Hepatocytes produced by the method according to claim 1, wherein the hepatocytes are capable of surviving for 15 days or more while maintaining an albumin secretion ability in ex vivo monolayer culture.

7: The hepatocytes according to claim 6, wherein the hepatocytes are capable of surviving for 15 days or more while maintaining functionality as hepatocytes in addition to the albumin secretion ability.

8: The hepatocytes according to claim 7, wherein the functionality as hepatocytes, in addition to the albumin secretion ability, is at least one selected from the group consisting of drug metabolism, uptake and release of indocyanine green, glycogen storage, uptake of a low-density lipoprotein, and gene expression.

9: The hepatocytes according to claim 6, wherein the hepatocytes display aging-related characteristics.

10: The hepatocytes according to claim 9, wherein the aging-related characteristics is at least one selected from the group consisting of an increased cell volume, expression of aging-related genes, an increased inflammatory response, DNA damage, an increased level of an intracellular reactive oxygen species, an increased level of cellular senescence-associated β-galactosidase, an epigenetic change, a loss of telomere length, and a decline in mitochondrial function.

11: A method for producing A1AT negative hepatic progenitor cells, the method comprising culturing hepatocytes in the presence of a member of the FGF family.

12: The method according to claim 11, wherein the method comprises culturing hepatocytes in the presence of a member of the FGF family and a drug causing histone hyperacetylation.

13: The method according to claim 11, wherein the member of the FGF family is at least one selected from the group consisting of FGF2, FGF1, FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF22, and FGF23.

14: The method according to claim 12, wherein the drug causing histone hyperacetylation is a histone deacetylase inhibitor.

15: The method according to claim 12, wherein the histone deacetylase inhibitor is at least one selected from the group consisting of Trichostatin A, Tacedinaline (CI-994), M344, ITSA-1, Sodium valproate, Sodium 4-phenylbutuyrate, Sodium Butyrate (NaB), valproic acid (VPA), Abexinostat (PCI-24781), Belinostat (PXD101), Citarinostat (ACY-241), Dacinostat (LAQ824), Depudecin, Domatinostat (4SC-202), Droxinostat, Entinostat (MS-275, SNDX-275), Fimepinostat (CUDC-907), Givinostat (ITF2357), Mocetinostat (MGCD0103), Nexturastat A, Panobinostat (LBH-589,NVP-LBH589), Pracinostat (SB939), Quisinostat (JNJ-26481585) 2HCl, Resminostat, Ricolinostat (ACY-1215), Tucidinostat (Chidamide), Vorinostat (SAHA), ACY-738, Apicidin, AR-42, BG45, BML-210, BRD73954, CAY10603, CUDC-101, Curcumin, Depudecin, H1388, HC Toxin, HPOB, LMK-235, MC1568, Oxamflatin, (−)-Parthenolide, PCI-34051, RG2833 (RGFP109), RGFP966, Romidepsin (FK228, Depsipeptide), Santacruzamate A (CAY10683), Scriptaid, SKLB-23bb, Splitomicin, Suberoyl bis-hydroxamic acid, Tasquinimod, TH34, Tinostamustine (EDO-5101), TMP195, TMP269, Tubacin, and Tubastatin A.

16: The method according to claim 11, wherein the hepatocytes are cells differentiated from pluripotent stem cells, cells obtained by passaging cells differentiated from pluripotent stem cells, primary culture cells isolated from a biological tissue, cells obtained by passaging primary culture cells isolated from a biological tissue, or a combination thereof.

17: The method according to claim 11, wherein the hepatocytes are derived from human.

18: Alpha-fetoprotein (AFP)-negative hepatic progenitor cells produced using the method according to claim 11, the AFP-negative hepatic progenitor cells having a proliferative ability and a bidirectional differentiation ability into hepatocytes and bile duct epithelial cells.

19: The hepatic progenitor cells according to claim 18, wherein the hepatic progenitor cells are capable of differentiating into hepatocytes and/or cholangiocytes.

20: A method for producing hepatocytes, comprising inducing differentiation of the hepatic progenitor cells according to claim 18 into hepatocytes.

21: A method for producing cholangiocytes, comprising inducing differentiation of the hepatic progenitor cells according to claim 18 into cholangiocytes.

22-42. (canceled)


SEQUENCE LISTING (84 sequences)

SEQ ID NO: 1 - DNA, 24 bp, Artificial Sequence
  primer
agatcctgtgtgataaaggaagtt

SEQ ID NO: 2 - DNA, 23 bp, Artificial Sequence
  primer
ggagaaatgtttgttgcctaaga

SEQ ID NO: 3 - DNA, 20 bp, Artificial Sequence
  primer
gccggacccgttactacttt

SEQ ID NO: 4 - DNA, 19 bp, Artificial Sequence
  primer
ctggacaaccagagcgaaa

SEQ ID NO: 5 - DNA, 24 bp, Artificial Sequence
  primer
actttcaaggaagcctattggtta

SEQ ID NO: 6 - DNA, 21 bp, Artificial Sequence
  primer
tcttctcaggcattttgaagg

SEQ ID NO: 7 - DNA, 20 bp, Artificial Sequence
  primer
aggtgttcatcggtttcacc

SEQ ID NO: 8 - DNA, 20 bp, Artificial Sequence
  primer
taagcaactgctggctgaca

SEQ ID NO: 9 - DNA, 19 bp, Artificial Sequence
  primer
cttcgcaagccctcatttc

SEQ ID NO: 10 - DNA, 19 bp, Artificial Sequence
  primer
gagaaggcgaaatccgaag

SEQ ID NO: 11 - DNA, 20 bp, Artificial Sequence
  primer
atgcctcacacggagactgt

SEQ ID NO: 12 - DNA, 20 bp, Artificial Sequence
  primer
cagggctgtcctgaataagc

SEQ ID NO: 13 - DNA, 18 bp, Artificial Sequence
  primer
acgccgagttgagcaaga

SEQ ID NO: 14 - DNA, 18 bp, Artificial Sequence
  primer
tctgcctcctccacgaag

SEQ ID NO: 15 - DNA, 19 bp, Artificial Sequence
  primer
agacggtgttgcagagacg

SEQ ID NO: 16 - DNA, 19 bp, Artificial Sequence
  primer
cgggtgaagaagactgctg

SEQ ID NO: 17 - DNA, 24 bp, Artificial Sequence
  primer
aaaaatagacaacaacccttttgc

SEQ ID NO: 18 - DNA, 20 bp, Artificial Sequence
  primer
actgcagggtgagctgtttt

SEQ ID NO: 19 - DNA, 18 bp, Artificial Sequence
  primer
gccgtgcatgtgttcaga

SEQ ID NO: 20 - DNA, 21 bp, Artificial Sequence
  primer
cagctctccagactcactggt

SEQ ID NO: 21 - DNA, 20 bp, Artificial Sequence
  primer
acctgggacagtgaatcgac

SEQ ID NO: 22 - DNA, 19 bp, Artificial Sequence
  primer
gatcctcagccagggagac

SEQ ID NO: 23 - DNA, 19 bp, Artificial Sequence
  primer
aatgttgccaagctgctga

SEQ ID NO: 24 - DNA, 20 bp, Artificial Sequence
  primer
cttcccttcatcccgaagtt

SEQ ID NO: 25 - DNA, 24 bp, Artificial Sequence
  primer
aagaggaattcataaggattcagg

SEQ ID NO: 26 - DNA, 21 bp, Artificial Sequence
  primer
agcacctctttttgcttctga

SEQ ID NO: 27 - DNA, 21 bp, Artificial Sequence
  primer
ccctgtaacctgtgagactgg

SEQ ID NO: 28 - DNA, 25 bp, Artificial Sequence
  primer
aaagagtagatgtgaccatcacgta

SEQ ID NO: 29 - DNA, 20 bp, Artificial Sequence
  primer
gctggagaaacagcagaagg

SEQ ID NO: 30 - DNA, 19 bp, Artificial Sequence
  primer
cgcaggtcagacacgaact

SEQ ID NO: 31 - DNA, 20 bp, Artificial Sequence
  primer
agcaacggacagatgtgtga

SEQ ID NO: 32 - DNA, 18 bp, Artificial Sequence
  primer
tcagaccctgagccacct

SEQ ID NO: 33 - DNA, 20 bp, Artificial Sequence
  primer
tgaagttacccaggcaatga

SEQ ID NO: 34 - DNA, 19 bp, Artificial Sequence
  primer
tcccgactggataggaagc

SEQ ID NO: 35 - DNA, 20 bp, Artificial Sequence
  primer
tggatgaaggtggcaatttt

SEQ ID NO: 36 - DNA, 22 bp, Artificial Sequence
  primer
aggaataaaaacagctccatgc

SEQ ID NO: 37 - DNA, 26 bp, Artificial Sequence
  primer
actattgaaaacttggtaatcactgc

SEQ ID NO: 38 - DNA, 20 bp, Artificial Sequence
  primer
tctcagggttgtgcttgttg

SEQ ID NO: 39 - DNA, 21 bp, Artificial Sequence
  primer
gatggctctcatcccagactt

SEQ ID NO: 40 - DNA, 20 bp, Artificial Sequence
  primer
agtccatgtgaatgggttcc

SEQ ID NO: 41 - DNA, 25 bp, Artificial Sequence
  primer
gcttattcttggaattaggagaagg

SEQ ID NO: 42 - DNA, 19 bp, Artificial Sequence
  primer
ttggcaccaaattgcagag

SEQ ID NO: 43 - DNA, 21 bp, Artificial Sequence
  primer
agaggtgtgcagaaaatccaa

SEQ ID NO: 44 - DNA, 24 bp, Artificial Sequence
  primer
tgtccctatgacttctggttctta

SEQ ID NO: 45 - DNA, 22 bp, Artificial Sequence
  primer
tggagaacttggaaatggaaac

SEQ ID NO: 46 - DNA, 24 bp, Artificial Sequence
  primer
gaactggttcattcatctctatgg

SEQ ID NO: 47 - DNA, 20 bp, Artificial Sequence
  primer
tttcgcctgagcctattttg

SEQ ID NO: 48 - DNA, 21 bp, Artificial Sequence
  primer
gcacatccagatgtttccatt

SEQ ID NO: 49 - DNA, 19 bp, Artificial Sequence
  primer
gaccttcgttgccctctgt

SEQ ID NO: 50 - DNA, 18 bp, Artificial Sequence
  primer
ggttcaggccttgcactg

SEQ ID NO: 51 - DNA, 18 bp, Artificial Sequence
  primer
ggccaaaatcgacaggac

SEQ ID NO: 52 - DNA, 19 bp, Artificial Sequence
  primer
gggtctgcacagactgcat

SEQ ID NO: 53 - DNA, 19 bp, Artificial Sequence
  primer
ctgtctgagtgccgtggat

SEQ ID NO: 54 - DNA, 20 bp, Artificial Sequence
  primer
tccttgtaatggggagacca

SEQ ID NO: 55 - DNA, 21 bp, Artificial Sequence
  primer
tgtggatactcctccaaggaa

SEQ ID NO: 56 - DNA, 19 bp, Artificial Sequence
  primer
gaggagccgtcctttttca

SEQ ID NO: 57 - DNA, 19 bp, Artificial Sequence
  primer
ccatgtgctggtgtgtgaa

SEQ ID NO: 58 - DNA, 25 bp, Artificial Sequence
  primer
tgtgttttagttcaatgatgatcca

SEQ ID NO: 59 - DNA, 21 bp, Artificial Sequence
  primer
aaatgtgcatgaagcaggaag

SEQ ID NO: 60 - DNA, 23 bp, Artificial Sequence
  primer
tctctgaattagagcgatgttga

SEQ ID NO: 61 - DNA, 22 bp, Artificial Sequence
  primer
accagactatgcctttggaaac

SEQ ID NO: 62 - DNA, 21 bp, Artificial Sequence
  primer
ttcccagggagtggattctat

SEQ ID NO: 63 - DNA, 21 bp, Artificial Sequence
  primer
tttctttctgatctgccatcg

SEQ ID NO: 64 - DNA, 19 bp, Artificial Sequence
  primer
ccagaagcgcagaagattg

SEQ ID NO: 65 - DNA, 19 bp, Artificial Sequence
  primer
gtacccgcacttgcacaac

SEQ ID NO: 66 - DNA, 21 bp, Artificial Sequence
  primer
tctcgctctcgttcagaagtc

SEQ ID NO: 67 - DNA, 20 bp, Artificial Sequence
  primer
cctggagcaaactcaaatcc

SEQ ID NO: 68 - DNA, 20 bp, Artificial Sequence
  primer
ttctttccttttgcatgctg

SEQ ID NO: 69 - DNA, 20 bp, Artificial Sequence
  primer
tggatgtgactgaggtggtc

SEQ ID NO: 70 - DNA, 20 bp, Artificial Sequence
  primer
gagatcgggtgagtggtgtc

SEQ ID NO: 71 - DNA, 26 bp, Artificial Sequence
  primer
gaagtagtgatggagaatgtaacagc

SEQ ID NO: 72 - DNA, 23 bp, Artificial Sequence
  primer
gctttctcaaataattccccaaa

SEQ ID NO: 73 - DNA, 20 bp, Artificial Sequence
  primer
atcctctcaggcatcacctc

SEQ ID NO: 74 - DNA, 18 bp, Artificial Sequence
  primer
gcccgagttcacaccatc

SEQ ID NO: 75 - DNA, 18 bp, Artificial Sequence
  primer
ggagctagcggattgcag

SEQ ID NO: 76 - DNA, 20 bp, Artificial Sequence
  primer
tcaccttaatggaccctgga

SEQ ID NO: 77 - DNA, 18 bp, Artificial Sequence
  primer
ttcgtcccccacagtcag

SEQ ID NO: 78 - DNA, 19 bp, Artificial Sequence
  primer
acatcacccacgtccacac

SEQ ID NO: 79 - DNA, 18 bp, Artificial Sequence
  primer
gaggctggccacagtttg

SEQ ID NO: 80 - DNA, 20 bp, Artificial Sequence
  primer
gaaattgaacgcaggacctt

SEQ ID NO: 81 - DNA, 20 bp, Artificial Sequence
  primer
ccccaattaagaggtcatgc

SEQ ID NO: 82 - DNA, 19 bp, Artificial Sequence
  primer
cactcgggctcacactcat

SEQ ID NO: 83 - DNA, 19 bp, Artificial Sequence
  primer
cagtgggagcggatgttta

SEQ ID NO: 84 - DNA, 20 bp, Artificial Sequence
  primer
tctcatgtgctggatggtgt